(19) United States
(12) Patent Application Publication
(10) Pub. No.: US 2024/0209026 A1
(43) Pub. Date: Jun. 27, 2024

(54) A4B7 THIOETHER PEPTIDE DIMER ANTAGONISTS

(71) Applicant: Protagonist Therapeutics, Inc., Newark, CA (US)

(72) Inventors: Ashok BHANDARI, Pleasanton, CA (US); Dinesh V. PATEL, Fremont, CA (US); Genet ZEMEDE, San Jose, CA (US); Brian Troy FREDERICK, Ben Lomand, CA (US); Larry C. MATTHEAKIS, Cupertino, CA (US); David LIU, Milpitas, CA (US)

(73) Assignee: Protagonist Therapeutics, Inc., Newark, CA (US)

(21) Appl. No.: 18/501,864

(22) Filed: Nov. 3, 2023

Related U.S. Application Data
(63) Continuation of application No. 17/137,049, filed on Dec. 29, 2020, now Pat. No. 11,840,581.
Application No. 17/137,049 is a continuation of application No. 16/774,686, filed on Jan. 28, 2020, now abandoned.
Application No. 16/774,686 is a continuation of application No. 16/039,813, filed on Jul. 19, 2018, now Pat. No. 10,626,146.
Application No. 16/039,813 is a continuation of application No. 15/614,047, filed on Jun. 5, 2017, now Pat. No. 10,059,744.
Application No. 15/614,047 is a continuation of application No. 14/714,198, filed on May 15, 2015, now Pat. No. 9,714,270.
(60) Provisional application No. 62/058,499, filed on Oct. 1, 2014.
(60) Provisional application No. 62/058,501, filed on Oct. 1, 2014.
(60) Provisional application No. 61/994,717, filed on May 16, 2014.
(60) Provisional application No. 61/994,699, filed on May 16, 2014.

(51) Int. Cl. C07K 7/56; A61K 38/00; A61K 38/12; A61K 47/54; A61K 47/60; A61K 47/64; C07K 7/06; C07K 7/08; C07K 14/705

(52) U.S. Cl. CPC C07K 7/56; A61K 38/12; A61K 47/54; A61K 47/545; A61K 47/60; A61K 47/64; C07K 7/06; C07K 7/08; C07K 14/70546; A61K 38/00

(57) ABSTRACT

The invention relates to thioether monomer and dimer peptide molecules which inhibit binding of a4β7 to the mucosal addressing cell adhesion molecule (MAdCAM) in vivo.

### CROSS REFERENCE TO RELATED APPLICATIONS

This application is a Continuation of U.S. application Ser. No. 17/137,049, filed Dec. 29, 2020, which is a Continuation of U.S. application Ser. No. 16/774,686, filed Jan. 28, 2020, which is a Continuation of U.S. application Ser. No. 16/039,813, filed Jul. 19, 2018, now U.S. Pat. No. 10,626,146, issued Apr. 21, 2020; which is a Continuation of U.S. application Ser. No. 15/614,047, filed Jun. 5, 2017, now U.S. Pat. No. 10,059,744, issued Aug. 28, 2018; which is a Continuation of U.S. application Ser. No. 14/714,198, filed May 15, 2015, now U.S. Pat. No. 9,714,270, issued Jul. 25, 2017; which claims priority to U.S. Provisional Application No. 61/994,699, filed on May 16, 2014, U.S. Provisional Application No. 61/994,717, filed on May 16, 2014, U.S. Provisional Application No. 62/058,499, filed on Oct. 1, 2014, and U.S. Provisional Application No. 62/058,501, filed on Oct. 1, 2014, all of which are incorporated by reference herein in their entireties.

### SEQUENCE LISTING

The Sequence Listing XML associated with this application is provided in XML file format and is hereby incorporated by reference into the specification. The name of the XML file containing the Sequence Listing XML is PRTH_010_07US_ST26.xml. The XML file is 1,248,288 bytes, and created on Nov. 3, 2023, and is being submitted electronically via USPTO Patent Center.

### FIELD OF THE INVENTION

The present invention relates to the field of engineered peptides, and to the field of peptides that bind to integrins. In particular, the present invention relates to thioether peptides (e.g. thioether peptide monomers and dimers) that inhibit binding of a407 to the mucosal addressin cell adhesion molecule (MAdCAM) in vitro, and show high selectivity against α4β1 binding.

### BACKGROUND OF THE INVENTION

Integrins are noncovalently associated α/β heterodimeric cell surface receptors involved in numerous cellular processes ranging from cell adhesion and migration to gene regulation (Dubree, et al., Selective α4β7 Integrin Antagonist and Their Potential as Anti-inflammatory Agents, J. Med. Chem. 2002, 45, 3451-3457). Differential expression of integrins can regulate a cell's adhesive properties, allowing different leukocyte populations to be recruited to specific organs in response to different inflammatory signals. If left unchecked, the integrin-mediated adhesion process can lead to chronic inflammation and autoimmune disease.

The α4 integrins, α4β1 and α4β7, play essential roles in lymphocyte migration throughout the gastrointestinal tract. They are expressed on most leukocytes, including B and T lymphocytes, where they mediate cell adhesion via binding to their respective primary ligands, vascular cell adhesion molecule (VCAM), and mucosal addressin cell adhesion molecule (MAdCAM), respectively. The proteins differ in binding specificity in that VCAM binds both α4β1 and to a lesser extent α4β7, while MAdCAM is highly specific for α4β7. In addition to pairing with the α4 subunit, the β7 subunit also forms a heterodimeric complex with αE subunit to form αEβ7, which is primarily expressed on intraepithelial lymphocytes (IEL) in the intestine, lung and genitourinary tract. αEβ7 is also expressed on dendritic cells in the gut. The αEβ7 heterodimer binds to E-cadherin on the epithelial cells. The IEL cells are thought to provide a mechanism for immune surveillance within the epithelial compartment. Therefore, blocking αEβ7 and α4β7 together may be a useful method for treating inflammatory conditions of the intestine.

Inhibitors of specific integrins-ligand interactions have been shown effective as anti-inflammatory agents for the treatment of various autoimmune diseases. For example, monoclonal antibodies displaying high binding affinity for α4β7 have displayed therapeutic benefits for gastrointestinal auto-inflammatory/autoimmune diseases, such as Crohn's disease, and ulcerative colitis (Id). However, these therapies interfered with α4β1 integrin-ligand interactions thereby resulting in dangerous side effects to the patient. Therapies utilizing small molecule antagonists have shown similar side effects in animal models, thereby preventing further development of these techniques.

Accordingly, there is a need in the art for integrin antagonist molecules having high affinity for the α4β7 integrin and high selectivity against the α4β1 integrin, as a therapy for various gastrointestinal autoimmune diseases.

Such integrin antagonist molecules are disclosed herein.

### SUMMARY OF THE INVENTION

The present invention has been developed in response to the present state of the art, and in particular, in response to the problems and needs in the art that have not yet been fully solved by currently available integrin antagonists that are selective for α4β7. Thus, in certain aspects, the present invention provides α4β7 antagonist thioether peptide monomers and dimers for use as anti-inflammatory and/or immunosuppressive agents. Further, the present invention provides α4β7 antagonist thioether peptides (e.g. monomers and dimers for use in treating a condition that is associated with a biological function of α4β7 or on cells or tissues expressing MAdCAM.

Aspects of the invention relate to a novel class of cyclized, thioether peptidic compounds exhibiting integrin antagonist activity, namely, exhibiting high specificity for α4β7 integrin. In certain embodiments, each peptide of the present invention comprises a downstream natural or unnatural amino acid and an upstream modified amino acid or aromatic group that are capable of bridging to form a cyclized structure through a thioether bond. Peptides of the present invention demonstrate increased stability when administered orally as a therapeutic agent. The peptides of the present invention further provide increased specificity and potency as compared to analogs that are cyclized through a bond other than a thioether bond, e.g., a disulfide bond.

In certain embodiments, cyclized, thioether peptidic compounds exhibiting integrin antagonist activity are monomer peptides. In particular embodiments, the compounds of the present invention comprise dimerized peptides, each subunit of the dimer forming a cyclized structure through a thioether bond. The thioether cyclization feature provides the peptides of the present invention increased stability, specificity, and potency as compared to analogs that are cyclized through a bond other than a thioether bond, e.g., a disulfide bond. In some embodiments, dimerization of thioether peptide monomers further provides for increased specificity and potency as compared monomer analogs.

In one embodiment, the invention provides a peptide molecule comprising a structure of Formula (V):

(Formula (V)
(SEQ ID NO: 49)
Xaa¹-Xaa²-Xaa³-Xaa⁴-Xaa⁵-Xaa⁶-Xaa⁷-Xaa⁸-Xaa⁹-
Xaa¹⁰-Xaa¹¹-Xaa¹²-Xaa¹³-Xaa¹⁴

or a pharmaceutically acceptable salt thereof, wherein the peptide comprises a thioether bond between Xaa⁴ and Xaa¹⁰, and wherein:
Xaa¹ is absent, or Xaa¹ is any amino acid;
Xaa² is absent, or Xaa² is any amino acid;
Xaa³ is absent, or Xaa³ is any amino acid;
Xaa⁴ is an amino acid, aliphatic acid, alicyclic acid, or modified 2-methyl aromatic acid having a side chain with one or two carbons, and capable of forming a thioether bond with Xaa¹⁰;
Xaa⁵ is selected from the group consisting of N(alpha)-Me-Arg, Arg, HomoArg, Dap, Dab, Arg-Me-sym, Arg-Me-asym, 4-Guan, Cit, Cav, N-Me-Lys, Phe(4-quanidino), Phe(4-carbamoyl amino), Phe(4-NH₂), N-Me-HomoArg, Tyr, His, and suitable isostere replacements;
Xaa⁶ is selected from the group consisting of Ser, Gly, Thr, Ile, and suitable isostere replacements;
Xaa⁷ is selected from the group consisting of Asp, N-Me-Asp, Asp(OMe), D-Asp, and suitable isostere replacements;
Xaa⁸ is selected from the group consisting of Thr, Gln, Ser, Asp, Pro, Gly, His, Ala, Ile, Phe, Lys, Arg, Asn, Glu, Val, Tyr, Trp, Leu, Met, HomoLeu, Nle, and N-Methyl amino acids including N-Me-Thr;
Xaa⁹ is selected from the group consisting of Gln, Asn, Asp, Pro, Gly, Ala, Phe, Leu, Glu, Ile, Val, HLeu, n-Butyl Ala, n-Pentyl Ala, n-Hexyl Ala, Nle, cyclobutyl-Ala, Cpa, Aoc, N-Me-Leu, and suitable isostere replacements;
Xaa¹⁰ is selected from the group consisting of Cys, N-Me-Cys, D-Cys, HCys, Pen, D-Pen, and Pen(═O);
Xaa¹¹ is absent or is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Phe (2-carbomyl), Tyr(Me), HomoPhe, N-Me-Phe, N-Me-Tyr, Ser, Sar, Dihydro Trp, Ile, Leu, Ser, Arg, Thr, Sar, and Ser, aromatic amino acids, substituted aromatic amino acids, Gly, Gln, Asn, Asp, Ala, Ile, Leu, Val, Met, Thr, Lys, Trp, Tyr, His, Glu, Ser, Arg, Pro, Phe, Sar, 1-Nal, 2-Nal, D-1-Nal, D-2-Nal, HPhe, D-Phe, D-Tyr, Phe(4-F), O-Me-Tyr, dihydro-Trp, Dap, Dab, Dab(Ac), Orn, D-Orn, N-Me-Orn, N-Me-Dap, D-Dap, D-Dab, Bip, Ala(3,3diphenyl), Biphenyl-Ala, aromatic ring substituted Phe, aromatic ring substituted Trp, aromatic ring substituted His, hetero aromatic amino acids, N-Me-Lys, N-Me-Lys(Ac), 4-Me-Phe, Phe(4tBu), Phe(4-OMe), Phe(4-COOH), Phe(2-carbomyl), Phe(3-carbomyl), Phe(CF3), Phe(2,4-diCl), Phe(3,4-diCl), Aic, N-Me-Tyr, N-Me-Phe, Tic, Phe(4CF3), Bpa, Phe(3-Me), Phe(2-Me), Phe(2-CF3), P-Me-Phe, and corresponding D-amino acids and suitable isostere replacements;
Xaa¹² is absent or selected from the group consisting of aromatic amino acids, substituted aromatic amino acids, Glu, D-Glu, HomoGlu, Beta-Homo-Glu, Asp, D-HomoGlu, Amide, Lys, COOH, CONH₂, Gln, Pro, Gly, His, Ala, Ile, Phe, Arg, Leu, Val, Tyr, Trp, Met, Gla, Ser, Asn, D-Glu, β-HGlu, 2-Nal, 1-Nal, D-Asp, Bip, β-HPhe, β-Glu, D-Tyr, D-Phe, D-Lys, Dap, Dab, Orn, D-Om, N-Me-Orn, N-Me-Dap, N-Me-Dab, N-Me Lys, D-Dap, D-Dab, D-His, F(4-COOH), Tic, D-Trp, D-Leu, D-Arg, D-Thr, N-Me-Glu, N-Me-Asp, alpha-H-Glu, suitable isosteres, and corresponding D-amino acids;
Xaa¹³ is absent or any amino acid; and
Xaa¹⁴ is absent or any amino acid;
wherein if the peptide molecule is a peptide dimer or subunit thereof, then Xaa¹⁴ is absent or selected from the group consisting of: any amino acid with an amine side chain, Lys, D-Lys, N-Me-Lys, D-N-Me-Lys, Orn, N-Me-Orn, Dab, N-Me-Dab, Dap, N-Me-Dap, Homo-Lys, D-Dap, D-Dab, D-Orn, Gln, Pro, Gly, His, Ala, Ile, Phe, Lys, Arg, Leu, Val, Tyr, Trp, Met, Glu, Ser, Asn, Gla, Cys, HomoCys, COOH, CONH₂, suitable isosteres, corresponding D-amino acids, and corresponding N-Methyl amino acids, and wherein the peptide molecule comprises a thioether bond between Xaa⁴ and Xaa¹⁰.

In particular embodiments, Xaa¹, Xaa² and Xaa³ are absent. In certain embodiments, Xaa⁴ is a 2-methylbenzoyl moiety. In certain embodiments, Xaa⁵ is 2-Me-Arg. In particular embodiments, Xaa⁸ is selected from the group consisting of Thr, Gln, Ser, Asp, Gly, His, Ala, Ile, Phe, Lys, Arg, Asn, Glu, Val, Tyr, Trp, Leu, Met, HomoLeu, Nle, and N-Methyl amino acids including N-Me-Thr. In particular embodiments, Xaa⁹ is selected from the group consisting of Gln, Asn, Asp, Gly, Ala, Phe, Leu, Glu, Ile, Val, HLeu, n-Butyl Ala, n-Pentyl Ala, n-Hexyl Ala, Nle, cyclobutyl-Ala, Cpa, Aoc, N-Me-Leu, and suitable isostere replacements. In certain embodiments, Xaa¹⁴ is selected from the group consisting of: Lys, D-Lys, N-Me-Lys, D-N-Me-Lys, Orn, Dab, Dap, Homo-Lys, D-Dap, D-Dab, Cys, HomoCys, Pen, or D-Orn. In particular embodiments, Xaa¹⁴ is selected from the group consisting of: D-Lys, N-Me-Lys, and D-N-Me-Lys. In certain embodiments, the peptide molecule comprises N(alpha)methylation of at least one position selected from the group consisting of Xaa³, Xaa⁵, Xaa⁷-Xaa⁹, and Xaa¹¹-Xaa¹³. In certain embodiments, the peptide molecule comprises acylation for at least one position selected from the group consisting of Xaa¹-Xaa³ and Xaa¹¹-Xaa¹⁴.

In a related embodiment, the invention includes a peptide molecule comprising a structure of Formula (VI) (SEQ ID NO: 387):

(Formula VI)
Xaa¹-Xaa²-Xaa³-Xaa⁴-Xaa⁵-Xaa⁶-Xaa⁷-Xaa⁸-Xaa⁹-
Xaa¹⁰-Xaa¹¹

or a pharmaceutically acceptable salt thereof, wherein
Xaa¹ is a 2-Me-benzoyl group capable of forming a thioether bond with Xaa⁷;
Xaa² is selected from the group consisting of N(alpha)-Me-Arg, Arg, HArg, Dap, Dab, Arg-Me-sym, Arg-Me-asym, 4-Guan, Cit, Cav, and suitable isostere replacements;
Xaa³ is selected from the group consisting of Ser, Gly, and suitable isostere replacements;
Xaa⁴ is selected from the group consisting of Asp, N-Me-Asp, Asp(OMe), D-Asp, and a suitable isostere replacements;
Xaa⁵ is selected from the group consisting of Thr, Gln, Ser, Asp, Pro, Gly, His, Ala, Ile, Phe, Lys, Arg, Asn, Glu, Val, Tyr, Trp, Leu, Met, and N-Methyl amino acids including N-Me-Thr, and suitable isostere replacements;
Xaa⁶ is selected from the group consisting of Gln, Asn, Asp, Pro, Gly, Ala, Phe, Leu, Glu, Ile, Val, HLeu, n-Butyl Ala, n-Pentyl Ala, n-Hexyl Ala, Nle, cyclobutyl-Ala, N-Me-Leu, and suitable isostere replacements;
Xaa⁷ is selected from the group consisting of Cys, N-Me-Cys, D-Cys, HCys, Pen, and D-Pen;
Xaa⁸ is selected from the group consisting of absent, Gly, Gln, Asn, Asp, Ala, Ile, Leu, Val, Met, Thr, Lys, Trp, Tyr, His, Glu, Ser, Arg, Pro, Phe, Sar, 1-Nal, 2-Nal, HPhe, Phe(4-F), O-Me-Tyr, dihydro-Trp, Dap, Dab, Dab(Ac), Om, D-Orn, N-Me-Om, N-Me-Dap, D-Dap, D-Dab, Bip, Ala(3,3diphenyl), Biphenyl-Ala, aromatic ring substituted Phe, aromatic ring substituted Trp, aromatic ring substituted His, hetero aromatic amino acids, N-Me-Lys, N-Me-Lys(Ac), Bpa, Phe(3-Me), Phe(2-Me), Phe(2-CF3), P-Me-Phe, 4-Me-Phe, and corresponding D-amino acids and suitable isostere replacements;
Xaa⁹ is selected from the group consisting of absent, Glu, Amide, Lys, COOH, CONH₂, Gln, Pro, Gly, His, Ala, Ile, Phe, Lys, Arg, Leu, Val, Tyr, Trp, Met, Gla, Ser, Asn, D-Glu, β-HGlu, 2-Nal, 1-Nal, D-Asp, Bip, β-HPhe, β-Glu, D-Tyr, D-Lys, Dap, Dab, Orn, D-Orn, N-Me-Orn, N-Me-Dap, N-Me-Dab, N-Me Lys, D-Dap, D-Dab, Glu, N-Me-Asp, alpha-H-Glu, suitable isosteres, and corresponding D-amino acids;
Xaa¹⁰ is selected from the group consisting of absent, Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, N-Me-Lys, D-Dap, D-Dab, COOH, CONH₂, suitable isosteres, and corresponding D-amino acids; and
Xaa¹¹ is selected from the group consisting of absent, Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, N-Me-Lys, D-Dap, D-Dab, COOH, CONH₂, suitable isosteres, and corresponding D-amino acids, wherein the peptide further comprises a thioether bond between Xaa¹ and Xaa⁷,
wherein the peptide further comprises a thioether bond between Xaa¹ and Xaa⁷.

In particular embodiments, Xaa⁵ is selected from the group consisting of Thr, Gln, Ser, Asp, Gly, His, Ala, Ile, Phe, Lys, Arg, Asn, Glu, Val, Tyr, Trp, Leu, Met, and N-Methyl amino acids including N-Me-Thr, and suitable isostere replacements. In particular embodiments, Xaa⁶ is selected from the group consisting of Gln, Asn, Asp, Gly, Ala, Phe, Leu, Glu, Ile, Val, HLeu, n-Butyl Ala, n-Pentyl Ala, n-Hexyl Ala, Nle, cyclobutyl-Ala, N-Me-Leu, and suitable isostere replacements. In particular embodiments, any of the peptide molecules of the present invention, further comprise a terminal modifying group selected from the group consisting of DIG, PEG4, PEG13, PEG25, PEG1K, PEG2K, PEG4K, PEG5K, Polyethylene glycol having molecular weight from 400 Da to 40,000 Da, IDA, Ac-IDA, ADA, Glutaric acid, Isophthalic acid, 1,3-phenylenediacetic acid, 1,4-phenylenediacetic acid, 1,2-phenylenediacetic acid, AADA, suitable aliphatic acids, suitable aromatic acids, and heteroaromatic acids. In certain embodiments, the C-terminus of the peptide molecule further comprises a modifying group.

In certain embodiments, the peptide molecules are monomers.

In certain embodiments, the peptide molecules are dimers. In certain embodiments, a dimer comprises two peptide molecules of the present invention dimerized by a linker. In particular embodiments, the linker is selected from the group consisting of: DIG, PEG4, PEG4-biotin, PEG13, PEG25, PEG1K, PEG2K, PEG3.4K, PEG4K, PEG5K, IDA, ADA, Boc-IDA, Glutaric acid, Isophthalic acid, 1,3-phenylenediacetic acid, 1,4-phenylenediacetic acid, 1,2-phenylenediacetic acid, Triazine, Boc-Triazine, IDA-biotin, PEG4-Biotin, AADA, suitable aliphatics, aromatics, heteroaromatics, and polyethylene glycol based linkers having a molecular weight from approximately 400 Da to approximately 40,000 Da. In certain embodiments, the two peptide molecules are dimerized via their C-termini.

In another embodiment, the present invention includes a pharmaceutical composition comprising a peptide molecule of the invention and a pharmaceutically acceptable carrier, diluent or excipient. In particular embodiments, the pharmaceutical composition is formulated for oral delivery. In certain embodiments, it further comprises an enteric coating. In certain embodiments, the enteric coating releases the pharmaceutical composition within a subject's lower gastrointestinal system.

In a further related embodiment, the present invention provides a method for treating or preventing a disease or condition that is associated with a biological function of integrin α4β7, the method comprising providing to a subject in need thereof an effective amount of a peptide molecule of the invention or a pharmaceutical composition of the invention. In certain embodiments, the disease or condition is an inflammatory bowel disease. In particular embodiments, the inflammatory bowel disease is ulcerative colitis or Crohn's disease. In particular embodiments, the peptide molecule inhibits binding of α4β7 to MAdCAM. In certain embodiments, the peptide molecule or the pharmaceutical composition is provided to the subject in need thereof at an interval sufficient to ameliorate the condition. In certain embodiments, the interval is selected from the group consisting of around the clock, hourly, every four hours, once daily, twice daily, three times daily, four times daily, every other day, weekly, bi-weekly, and monthly. In particular embodiments, the peptide molecule or pharmaceutical composition is provided as an initial does followed by one or more subsequent doses, and the minimum interval between any two doses is a period of less than 1 day, and wherein each of the doses comprises an effective amount of the peptide molecule. In particular embodiments, the effective amount of the peptide molecule or the pharmaceutical composition is sufficient to achieve at least one of the following: a) about 50% or greater saturation of MAdCAM binding sites on α4β7 integrin molecules; b) about 50% or greater inhibition of α4β7 integrin expression on the cell surface; and c) about 50% or greater saturation of MAdCAM binding sites on α4β7 molecules and about 50% or greater inhibition of α4β7 integrin expression on the cell surface, wherein i) the saturation is maintained for a period consistent with a dosing frequency of no more than twice daily; ii) the inhibition is maintained for a period consistent with a dosing frequency of no more than twice daily; or iii) the saturation and the inhibition are each maintained for a period consistent with a dosing frequency of no more than twice daily. In certain embodiments, the peptide molecule is administered orally, parenterally, or topically.

### BRIEF DESCRIPTION OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a schematic showing C- and N-terminal dimerization via linker molecules according to certain representative embodiments of peptide dimers of the present invention. For example, in C-terminal dimerization, the NH₂ group may be a side chain of the C-terminal amino acid, and in N-terminal dimerization, the NH₂ group may be an N-terminal free amine group.

FIG. 2 is a schematic showing an integrin antagonist peptide dimer, comprising two thioether monomer subunits according to SEQ ID NO: 22, wherein the subunits are aligned and linked at their respective C-termini by a DIG linker moiety in accordance with a representative embodiment of the present invention. Lowercase k indicates D-Lysine.

FIG. 3 is a schematic showing a cyclized, thioether peptide monomer or monomer subunit of a dimer molecule according to SEQ ID NO: 1 (Formula (I)), wherein a thioether bond is formed between Xaa⁴ and Xaa¹⁰ in accordance with a representative embodiment of the present invention.

FIG. 4 is a schematic showing a cyclized, thioether peptide monomer or monomer subunit of a dimer molecule according to SEQ ID NO: 2 (Formula (II)), wherein Xaa¹ is a 2-methylbenzoyl moiety forming a thioether bond with Xaa⁷ in accordance with a representative embodiment of the present invention. Non-limiting examples of suitable chemical moieties for substitution at RT-R⁴ are provided and discussed below.

FIG. 5 is a diagram of an illustrative linker system that may be used to dimerize monomer subunits of dimer molecules of the present invention, e.g., dimerization through a sulfhydryl group. FIG. 5 shows a pair of integrin antagonist monomer subunits wherein the subunits are aligned and linked at their respective C-termini by a linker that connects two sulfur-containing amino-acids to form a peptide dimer linking sulfhydryl-to-sulfhydryl crosslinking of the present invention, wherein X₁ and X₂ are H or Me; and the linker (Y) is defined as shown. In particular embodiments, the linker (Y) can comprise homobifunctional maleimide crosslinkers, di-halide, 1,2-Bis(bromomethyl)benzene, 1,2-Bis(chloromethyl)benzene, 1,3-Bis(bromomethyl)benzene, 1,3-Bis(chlorobromoethyl)benzene, 1,4-Bis(bromomethyl)benzene, 1,4-Bis(chloromethyl)benzene 3,3′-Bis-bromomethyl-biphenyl, or 2,2′-Bis-bromomethyl-biphenyl. Certain haloacetyl crosslinkers contain an iodoacetyl or a bromoacetyl groups. In certain embodiments, these homobifunctional linkers may contain spacers, e.g., comprising a PEG or an aliphatic chain.

FIG. 6 is a chart demonstrating potency and stability data in simulated intestinal fluids (SIF) for various thioether peptide dimer compounds according to SEQ ID NO: 23 and Formula (II) in accordance with various non-limiting representative embodiment of the present invention. Lower case letters indicate D-amino acids.

FIG. 7 is a chart demonstrating potency data of various peptide monomer compounds according to Formula II in accordance with various non-limiting representative embodiments of the present invention.

FIG. 8 is a chart demonstrating stability data in simulated intestinal fluids (SIF) for various peptide monomer compounds according to Formula (II) in accordance with various non-limiting representative embodiment of the present invention.

### SEQUENCE IDENTIFIERS

The amino acid sequences listed in the accompanying sequence listing are shown using three letter code for amino acids, as defined in 37 C.F.R. 1.822. Sequences of monomer peptide molecules or the monomer subunits of dimer molecules are shown.

In the accompanying sequence listing:
SEQ ID NO: 1 shows a monomer peptide molecule or a peptide subunit of a dimer molecule representing various thioether peptides or peptide subunits of Formula (I).
SEQ ID NO: 2 shows a monomer peptide molecule or a peptide subunit of a dimer molecule representing various thioether peptides or peptide subunits of Formula (II).
SEQ ID NOs: 1-32 show amino acid sequences of illustrative thioether monomer peptides or thioether peptide subunits that are dimerized to form various thioether dimer compounds in accordance with the present invention, wherein these sequences have been substituted with an N(alpha)-Me-Arg.
SEQ ID NO: 33 shows a monomer peptide molecule or a peptide subunit of a dimer molecule representing various thioether peptides or peptide subunits of Formula (I-1).
SEQ ID NO: 34 shows a monomer peptide molecule or a peptide subunit of a dimer molecule representing various thioether peptides or peptide subunits of Formula (I-2).
SEQ ID NO: 35 shows a monomer peptide molecule or a peptide subunit of a dimer molecule representing various thioether peptides or peptide subunits of Formula (I-3).
SEQ ID NO: 36 shows a monomer peptide molecule or a peptide subunit of a dimer molecule representing various thioether peptides or peptide subunits of Formula (I-A).
SEQ ID NO: 37 shows a monomer peptide molecule or a peptide subunit of a dimer molecule representing various thioether peptides or peptide subunits of Formula (I-B).
SEQ ID NO: 38 shows a monomer peptide molecule or a peptide subunit of a dimer molecule representing various thioether peptides or peptide subunits of Formula (I-C).
SEQ ID NO: 39 shows a monomer peptide molecule or a peptide subunit of a dimer molecule representing various thioether peptides or peptide subunits of Formula (I-D).
SEQ ID NO: 40 shows a monomer peptide molecule or a peptide subunit of a dimer molecule representing various thioether peptides or peptide subunits of Formula (I-E).
SEQ ID NO: 41 shows a monomer peptide molecule or a peptide subunit of a dimer molecule representing various thioether peptides or peptide subunits of Formula (I-F).
SEQ ID NO: 42 shows a monomer peptide molecule or a peptide subunit of a dimer molecule representing various thioether peptides or peptide subunits of Formula (I-G).
SEQ ID NO: 43 shows a monomer peptide molecule or a peptide subunit of a dimer molecule representing various thioether peptides or peptide subunits of Formula (I-H).
SEQ ID NO: 44 shows a monomer peptide molecule or a peptide subunit of a dimer molecule representing various thioether peptides or peptide subunits of Formula (I-I).
SEQ ID NO: 45 shows a monomer peptide molecule or a peptide subunit of a dimer molecule representing various thioether peptides or peptide subunits of Formula (II-A).
SEQ ID NO: 46 shows a monomer peptide molecule or a peptide subunit of a dimer molecule representing various thioether peptides or peptide subunits of Formula (III).
SEQ ID NO: 47 shows a monomer peptide molecule or a peptide subunit of a dimer molecule representing various thioether peptides or peptide subunits of Formula (IV).
SEQ ID NO: 48 shows a monomer peptide molecule or a peptide subunit of a dimer molecule representing various thioether peptides or peptide subunits of Formula (A).
SEQ ID NO:49 shows a monomeric peptide molecule or a peptide subunit of a dimer molecule representing various thioether peptides or peptide subunits of Formula (V)
SEQ ID NO:50 shows a monomeric peptide molecule or a peptide subunit of a dimer molecule representing various thioether peptides or peptide subunits of Formula (VI).
SEQ ID NOs: 1, 2, 5, 6, 9-21 and 25-32 show various amino acid sequences of illustrative thioether peptides that may be acylated at their N-terminus using one of the acylating organic compounds and methods disclosed herein, including but not limited to cyclopropylacetic acid, 4-Fluorobenzoic acid, 4-fluorophenylacetic acid, 3-Phenylpropionic acid, Succinic acid, Glutaric acid, Cyclopentane carboxylic acid, 3,3,3-trifluoropropeonic acid, and 3-Fluoromethylbutyric acid.
SEQ ID NOs: 1-21 and 25-32 show amino acid sequences of illustrative monomer subunits that may be dimerized at either their N- or C-terminuses to form various thioether dimer compounds in accordance with the present invention.
SEQ ID NOs: 22-24 show amino acid sequences of monomer subunits that may be dimerized at their C-terminuses to form various thioether dimer compounds in accordance with the present invention.

### DETAILED DESCRIPTION OF THE INVENTION

As discussed above, integrins are heterodimers that function as cell adhesion molecules. The α4 integrins, α4β1 and α4β7, play essential roles in lymphocyte migration throughout the gastrointestinal tract. They are expressed on most leukocytes, including B and T lymphocytes, monocytes, and dendritic cells, where they mediate cell adhesion via binding to their respective primary ligands, namely vascular cell adhesion molecule (VCAM) and mucosal addressin cell adhesion molecule (MAdCAM). VCAM and MAdCAM differ in binding specificity, in that VCAM binds both α4β1 and α4β7, while MAdCAM is highly specific for α4β7.

The present invention relates generally to thioether peptides (e.g. peptide monomers and dimers) that have been shown to have integrin antagonist activity. In particular, the present invention relates to various peptides that form cyclized structures through thioether bonds. In certain embodiments, the thioether bonds are cyclized via covalent bonds formed between an upstream amino acid or aromatic acid group, and a downstream sulfur containing amino acid or isostere thereof. Surprisingly, thioether bonds formed when the upstream amino acid or aromatic acid group is 2-methylbenzoyl show superior potency. In some embodiments, thioether peptides comprising 2-methylbenzoyl possess superior potency as compared to thioether peptides not comprising 2-methylbenzoyl. Some aspects of the present invention contemplate that thioether peptide integrin inhibitors comprising 2-methybenzoyl show superior potency compared to non-cyclized integrin peptide inhibitors. In some embodiments, thioether peptide integrin inhibitors comprising 2-methylbenzoyl show superior potency compared to other integrin peptide inhibitors that do not include this moiety. As used herein, “superior potency” will be understood by those of skill in the art to mean a greater, higher, better, or improved potency.

Differences in the expression profiles of VCAM and MAdCAM provide the most convincing evidence of their role in inflammatory diseases. Both are constitutively expressed in the gut; however, VCAM expression extends into peripheral organs, while MAdCAM expression is confined to organs of the gastrointestinal tract. In addition, elevated MAdCAM expression in the gut has now been correlated with several gut-associated inflammatory diseases, including Crohn's disease, ulcerative colitis, and hepatitis C.

The thioether peptide monomer and dimer molecules of the invention may be used in combination with other compositions and procedures for the treatment of disease. Additionally, the monomer or dimer molecules of the present invention may be combined with pharmaceutically acceptable excipients, and optionally sustained-release matrices, such as biodegradable polymers, to form therapeutic compositions.

### Definitions

As used herein, the singular forms “a,” “and” and “the” include plural references unless the context clearly dictates otherwise.

When the term “comprising” is used herein, it is understood that the present invention also includes the same embodiments wherein the term “comprising” is substituted with “consisting essentially of” or “consisting of.”

As used in the present specification the following terms have the meanings indicated:

The term “peptide,” as used herein, refers broadly to a structure comprising a sequence of two or more amino acids joined together by peptide bonds. In particular embodiments, it refers to a sequence of two or more amino acids joined together by peptide bonds. It should be understood that this term does not connote a specific length of a polymer of amino acids, nor is it intended to imply or distinguish whether the polypeptide is produced using recombinant techniques, chemical or enzymatic synthesis, or is naturally occurring. The term “peptide”, as used generically herein, includes includes both peptide monomers and peptide dimers.

The term “monomer” as used herein may also be referred to as “peptide monomer,” “peptide monomer molecule,” or “monomer peptide.” The term “monomer” indicates a single sequence of two or more amino acids joined together by peptide bonds.

The term “dimer,” as used herein, refers broadly to a peptide comprising two monomer peptide subunits (e.g., thioether monomer peptides) that are linked at their respective C- or N-terminuses. Dimers of the present invention may include homodimers or heterodimers that function as integrin antagonists. The term “dimer” may also be referred to herein to as a “peptide dimer,” “peptide dimer molecule,” “dimer peptide,” or “dimer compound.” The term “monomer peptide subunit” may also be referred to herein as “monomer subunit,” “peptide monomer subunit,” “peptide subunit,” “peptide dimer subunit,” “dimer subunit,” “monomeric subunit,” or “subunit of a peptide dimer.”

The term “thioether,” as used herein, refers to a cyclized, covalent bond formed between an upstream amino acid or aromatic acid group, and a downstream sulfur-containing amino acid, or isostere thereof, i.e., a C—S bond.

The term “linker,” as used herein, refers broadly to a chemical structure that is capable of linking together two thioether monomer subunits to form a dimer.

The term “L-amino acid,” as used herein, refers to the “L” isomeric form of a peptide, and conversely the term “D-amino acid” refers to the “D” isomeric form of a peptide. The amino acid residues described herein are preferred to be in the “L” isomeric form, however, residues in the “D” isomeric form can be substituted for any L-amino acid residue, as long as the desired functional is retained by the peptide.

Unless otherwise indicated, the term “NH₂,” as used herein, refers to the free amino group present at the amino terminus of a polypeptide. The term “OH,” as used herein, refers to the free carboxy group present at the carboxy terminus of a peptide. Further, the term “Ac,” as used herein, refers to Acetyl protection through acylation of the N-terminus of a polypeptide. Where indicated, “NH₂” refers to a free amino group side chain of an amino acid. Where indicated, the term “Ac,” as used herein refers to acylation of an amino acid with NH₂ group.

The term “carboxy,” as used herein, refers to —CO₂H.

The term “isostere” or “isostere replacement,” as used herein, refers to any amino acid or other analog moiety having chemical and/or structural properties similar to a specified amino acid. In particular embodiments, an “isostere” or “suitable isostere” of an amino acid is another amino acid of the same class, wherein amino acids belong to the following classes based on the propensity of the side chain to be in contact with polar solvent like water: hydrophobic (low propensity to be in contact with water), polar or charged (energetically favorable contact with water). The charged amino acid residues include lysine (+), arginine (+), aspartate (−) and glutamate (−). Polar amino acids include serine, threonine, asparagine, glutamine, histidine and tyrosine. The hydrophobic amino acids include alanine, valine, leucine, isoleucine, proline, phenylalanine, tryptophane, cysteine and methionine. The amino acid glycine does not have a side chain and is hard to assign to one of the above classes. However, glycine is often found at the surface of proteins, often within loops, providing high flexibility to these regions, and an isostere may have a similar feature. Proline has the opposite effect, providing rigidity to the protein structure by imposing certain torsion angles on the segment of the polypeptide chain.

The term “cyclized,” as used herein, refers to a reaction in which one part of a polypeptide molecule becomes linked to another part of the polypeptide molecule to form a closed ring, such as by forming a thioether bond. In particular embodiments, peptide monomers and monomer subunits of peptide dimers of the present invention are cyclized via an intramolecular thioether bond.

The term “receptor,” as used herein, refers to chemical groups of molecules on the cell surface or in the cell interior that have an affinity for a specific chemical group or molecule. Binding between peptide molecules and targeted integrins can provide useful diagnostic tools.

The term “integrin-related diseases,” as used herein, refer to indications that manifest as a result of integrin binding, and which may be treated through the administration of an integrin antagonist.

The term “pharmaceutically acceptable salt,” as used herein, represents salts or zwitterionic forms of the compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

The term “N(alpha)Methylation”, as used herein, describes the methylation of the alpha amine of an amino acid, also generally termed as an N-methylation.

The term “sym methylation” or “Arg-Me-sym”, as used herein, describes the symmetrical methylation of the two nitrogens of the guanidine group of arginine. Further, the term “asym methylation” or “Arg-Me-asym” describes the methylation of a single nitrogen of the guanidine group of arginine.

The term “acylating organic compounds,” as used herein refers to various compounds with carboxylic acid functionality, which may be used to acylate the C- and/or N-termini of a peptide molecule. Non-limiting examples of acylating organic compounds include cyclopropylacetic acid, 4-Fluorobenzoic acid, 4-fluorophenylacetic acid, 3-Phenylpropionic acid, Succinic acid, Glutaric acid, Cyclopentane carboxylic acid, glutaric acid, succinic acid, 3,3,3-trifluoropropeonic acid, 3-Fluoromethylbutyric acid.

All peptide sequences are written according to the generally accepted convention whereby the a-N-terminal amino acid residue is on the left and the a-C-terminal is on the right. As used herein, the term “α-N-terminal” refers to the free α-amino group of an amino acid in a peptide, and the term “α-C-terminal” refers to the free α-carboxylic acid terminus of an amino acid in a peptide.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids are pyrrolysine (found in methanogenic organisms and other eukaryotes), selenocysteine (present in many noneukaryotes as well as most eukaryotes), and N-formylmethionine (encoded by the start codon AUG in bacteria, mitochondria and chloroplasts). “Unnatural” or “non-natural” amino acids are non-proteinogenic amino acids (i.e., those not naturally encoded or found in the genetic code) that either occur naturally or are chemically synthesized. Over 140 natural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

Generally, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. Some abbreviations useful in describing the invention are defined below in the following Table 1.

TABLE 1
Abbreviations

| Abbreviation | Definition |
|---|---|
| DIG | DIGlycolic acid (Linker) |
| Dap | Diaminopropionic acid |
| Dab | Diaminobutyric acid |
| Pen | Penicillamine |
| Sar | Sarcosine |
| Cit | Citroline |
| Cav | Cavanine |
| 4-Guan | 4-Guanidine-Phenylalanine |
| N—Me-Arg; N(alpha)Methylation | N-Methyl-Arginine |
| Ac- | Acetyl |
| 2-Nal | 2-Napthylalanine |
| 1-Nal | 1-Napthylalanine |
| Bip | Biphenylalanine |
| O-Me-Tyr | Tyrosine (O-Methyl) |
| N—Me-Lys | N-Methyl-Lysine |
| N—Me-Lys (Ac) | N—Me-Acetyl (ε) Lysine |
| Ala (3,3 diphenyle) | 3,3 diphenyl alanine |
| NH₂ | Free Amine |
| CONH₂ | Amide |
| COOH | Acid |
| Phe (4-F) | 4-Fluoro-Phenylanine |
| PEG13 | Bifunctional PEG linker with 13 PolyEthylene Glycol units |
| PEG25 | Bifunctional PEG linker with 25 PolyEthylene Glycol units |
| PEG1K | Bifunctional PEG linker with PolyEthylene Glycol Mol wt of 1000Da |
| PEG2K | Bifunctional PEG linker with PolyEthylene Glycol Mol wt of 2000Da |
| PEG3.4K | Bifunctional PEG linker with PolyEthylene Glycol Mol wt of 3400Da |
| PEG5K | Bifunctional PEG linker with PolyEthylene Glycol Mol wt of 5000Da |
| IDA | β-Ala-Iminodiacetic acid (Linker) |
| IDA-Palm | β-Ala (Palmityl)-Iminodiacetic acid |
| HPhe | Homo Phenylalanine |
| Ahx | Aminohexanoic acid |
| DIG-OH | Glycolic monoacid |
| Triazine | Amino propyl Triazine di-acid |
| Boc-Triazine | Boc-Triazine di-acid |
| Trifluorobutyric acid | Acylated with 4,4,4-Trifluorobutyric acid |
| 2-Methly-trifluorobutyric acid | acylated with 2-methy-4,4,4-Butyric acid |
| Trifluorpentanoic acid | Acylated with 5,5,5-Trifluoropentnoic acid |
| 1,4- Phenylenediacetic acid | para- Phenylenediacetic acid (Linker) |
| 1,3 - Phenylenediacetic acid | meta - Phenylenediacetic acid (Linker) |
| DTT | Dithiothreotol |
| Nle | Norleucine |
| β-HTrp | β-homoTrypophane |
| β-HPhe | β-homophenylalanine |
| Phe(4-CF₃) | 4-Trifluoromethyl Phenylalanine |
| β-Glu | 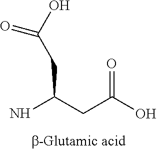 |
| β-HGlu beta-Homo-Glu | 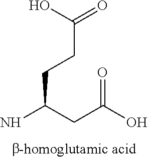 [SMILES: N[C@@H](CCC(=O)O)CC(=O)O] |
| 2-2-Indane | 2-Anninoindane-2-carboxylic acid |
| 1-1-Indane | 1-Anninoindane-1-carboxylic acid |
| Cpa | Cyclopentyl alanine |
| Orn | Ornithine |
| Aoc | 2-Amono octonoic acid |
| Cba | Cyclibutyl alanine |
| HCha | homocyclohexyl Alanine |
| Cyclobutyl | Cyclobutylalanine |
| β-HPhe, B-H-K | β-homophenylalanine |
| HLeu, homo-Leu, hK, | Homoleucine |
| Gla | Gama-Carboxy-Glutamic acid |
| Tic | 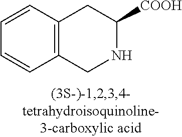 [SMILES: O=C(O)[C@@H]1Cc2ccccc2CN1] |
| Phe(4CF3) | L-Phe(4-CF₃)-OH |
| | Phe(4-trifluoromethyl |
| | 3-(4-trifluoromethyl-phenyl)propionic acid |
| Phe(2,4-diCl) | (S)-2-amino-3-(2,4-dichlorophenyl)propionic acid |
| Phe(3,4-diCl) | (S)-2-amino-3-(3,4-dichlorophenyl)propionic acid |
| Pen(═O) | Penicillamine sulfoxide |
| Aic | aminoindan-2-carboxylic acid |
| Phe(2-carbomyl) | L-2-carbamoylphenylalanine |
| Phe(3-carbomyl) | L-3-carbamoylphenylalanine |
| Phe(4-COOH) | (4-carboxy-tert-butyl)-L-phenylalanine |
| Phe(4-Ome) | (S)-4-methoxyphenylalanine |
| Phe(4tBu) | (S) -2-amino-3-(4-tert-butyl-phenyl)propionic acid |
| Phe(4-F) | 4-fluoro-L-phenylalanine |
| Glu(OMe) | L-glutamic acid g-methyl ester |
| alpha-bromobutyryl | 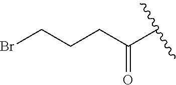 [SMILES: *C(=O)CCCBr] |
| alpha-bromopropenyl; Propionyl | 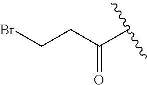 [SMILES: *C(=O)CCBr] |
| alpha-bromoisobutyryl | 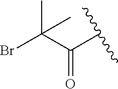 [SMILES: *C(=O)C(C)(C)Br] |
| alpha-H-E; alpha-hGlu | 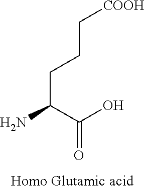 [SMILES: N[C@@H](CCCC(=O)O)C(=O)O] |
| F(2-Me) | 2-Methyl Phenylalanine |
| 4-Benzyl | 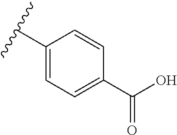 [SMILES: *c1ccc(C(=O)O)cc1] |
| 2-Benzyl | 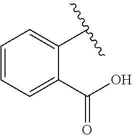 [SMILES: *c1ccccc1C(=O)O] |
| 3-Benzyl | 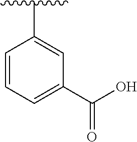 [SMILES: *c1cccc(C(=O)O)c1] |
| erythro-b-F-S | 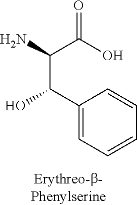 [SMILES: N[C@@H](C(=O)O)[C@@H](O)c1ccccc1] |
| Threo-b-F-S | 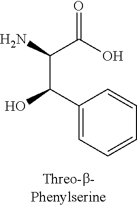 [SMILES: N[C@@H](C(=O)O)[C@H](O)c1ccccc1] |
| F(2-CF3) | 2-Trifluoromethyl-Phenylalanine |
| F(CF3) | 4-Trifluoromethyl-Phenylalinine |
| F(4-Me); 4-Me-F | 4-Methyl Phenylalanine |
| F(3-Me) | 3-Methyl Phenylalanine |
| Alpha-hGlu | HomoGlutamc acid |
| ATC | 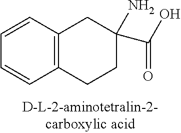 [SMILES: NC1(C(=O)O)CCc2ccccc2C1] |
| BPA | 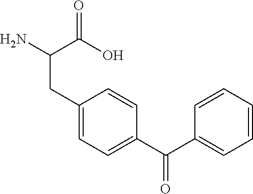 [SMILES: NC(Cc1ccc(C(=O)c2ccccc2)cc1)C(=O)O] |
| b-Me-F | 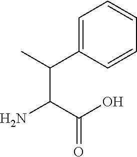 |
| β-dimethyl-F | 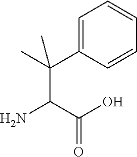 |
| 2-Chloro Benzoyl | 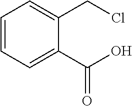 [SMILES: O=C(O)c1ccccc1CCl] |
| N—Me-E | N-Methyl Glutamic acid |
| k(Ac) | Nε-Acety -D-Lysine |
| k(PEG8) | PEG8 conjugated (Nε)-D-Lys |
| N—Me-k(Ac) | N-methyl Nε-Acetyl -D-Lysine |
| N—Me-K(Ac) | N-methyl Nε-Acetyl -Lysine |
| F(4-tBu); F(4tBu) | 4-tButyl-Phenylalanine |
| C(thioether propane) | S-CH2-CH2-CH2-S |
| 1(D-L) | D-leucine |

### Thioether Peptide Monomers and Thioether Peptide Dimers

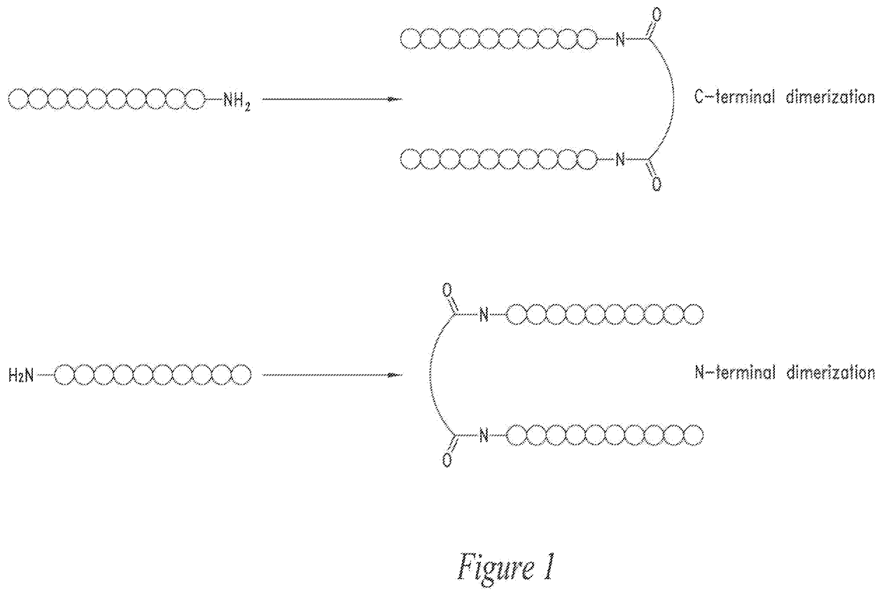

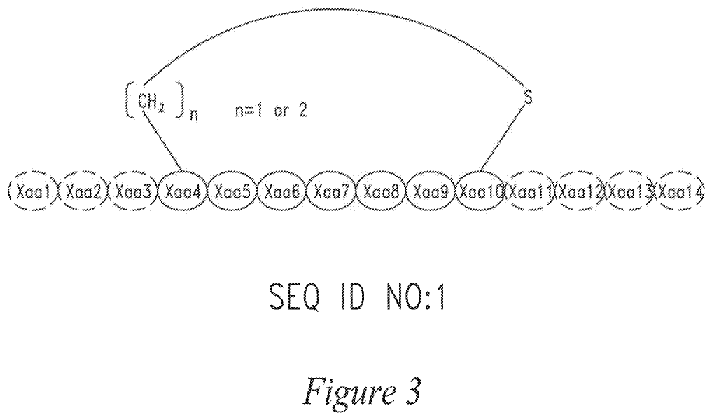

The present invention relates generally to thioether peptides that have been shown to have integrin antagonist activity. In particular, the present invention relates to various peptides that form cyclized structures through thioether bonds, e.g., intramolecular thioether bonds. Certain embodiments relate to thioether peptide monomers with integrin antagonist activity. Some embodiments relate to thioether peptide dimers with integrin antagonist activity comprising hetero- or homo-monomer thioether peptide subunits, wherein the thioether peptide subunits are linked at either their C- or N-terminuses, e.g., as shown in FIG. 1. The cyclized structure of the peptide monomers or peptide subunits have been shown to increase the potency, selectivity, and stability of the peptide molecules, as discussed below. A non-limiting, representative illustration of the cyclized structure of Formula (I) is shown in FIG. 3. In some embodiments, dimerizing the peptide monomer increases potentency, selectivity, and/or stability compared to a non-dimerized peptide.

In some instances, the monomer peptides further comprise C- and/or N-termini that comprise free amine (or both C- and N-termini that comprise free amine). Similarly, a peptide dimer may comprise one or more C- or N-termini that comprise a free amine. Thus, a user may modify either terminal end to include a modifying group such as a PEGylation, e.g., a small PEGylation (e.g. PEG4-PEG13). A user may further modify either terminal end through acylation. For example, in some instances at least one of the N- and C-terminus of a peptide molecule is acylated with an acylating organic compound selected from the group consisting of 2-Me-Trifluorobutyl, Trifluoropentyl, Acetyl, Octonyl, Butyl, Pentyl, Hexyl, Palmityl, Trifluoromethyl butyric, cyclopentane carboxylic, cyclopropylacetic, 4-fluorobenzoic, 4-fluorophenyl acetic, 3-Phenylpropionic acid. In some instances, peptide molecules of the instant invention comprise both a free carboxy terminal and a free amino terminal, whereby a user may selectively modify the peptide to achieve a desired modification. It is further understood that the C-terminal residues of the thioether peptides, e.g., thioether monomers, disclosed herein are amides or acids, unless otherwise indicated. One having skill in the art will therefore appreciate that the thioether peptides of the instant invention may be selectively modified, as desired.

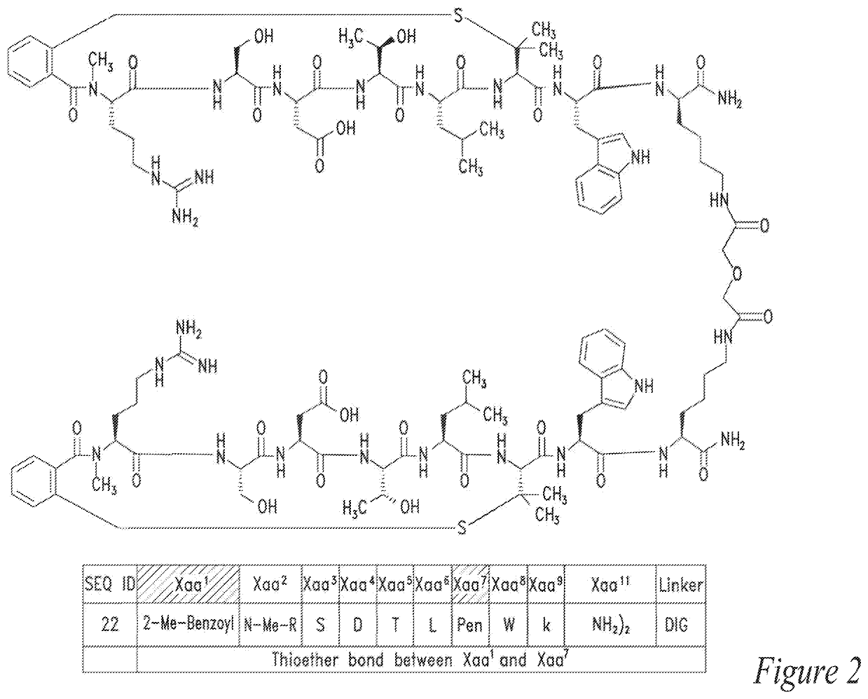

With respect to peptide dimers, it is understood that monomer subunits are dimerized to form thioether peptide dimer molecules in accordance with the present teaching and as shown generally in FIGS. 1 and 2. The monomer subunits are joined or dimerized by a suitable linker moiety, as defined herein. Some of the monomer subunits are shown having C- and N-termini that both comprise free amine. Thus, a user may modify either terminal end of the monomer subunit to eliminate either the C- or N-terminal free amine, thereby permitting dimerization at the remaining free amine. Thus, some of the monomer subunits comprise both a free carboxy or amide at C-terminal and a free amino terminal, whereby a user may selectively modify the subunit to achieve dimerization at a desired terminus. One having skill in the art will therefore appreciate that the monomer subunits of the instant invention may be selectively modified to achieve a single, specific amine for a desired dimerization.

It is further understood that the C-terminal residues of the monomer subunits disclosed herein are amides, unless otherwise indicated. Further, it is understood that dimerization at the C-terminal is facilitated by using a suitable amino acid with a side chain having amine functionality, as is generally understood in the art. In particular embodiments, a linker binds to functional amine groups in the C-terminal amino acid of each of the peptide monomer subunits to form a dimer. Regarding the N-terminal residues, it is generally understood that dimerization may be achieved through the free amine of the terminal residue, or may be achieved by using a suitable amino acid side chain having a free amine, as is generally understood in the art.

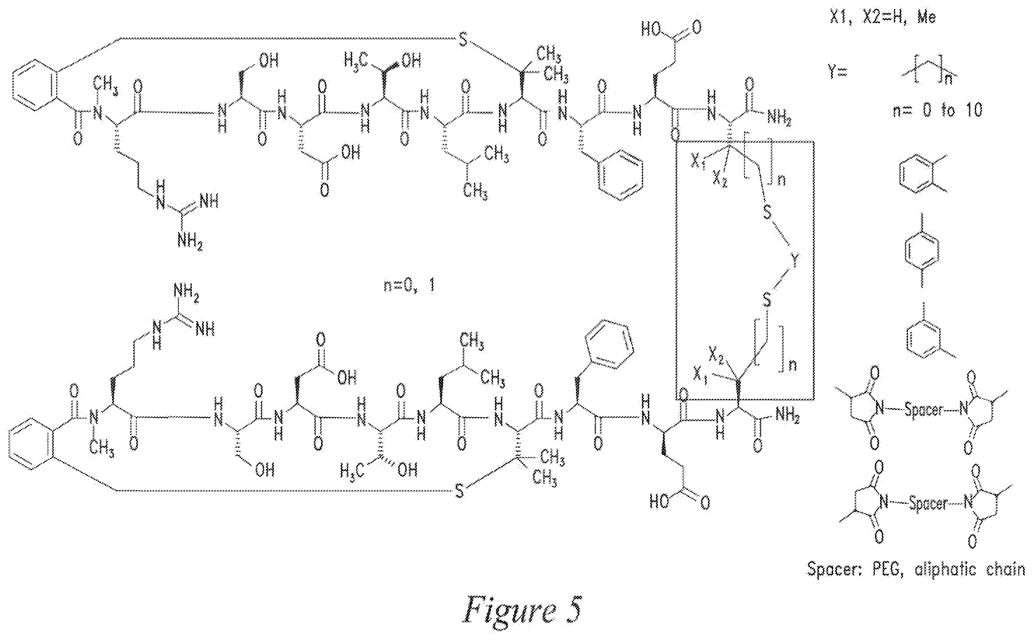

In particular embodiments, dimers are dimerized through a sulfhydryl group, e.g., via the C-terminus of each monomer subunit of the dimer. FIG. 5 shows a pair of integrin antagonist monomer subunits wherein the subunits are aligned and linked at their respective C-termini by a linker that connects two sulfur-containing amino-acids to form a peptide dimer linking sulfhydryl-to-sulfhydryl crosslinking of the present invention, wherein X₁ and X₂ are H or Me; and the linker (Y) is defined as shown. In particular embodiments, the linker (Y) can comprise homobifunctional maleimide crosslinkers, di-halide, 1,2-Bis(bromomethyl)benzene, 1,2-Bis(chloromethyl)benzene, 1,3-Bis(bromomethyl)benzene, 1,3-Bis(chloromethyl)benzene, 1,4-Bis(bromomethyl)benzene, 1,4-Bis(chloromethyl)benzene 3,3′-Bis-bromomethyl-biphenyl, or 2,2′-Bis-bromotriethyl-biphenyl. Certain haloacetyl crosslinkers contain an iodoacetyl or a bromoacetyl groups. In certain embodiments, these homobifunctional linkers may contain spacers, e.g., comprising a PEG or an aliphatic chain.

In some instances, N-terminal dimerization is proceeded by acylating the C-terminus using one of the acylating organic compounds and methods disclosed herein, including but not limited to Acetyl, cyclopropylacetic acid, 4-Fluorobenzoic acid, 4-fluorophenylacetic acid, 3-Phenylpropionic acid, Succinic acid, Glutaric acid, Cyclopentane carboxylic acid, 3,3,3-trifluoropropeonic acid, and 3-Fluoromethylbutyric acid. For example, where a C-terminal dimerization is desired, the N-terminuses of the respective monomer subunits will generally acylated prior to the C-terminuses being joined by a suitable linking moiety to provide a thioether dimer compound. Conversely, where an N-terminal dimerization is desired, the C-terminuses of the respective monomer subunits may be acylated when the C-terminus comprises a free amine, the N-terminuses being joined by a suitable linking moiety to provide a thioether dimer compound.

The peptide monomers and dimers of the instant invention, or peptide subunits thereof, may further comprise one or more terminal modifying groups. In at least one embodiment, a terminal end of a peptide is modified to include a terminal modifying group selected from the non-limiting group consisting of DIG, PEG4, PEG13, PEG25, PEG1K, PEG2K, PEG4K, PEG5K, Polyethylene glycol having molecular weight from 400 Da to 40,000 Da, PEG having a molecular weight of 40,000 Da to 80,000 Da, IDA, ADA, Glutaric acid, Succinic acid, Isophthalic acid, 1,3-phenylenediacetic acid, 1,4-phenylenediacetic acid, 1,2-phenylenediacetic acid, AADA, and suitable aliphatics, aromatics, and heteroaromatics.

In certain embodiments the N- or C-terminus of the peptide monomer or peptide dimer subunit is linked to a modifying group. In certain embodiments, the N-terminus of a peptide is modified by one to three suitable groups, e.g., as represented by Xaa¹, Xaa², and Xaa³, e.g., of Formula (I) or (I-A). Similarly, in certain embodiments, the C-terminus of a peptide is modified by a suitable group. For example, the C-terminus may be acylated. In some instances, the C-terminus further comprises a suitable linker moiety, as disclosed herein. In certain embodiments, the C-terminus comprises NH₂ or OH.

For some embodiments of peptide dimers or peptide monomers described herein, any of Xaa¹-Xaa⁵, Xaa⁷-Xaa⁹, and Xaa¹¹-Xaa¹² are N(alpha)Methylated. Xaa⁵ may further be Arg-Me-sym or Arg-Me-asym, and Xaa¹¹ may be O-Me-Tyr, N-Me-Lys(Ac), or 4-Me-Phe. The N-terminus may further be acylated. In some instances, any of Xaa¹-Xaa⁴, and Xaa¹¹-Xaa¹⁴ are acylated. For example, in some instances one or more residues at positions Xaa⁸-Xaa¹¹ are acylated with an acylating organic compound selected from the group consisting of 2-Me-Trifluorobutyl, Trifluoropentyl, Acetyl, Octonyl, Butyl, Pentyl, Hexyl, Palmityl, Trifluoromethyl butyric, cyclopentane carboxylic, cyclopropylacetic, 4-fluorobenzoic, 4-fluorophenyl acetic, and 3-Phenylpropionic acid. In some instances one or more residues at positions Xaa¹-Xaa⁴, and Xaa¹¹-Xaa¹⁴ are acylated with an acylating organic compound selected from the group consisting of 2-me-Trifluorobutyl, Trifluoropentyl, Acetyl, Octonyl, Butyl, Pentyl, Hexyl, Palmityl, Lauryl, Oleoyl, and Lauryl, Trifluoromethyl butyric, cyclopentane carboxylic, cyclopropylacetic, 4-fluorobenzoic, 4-fluorophenyl acetic, 3-Phenylpropionic, tetrahedro-2H-pyran-4carboxylic, succinic acid, and glutaric acid. In some instances, small PEG (e.g., PEG4-PEG13) is used as spacer before acylations.

In some embodiments of the peptide dimers, peptide dimer subunits or peptide monomers described herein, the N-terminus further comprises a suitable linker moiety or other modifying group. In some embodiments of peptide monomers described herein, the N-terminus may further be acylated.

Non-limiting examples of terminal modifying groups are provided in Table 2.

TABLE 2
Illustrative Terminal Modifying Groups

| Abbreviation | Description | Structure |
|---|---|---|
| DIG | DIGlycolic acid, | 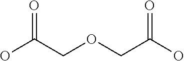 |
| PEG4 | Bifunctional PEG linker with 4 PolyEthylene Glycol units | 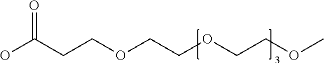 |
| PEG13 | PEG with 13 PolyEthylene Glycol units | 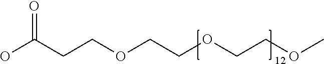 |
| PEG25 | PEG with 25 PolyEthylene Glycol units | 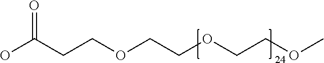 |
| PEG1K | PolyEthylene Glycol Mol wt of 1000Da | |
| PEG2K | PolyEthylene Glycol Mol wt of 2000Da | |
| PEG3.4K | PolyEthylene Glycol Mol wt of 3400Da | |
| PEG5K | PolyEthylene Glycol Mol wt of 5000Da | |
| DIG | DIGlycolic acid, | 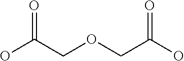 |
| IDA | β-Ala-Iminodiacetic acid | 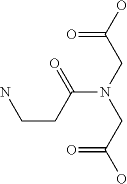 [SMILES: NCCC(=O)N(CC(=O)O)CC(=O)O] |
| Boc-IDA | Boc-β-Ala-Iminodiacetic acid | 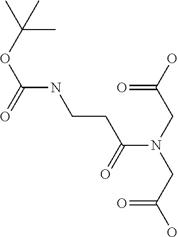 [SMILES: CC(C)(C)OC(=O)NCCC(=O)N(CC(=O)O)CC(=O)O] |
| Ac-IDA | Acetyl-β-Ala-Iminodiacetic acid | 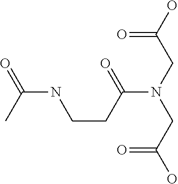 [SMILES: CC(=O)NCCC(=O)N(CC(=O)O)CC(=O)O] |
| GTA | Glutaric acid | 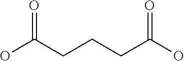 |
| PMA | Pemilic acid | 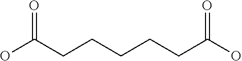 [SMILES: O=C(O)CCCCCC(=O)O] |
| AZA | Azelaic acid | 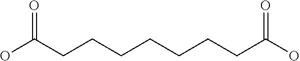 [SMILES: O=C(O)CCCCCCCC(=O)O] |
| DDA | Dodecanedioic acid | 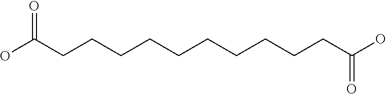 [SMILES: O=C(O)CCCCCCCCCCC(=O)O] |
| ADA | Amino diacetic acid | 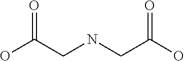 |
| AADA | n-Acetyl amino acetic acid | 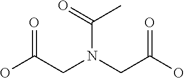 |
| PEG4-Biotin | PEG4-Biotin (Product number 10199, QuantaBioDesign) | 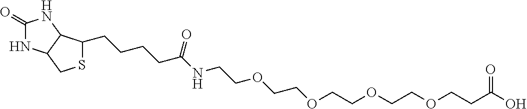 [SMILES: O=C(O)CCOCCOCCOCCOCCNC(=O)CCCCC1SCC2NC(=O)NC21] |

The linker moieties of the instant invention may include any structure, length, and/or size that is compatible with the teachings herein. In at least one embodiment, a linker moiety is selected from the non-limiting group consisting of DIG, PEG4, PEG4-biotin, PEG13, PEG25, PEG1K, PEG2K, PEG3.4K, PEG4K, PEG5K, IDA, ADA, Boc-IDA, Glutaric acid, Isophthalic acid, 1,3-phenylenediacetic acid, 1,4-phenylenediacetic acid, 1,2-phenylenediacetic acid, Triazine, Boc-Triazine, IDA-biotin, PEG4-Biotin, AADA, suitable aliphatics, aromatics, heteroaromatics, and polyethylene glycol based linkers having a molecular weight from approximately 400 Da to approximately 40,000 Da or approximately 40,000 Da to approximately 80,000 Da.

When the linker is IDA, ADA or any linker with free amine it can be acylated with acylating organic compound selected from the group consisting of 2-me-Trifluorobutyl, Trifluoropentyl, Acetyl, Octonyl, Butyl, Pentyl, Hexyl, Palmityl, Lauryl, Oleoyl, Lauryl, Trifluoromethyl butyric, cyclopentane carboxylic, cyclopropylacetic, 4-fluorobenzoic, 4-fluorophenyl acetic, 3-Phenylpropionic, tetrahedro-2H-pyran-4carboxylic, succinic acid, and glutaric acid, straight chain aliphatic acids with 10 to 20 carbon units, cholic acid and other bile acids. In some instances small PEG (PEG4-PEG13), Glu, or Asp is used as spacer before acylations.

In certain embodiments, the linker connects two monomeric subunits by connecting two sulfur containing C- or N-terminal amino acids. In some embodiments, the two sulfur containing amino acids are connected by a linker comprising a di-halide, an aliphatic chain, or a PEG. In certain embodiments, the linker connects two monomeric subunits by connecting sulfur containing C-terminal amino acids at the C-terminus of each monomer subunit. In some embodiments, the two sulfur containing amino acids are connected by a linker comprising homobifunctional maleimide crosslinkers, di-halide, 1,2-Bis(bromomethyl)benzene, 1,2-Bis(chloromethyl)benzene, 1,3-Bis(bromomethyl)benzene, 1,3-Bis(chloromethyl)benzene, 1,4 Bis(bromomethyl)benzene, 1,4-Bis(chloromethyl)benzene, 3,3′-bis-bromomethyl-biphenyl, or 2,2-bis-bromomethyl-biphenyl. Particular haloacetyl crosslinkers contain an iodoacetyl or a bromoacetyl group. These homobifunctional linkers may contain spacers comprising PEG or an aliphatic chain.

Non-limiting examples of suitable linker moieties are provided in Table 3.

TABLE 3
Illustrative Linker Moieties

| Abbrivation | Discription | Structure |
|---|---|---|
| DIG | DIGlycolic acid, | 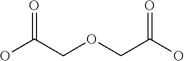 |
| PEG4 | Bifunctional PEG linker with 4 PolyEthylene Glycol units | 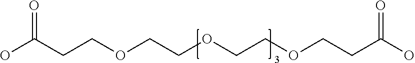 |
| PEG13 | Bifunctional PEG linker with 13 PolyEthylene Glycol units | 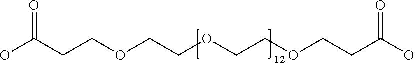 |
| PEG25 | Bifunctional PEG linker with 25 PolyEthylene Glycol units | 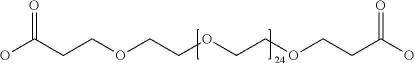 |
| PEG1K | Bifunctional PEG linker | |
| | with PolyEthylene Glycol | |
| | Mol wt of 1000Da | |
| PEG2K | Bifunctional PEG linker | |
| | with PolyEthylene Glycol | |
| | Mol wt of 2000Da | |
| PEG3.4K | Bifunctional PEG linker | |
| | with PolyEthylene Glycol | |
| | Mol wt of 3400Da | |
| PEG5K | Bifunctional PEG linker | |
| | with PolyEthylene Glycol | |
| | Mol wt of 5000Da | |
| DIG | DIGlycolic acid, | 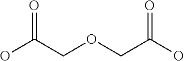 |
| IDA | β-Ala-Iminodiacetic acid | 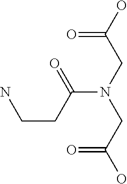 [SMILES: NCCC(=O)N(CC(=O)O)CC(=O)O] |
| Boc-IDA | Boc-β-Ala-Iminodiacetic acid | 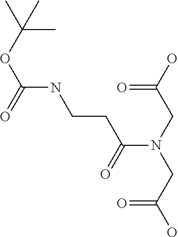 [SMILES: CC(C)(C)OC(=O)NCCC(=O)N(CC(=O)O)CC(=O)O] |
| Ac-IDA | Ac-β-Ala-Iminodiacetic acid | 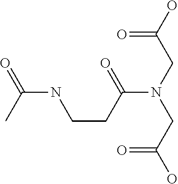 [SMILES: CC(=O)NCCC(=O)N(CC(=O)O)CC(=O)O] |
| IDA-Palm | Palmity1-β-Ala-Iminodiacetic acid | 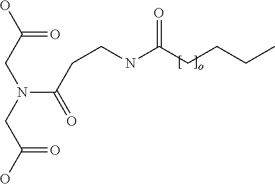 |
| GTA | Glutaric acid | 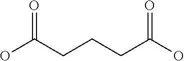 |
| PMA | Pemilic acid | 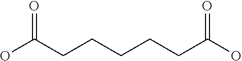 [SMILES: O=C(O)CCCCCC(=O)O] |
| AZA | Azelaic acid | 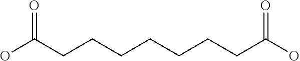 [SMILES: O=C(O)CCCCCCCC(=O)O] |
| DDA | Dodecanedioic acid | 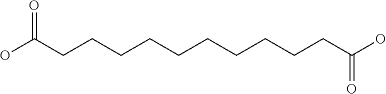 [SMILES: O=C(O)CCCCCCCCCCC(=O)O] |
| IPA | Isopthalic aicd | 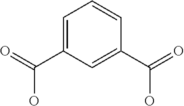 [SMILES: O=C(O)c1cccc(C(=O)O)c1] |
| 1,3-PDA | 1,3- Phenylenediacetic acid | 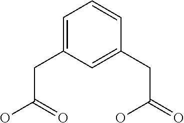 [SMILES: O=C(O)Cc1cccc(CC(=O)O)c1] |
| 1,4-PDA | 1,4- Phenylenediacetic acid | 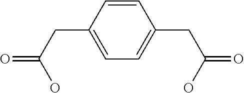 [SMILES: O=C(O)Cc1ccc(CC(=O)O)cc1] |
| 1,2-PDA | 1,2 - Phenylenediacetic acid | 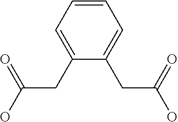 [SMILES: O=C(O)Cc1ccccc1CC(=O)O] |
| Triazine | Amino propyl Triazine di-acid | 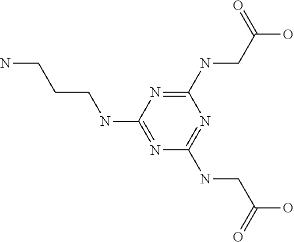 [SMILES: NCCCNc1nc(NCC(=O)O)nc(NCC(=O)O)n1] |
| Boc- Triazine | Boc-Triazine di-acid | 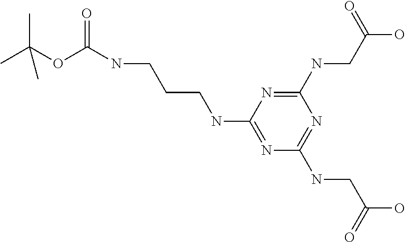 [SMILES: CC(C)(C)OC(=O)NCCCNc1nc(NCC(=O)O)nc(NCC(=O)O)n1] |
| ADA | Amino diacetic acid | 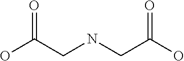 |
| AADA | n-Acetyl amino acetic acid | 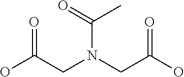 |
| PEG4- Biotin | PEG4-Biotin (Product number 10199, QuantaBioDesign) | 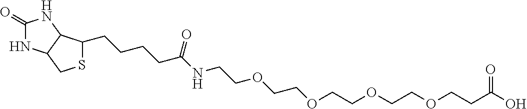 [SMILES: O=C(O)CCOCCOCCOCCOCCNC(=O)CCCCC1SCC2NC(=O)NC21] |
| 1,4 BMB | 1,4-Bis(halo-momethyl)benzene | 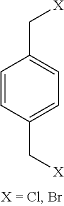 |
| 1,2 BMB | 1,2-Bis(halo-momethyl)benzene | 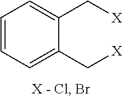 |
| 1,3 BMB | 1,3-Bis(halo-momethyl)benzene, | 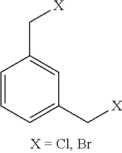 |
| 1,3 BMBip | 3,3′-Bis-Halomethyl-Biphenyl | 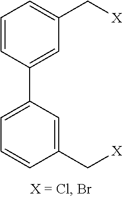 |
| IDA-Biotin | N-Biotin-β-Ala-Iminodiacetic acid | 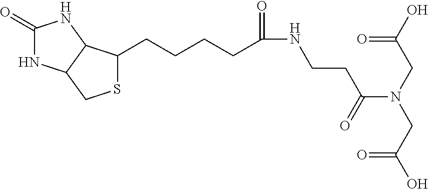 [SMILES: O=C(O)CN(CC(=O)O)C(=O)CCNC(=O)CCCCC1SCC2NC(=O)NC21] |
| 2,2 BMBip | 2,2′-Bis-Halomethyl-Biphenyl | 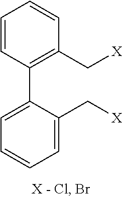 |
| BMal | Bis-Mal-dPEG | 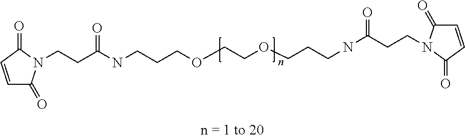 |

The present invention further includes various thioether peptide monomers or thioether peptide dimers (and subunits thereof) that have been substituted with various modified amino acids, including but not limited to any of those shown in Table 1 or described herein. For example, some peptides include Dab, Dap, Pen, Sar, Cit, Cav, HLeu, 2-Nal, D-1-Nal, D-2-Nal, Bip, O-Me-Tyr, β-HTrp, β-HPhe, Phe (4-CF3), 2-2-Indane, 1-1-Indane, Cyclobutyl, β-HPhe, HLeu, Gla, HPhe, 1-Nal, Nle, homo amino acids, D-amino acids, 3-3-diPhe, cyclobutyl-Ala, HCha, Phe(4-NH2), Bip, β-HPhe, β-Glu, 4-guan, and various N-methylated amino acids. One having skill in the art will appreciate that additional substitutions may be made to achieve similar desired results, and that such substitutions are within the teaching and spirit of the present invention. In certain embodiments, any of the peptides, e.g. peptide dimers and peptide monomer or subunits thereof, described herein or shown in the sequence listing or accompanying figures further comprises one or more amino acid substitutions, e.g., in certain embodiments, one or more amino acid residues is substituted with Dab, Dap, Pen, Sar, Cit, Cav, HLeu, 2-Nal, D-1-Nal, D-2-Nal, Bip, O-Me-Tyr, β-HTrp, β-HPhe, Phe (4-CF3), 2-2-Indane, 1-1-Indane, Cyclobutyl, β-HPhe, HLeu, Gla, HPhe, 1-Nal, Nle, homo amino acids, D-amino acids, 3-3-diPhe, cyclobutyl-Ala, HCha, Phe(4-NH2), Bip, β-HPhe, β-Glu, 4-guan, or an N-methylated amino acid, such as, e.g., N-methyl-Arg.

As used herein, “Xaa¹¹ can stand for one or more of any naturally occurring amino acids, unnatural amino acids, modified amino acids, and/or non-naturally occurring amino acids, including D- and L-amino acids, aminoacyl residues or any chemical moiety capable of substituting and amino acid position. In some embodiments, Xaa designates that more than one amino acid, aminoacyl residue, or chemical residency may occupy a given position in the peptide. In particular embodiments, Xaa designates that a single non-naturally occurring, unnatural, or modified amino acid, or an aminoacyl residue or a chemical moiety (e.g., a 2-methylbenzoyl moiety) occupies a given position in the polypeptide.

One aspect of the present invention relates to a thioether peptide monomer, a thioether peptide dimer, or a thioether subunit of a dimer molecule comprising the structure according to Formula (I):
Xaa¹-Xaa²-Xaa³-Xaa⁴-Xaa⁵-Xaa⁶-Xaa⁷-Xaa⁸-Xaa⁹-Xaa¹⁰-Xaa¹¹-Xaa¹²-Xaa¹³-Xaa¹⁴ (Formula (I); SEQ ID NO: 388; FIG. 1), or a pharmaceutically acceptable salt thereof, wherein the peptide monomer or one or both subunits of the thioether peptide dimer comprises a thioether bond between Xaa⁴ and Xaa¹⁰ to provide a cyclized structure, and wherein:
Xaa¹ is absent, or selected from the group consisting of any naturally occurring amino acid, a suitable isostere, and corresponding D-amino acids;
Xaa² is absent, or Xaa² is selected from the group consisting of any naturally occurring amino acid, a suitable isostere, and corresponding D-amino acids;
Xaa³ is absent, or Xaa³ is selected from the group consisting of any naturally occurring amino acid, a suitable isostere, and corresponding D-amino acids;
Xaa⁴ is an amino acid residue having a side chain with one or two carbons, and forming a thioether bond with Xaa¹⁰;
Xaa⁵ is selected from the group consisting of N(alpha)-Me-Arg, Arg, HArg, Dap, Dab, Arg-Me-sym, Arg-Me-asym, 4-Guan, Cit, Cav, and suitable isostere replacements;
Xaa⁶ is selected from the group consisting of Ser, Gly, and suitable isostere replacements;
Xaa⁷ is selected from the group consisting of Asp, N-Me-Asp, Asp(OMe), D-Asp, and a suitable isostere replacements;
Xaa⁸ is selected from the group consisting of Thr, Gln, Ser, Asp, Pro, Gly, His, Ala, Ile, Phe, Lys, Arg, Asn, Glu, Val, Tyr, Trp, Leu, Met, and N-Methyl amino acids including N-Me-Thr;
Xaa⁹ is selected from the group consisting of Gln, Asn, Asp, Pro, Gly, Ala, Phe, Leu, Glu, Ile, Val, HLeu, n-Butyl Ala, n-Pentyl Ala, n-Hexyl Ala, Nle, cyclobutyl-Ala, N-Me-Leu, and suitable isostere replacements;
Xaa¹⁰ is selected from the group consisting of Cys, N-Me-Cys, D-Cys, HCys, Pen, and D-Pen;
Xaa¹¹ is absent, or selected from the group consisting of Gly, Gln, Asn, Asp, Ala, Ile, Leu, Val, Met, Thr, Lys, Trp, Tyr, His, Glu, Ser, Arg, Pro, Phe, Sar, 1-Nal, 2-Nal, D-1-Nal, D-2-Nal, HPhe, D-Phe, D-Tyr, Phe(4-F), O-Me-Tyr, dihydro-Trp, Dap, Dab, Dab(Ac), Orn, D-Om, N-Me-Orn, N-Me-Dap, D-Dap, D-Dab, Bip, Ala(3,3diphenyl), Biphenyl-Ala, aromatic ring substituted Phe, aromatic ring substituted Trp, aromatic ring substituted His, hetero aromatic amino acids, N-Me-Lys, N-Me-Lys(Ac), 4-Me-Phe, and corresponding D-amino acids and suitable isostere replacements;
Xaa¹² is absent, or Xaa¹² is selected from the group consisting of Glu, Amide, Lys, COOH, Gln, Pro, Gly, His, Ala, Ile, Phe, Arg, Leu, Val, Tyr, Trp, Met, Gla, Ser, Asn, D-Glu, β-HGlu, 2-Nal, 1-Nal, D-Asp, Bip, β-HPhe, β-Glu, D-Tyr, D-Phe, D-Lys, Dap, Dab, Orn, D-Orn, N-Me-Orn, N-Me-Dap, N-Me-Dab, N-Me Lys, D-Dap, D-Dab, suitable isosteres, and corresponding D-amino acids;
Xaa¹³ may be absent, or Xaa³ is selected from the group consisting of Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, N-Me-Lys, D-N-Me-Lys, D-Dap, D-Dab, COOH, CONH₂, suitable isosteres, and corresponding D-amino acids;
Xaa¹⁴ is absent, or Xaa¹⁴ is selected from the group consisting of Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, N-Me-Lys, D-N-Me-Lys, D-Dap, D-Dab, COOH, CONH₂, suitable isosteres, corresponding D-amino acids, and corresponding N-Methyl amino acids.

In some embodiments of Formula (I), Xaa⁴ is selected from the group consisting of modified Ser, modified HSer, a suitable isostere, and corresponding D-amino acids and capable of forming a thioether bond with Xaa¹⁰. In other instances, Xaa⁴ is an aliphatic acid having from one to four carbons and capable of forming a thioether bond with Xaa¹⁰. In some instances, Xaa⁴ is a five- or six-membered alicyclic acid having a modified 2-methyl group that forms a thioether bond with Xaa¹⁰. In some embodiments, Xaa⁴ is acetyl, propionyl, alpha-bromoisobutyryl, or 2-methylbenzoyl. In particular embodiments, Xaa⁴ is a 2-methylbenzoyl moiety that forms a thioether bond with Xaa¹⁰.

The present invention also includes peptides comprising the same structure as shown in Formula (I) or any of the other formulas or tables described herein, but where the thioether bond is in the reverse orientation. In such embodiments of the invention, it may generally be considered that the amino acid residues or other chemical moieties shown at Xaa⁴ are instead present at Xaa¹⁰, and the amino acid residues shown at Xaa¹⁰ are instead present at Xaa⁴, i.e., the amino acid residue comprising the sulfur of the resulting thioether bond is located at Xaa⁴ instead of Xaa¹⁰, and the amino acid residue or other moiety having a carbon side chain capable of forming a thioether bond with Xaa⁴ is located at Xaa¹⁰. In this reverse orientation, however, the amino acid or chemical moiety at position Xaa¹⁰ is one that comprises a free amine. For example, in particular embodiments, the amino acid at Xaa¹⁰ is a protected homoserine, such as, e.g., homoserine (OTBDMS). Thus, in particular reverse orientation embodiments of Formula (I), Xaa¹⁰ is an amino acid residue having a side chain with one or two carbons, and forming a thioether bond with Xaa⁴, and Xaa⁴ is selected from the group consisting of Cys, N-Me-Cys, D-Cys, HCys, Pen, and D-Pen. Specific examples of amino acid residues and other chemical moieties present at corresponding positions of other formulas and tables are described herein.

In certain embodiments, a thioether peptide dimer comprises two peptide monomer subunit of Formula (I), wherein these subunits are linked via a linker moiety through their C- or N-termini. In one embodiment, they are linked via both their C-termini.

In another aspect, the present invention includes a thioether peptide molecule (e.g. a peptide monomer, peptide dimer, or a peptide dimer subunit) comprising the structure according to Formula (I-1) (SEQ ID NO: 389):
Xaa¹-Xaa²-Xaa³-Xaa⁴-Xaa⁵-Xaa⁶-Xaa⁷-Xaa⁸-Xaa⁹-Xaa¹⁰-Xaa¹¹-Xaa¹²-Xaa¹³-Xaa¹⁴ (Formula (I-1)), or a pharmaceutically acceptable salt thereof, wherein the peptide comprises a thioether bond between Xaa⁴ and Xaa¹⁰, and wherein:
Xaa¹ is absent, or Xaa¹ is any amino acid;
Xaa² is absent, or Xaa² is any amino acid;
Xaa³ is absent, or Xaa³ is any amino acid;
Xaa⁴ is an amino acid, aliphatic acid, alicyclic acid, or modified 2-methyl aromatic acid having a side chain with one or two carbons, and capable of forming a thioether bond with Xaa¹⁰;
Xaa⁵ is selected from the group consisting of N(alpha)-Me-Arg, Arg, HomoArg, Dap, Dab, Arg-Me-sym, Arg-Me-asym, 4-Guan, Cit, Cav, N-Me-Lys, Phe(4-quanidino), Phe(4-carbamoyl amino), Phe(4-NH₂), N-Me-HomoArg, Tyr, His, and suitable isostere replacements;
Xaa⁶ is selected from the group consisting of Ser, Gly, Thr, Ile, and suitable isostere replacements; wherein if Formula (I-1) is directed to a dimer peptide subunit, then in some embodiments, Xaa⁶ is selected from the group consisting of Ser, Gly, Thr, and suitable isostere replacements;
Xaa⁷ is selected from the group consisting of Asp, N-Me-Asp, Asp(OMe), D-Asp, and suitable isostere replacements;
Xaa⁸ is selected from the group consisting of Thr, Gln, Ser, Asp, Pro, Gly, His, Ala, Ile, Phe, Lys, Arg, Asn, Glu, Val, Tyr, Trp, Leu, Met, HomoLeu, Nle, and N-Methyl amino acids including N-Me-Thr;
Xaa⁹ is selected from the group consisting of Gln, Asn, Asp, Pro, Gly, Ala, Phe, Leu, Glu, Ile, Val, HLeu, n-Butyl Ala, n-Pentyl Ala, n-Hexyl Ala, Nle, cyclobutyl-Ala, Cpa, Aoc, N-Me-Leu, and suitable isostere replacements;
Xaa¹⁰ is selected from the group consisting of Cys, N-Me-Cys, D-Cys, HCys, Pen, D-Pen, and Pen(═O);
Xaa¹¹ is absent, or Xaa¹¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Phe (2-carbomyl), Tyr(Me), HomoPhe, N-Me-Phe, N-Me-Tyr, Ser, Sar, Dihydro Trp, Ile, Leu, Ser, Arg, Thr, Sar, and Ser, aromatic amino acids, substituted aromatic amino acids, Gly, Gln, Asn, Asp, Ala, Ile, Leu, Val, Met, Thr, Lys, Trp, Tyr, His, Glu, Ser, Arg, Pro, Phe, Sar, 1-Nal, 2-Nal, D-1-Nal, D-2-Nal, HPhe, D-Phe, D-Tyr, Phe(4-F), O-Me-Tyr, dihydro-Trp, Dap, Dab, Dab(Ac), Orn, D-Orn, N-Me-Orn, N-Me-Dap, D-Dap, D-Dab, Bip, Ala(3,3diphenyl), Biphenyl-Ala, aromatic ring substituted Phe, aromatic ring substituted Trp, aromatic ring substituted His, hetero aromatic amino acids, N-Me-Lys, N-Me-Lys(Ac), 4-Me-Phe, Phe(4tBu), Phe(4-OMe), Phe(4-COOH), Phe(2-carbomyl), Phe(3-carbomyl), Phe(CF3), Phe(2,4-diCl), Phe(3,4-diCl), Aic, N-Me-Tyr, N-Me-Phe, Tic, Phe(4CF3), and corresponding D-amino acids and suitable isostere replacements;
Xaa¹² is absent, or Xaa¹² is selected from the group consisting of aromatic amino acids, substituted aromatic amino acids, Glu, D-Glu, HomoGlu, Beta-Homo-Glu, Asp, D-HomoGlu, Amide, Lys, COOH, CONH₂, Gln, Pro, Gly, His, Ala, Ile, Phe, Arg, Leu, Val, Tyr, Trp, Met, Gla, Ser, Asn, D-Glu, β-HGlu, 2-Nal, 1-Nal, D-Asp, Bip, β-HPhe, β-Glu, D-Tyr, D-Phe, D-Lys, Dap, Dab, Orn, D-Orn, N-Me-Orn, N-Me-Dap, N-Me-Dab, N-Me Lys, D-Dap, D-Dab, D-His, F(4-COOH), Tic, D-Trp, D-Leu, D-Arg, D-Thr, suitable isosteres, and corresponding D-amino acids;
Xaa¹³ is absent, or Xaa¹³ is any amino acid; and
Xaa¹⁴ is absent or any amino acid; wherein in certain embodiments, if Formula (I-1) is directed to a peptide dimer or subunit thereof, then Xaa¹⁴ is absent or selected from the group consisting of: any amino acid with an amine side chain, Lys, D-Lys, N-Me-Lys, D-N-Me-Lys, Orn, N-Me-Orn, Dab, N-Me-Dab, Dap, N-Me-Dap, Homo-Lys, D-Dap, D-Dab, D-Orn, Gln, Pro, Gly, His, Ala, Ile, Phe, Lys, Arg, Leu, Val, Tyr, Trp, Met, Glu, Ser, Asn, Gla, Cys, HomoCys, COOH, CONH₂, suitable isosteres, corresponding D-amino acids, and corresponding N-Methyl amino acids.

In some embodiments, Xaa⁴ is acetyl, propionyl, alpha-bromoisobutyryl, or 2-methylbenzoyl. In particular embodiments, Xaa⁴ is 2-methylbenzoyl. In particular embodiments, Xaa⁴ is 2-methylbenzoyl.

In certain embodiments, a thioether peptide dimer comprises two peptide monomer subunit of Formula (I-1), wherein these subunits are linked via a linker moiety through their C- or N-termini. In one embodiment, they are linked via both their C-termini.

In particular embodiments, Formula (I-1) is directed to a peptide monomer or a peptide dimer (or subunit thereof), and Xaa⁷ is selected from the group consisting of Asp, N-Me-Asp, and D-Asp.

In certain embodiments, Xaa¹³ is present and selected from the group consisting of Gln, Pro, Gly, His, Ala, Ile, Phe, Lys, Arg, Leu, Val, Tyr, Trp, Met, Glu, Ser, Asn, Gla, Dap, Dab, Orn, D-Om, D-Lys, N-Me-Orn, N-Me-Dap, N-Me-Dab, N-Me-Lys, D-N-Me-Lys, D-Dap, D-Dab, COOH, CONH₂, suitable isosteres, and corresponding D-amino acids.

In certain embodiments, Xaa¹⁴ is present. In certain embodiments, Xaa¹⁴ is selected from the group consisting of Gln, Pro, Gly, His, Ala, Ile, Phe, Lys, Arg, Leu, Val, Tyr, Trp, Met, Glu, Ser, Asn, Gla, Dap, Dab, Orn, D-Om, D-Lys, N-Me-Orn, N-Me-Dap, N-Me-Dab, N-Me-Lys, D-N-Me-Lys, D-Dap, D-Dab, COOH, CONH₂, suitable isosteres, corresponding D-amino acids, and corresponding N-Methyl amino acids. In certain embodiments, Xaa¹⁴ is D-Lys, N-Me-Lys, Dap, or Dab. In particular embodiments, Formula (I-1) is directed to a dimer peptide or subunit thereof and Xaa¹⁴ is Cys, HomoCys or Pen. In certain embodiments, Xaa¹² and Xaa¹³ are absent, and Xaa¹⁴ is D-Lys, N-Me-Lys, Dap, or Dab. In certain embodiments, Xaa¹³ is absent, and Xaa¹⁴ is D-Lys, N-Me-Lys, Dap, or Dab. In some embodiments, Xaa¹², Xaa¹³ and Xaa¹⁴ are absent.

In certain embodiments, the amino acid immediately carboxyl to Xaa¹⁰ is an aromatic amino acid.

In particular embodiments, Formula I-1 is directed to a peptide monomer, dimer, or subunit thereof, and any one or more of Xaa¹, Xaa² or Xaa³ is selected from the group consisting of any naturally occurring amino acid, a suitable isostere, and corresponding D-amino acids

In particular embodiments, Xaa⁴ is an amino acid residue having a side chain with one or two carbons.

In particular instances, a peptide monomer, dimer, or subunit thereof of any of the Formula and peptides described herein comprises Xaa⁴, where Xaa⁴ is selected from the group consisting of modified Ser, modified HomoSer (e.g., Homo-Ser-Cl), a suitable isostere, and corresponding D-amino acids. In other instances, Xaa⁴ is an aliphatic acid having from one to four carbons and forming a thioether bond with Xaa¹⁰. In some instances, Xaa⁴ is a five- or six-membered alicyclic acid having a modified 2-methyl group that forms a thioether bond with Xaa¹⁰. In some embodiments, Xaa⁴ is a 2-methylbenzoyl moiety.

For some embodiments, at least one of Xaa¹, Xaa², Xaa³, Xaa⁵, Xaa⁷, Xaa⁸, Xaa⁹, Xaa¹¹, Xaa¹², Xaa¹³ and Xaa¹⁴ is N(alpha)Methylated. In some instances, at least one of Xaa¹, Xaa², Xaa³, Xaa⁴, Xaa¹¹, Xaa¹², Xaa¹³ and Xaa¹⁴ are acylated. For example, in some instances one or more residues at positions Xaa¹-Xaa⁴, and Xaa¹¹-Xaa¹⁴ are acylated with an acylating organic cisestyl, Hexyl, Palmityl, Lauryl, Oleoyl, and Lauryl, Trifluoromethyl butyric, cyclopentane carboxylic, cyclopropylacetic, 4-fluorobenzoic, 4-fluorophenyl acetic, 3-Phenylpropionic, tetrahedro-2H-pyran-4carboxylic, succinic acid, and glutaric acid. In some instances, small PEG (e.g., PEG4-PEG13) is used as spacer before acylations. The present invention also includes reverse order thioether bond embodiments of Formula (I-1), wherein Xaa¹⁰ is an amino acid, aliphatic acid, alicyclic acid, or modified 2-methyl aromatic acid having a side chain with one or two carbons, and capable of forming a thioether bond with Xaa⁴; and Xaa⁴ is selected from the group consisting of Cys, N-Me-Cys, D-Cys, HCys, Pen, D-Pen, and Pen(═O). In this reverse orientation, the amino acid or chemical moiety at position Xaa¹⁰ is one that comprises a free amine. One example of an amino acid or chemical moiety that provides a free amine is homoserine or a protected homoserine, e.g., homserine(OTBDMS).

In one aspect, the present invention provides a peptide (e.g. a peptide monomer, a peptide dimer, or a peptide dimer subunit) comprising the structure according to Formula (I-2)(SEQ ID NO: 34):
Xaa¹-Xaa²-Xaa³-Xaa⁴-Xaa⁵-Xaa⁶-Xaa⁷-Xaa²-Xaa⁹-Xaa¹⁰-Xaa¹¹-Xaa¹²-Xaa¹³-Xaa¹⁴ (Formula I-2), or a pharmaceutically acceptable salt thereof, wherein the peptide molecule comprises a thioether bond between Xaa⁴ and Xaa¹⁰, and wherein
Xaa¹ is absent, or Xaa¹ is selected from the group consisting of any naturally occurring amino acid, a suitable isostere, and corresponding D-amino acids;
Xaa² is absent, or Xaa² is selected from the group consisting of any naturally occurring amino acid, a suitable isostere, and corresponding D-amino acids;
Xaa³ is absent, or Xaa³ is selected from the group consisting of any naturally occurring amino acid, a suitable isostere, and corresponding D-amino acids;
Xaa⁴ is an amino acid, aliphatic acid, alicyclic acid, or modified 2-methyl aromatic acid having a side chain with one or two carbons, and capable of forming a thioether bond with Xaa¹⁰;
Xaa⁵ is selected from the group consisting of N(alpha)-Me-Arg, Arg, HomoArg, Dap, Dab, Arg-Me-sym, Arg-Me-asym, 4-Guan, Cit, Cav, N-Me-Lys, Phe (4-quanidino), Phe (4-carbomyl amino), Phe(4-NH₂), N-Me-Homo-Arg, Tyr and His, and suitable isostere replacements;
Xaa⁶ is selected from the group consisting of Ser, Gly, Thr, Ile, and suitable isostere replacements;
Xaa⁷ is selected from the group consisting of Asp, N-Me-Asp, Asp(OMe), D-Asp, and a suitable isostere replacement; wherein in certain embodiments, if Formula (I-2) is directed to a peptide dimer subunit then Xaa⁷ is selected from the group consisting of Asp, N-Me-Asp, D-Asp, and a suitable isostere replacement;
Xaa⁸ is selected from the group consisting of Thr, Gln, Ser, Asp, Pro, Gly, His, Ala, Ile, Phe, Lys, Arg, Asn, Glu, Val, Tyr, Trp, Leu, Met, hLeu, Nle and N-Methyl amino acids including N-Me-Thr;
Xaa⁹ is selected from the group consisting of Gln, Asn, Asp, Pro, Gly, Ala, Phe, Leu, Glu, Ile, Val, HomoLeu, n-Butyl Ala, n-Pentyl Ala, n-Hexyl Ala, Nle, cyclobutyl-Ala, N-Me-Leu, Cpa, Aoc and suitable isostere replacements; and
Xaa¹⁰ is selected from the group consisting of Cys, N-Me-Cys, D-Cys, HomoCys, Pen, D-Pen, modified HomoSer and modified Ser; wherein in certain embodiments, if Formula (I-2) is directed to a peptide dimer subunit, then Xaa¹⁰ is selected from the group consisting of Cys, N-Me-Cys, D-Cys, HomoCys, Pen, and D-Pen;
Xaa¹¹ is absent, or Xaa¹¹ is selected from the group consisting of or selected from the group consisting of: aromatic amino acids, substituted aromatic amino acids, Tic, and corresponding D-amino acids and suitable isostere replacements;
Xaa¹² is absent, or Xaa¹² is selected from the group consisting of: aromatic amino acids, substituted aromatic amino acids, Glu, D-Glu, homoGlu, Asp, D-Asp, D-homoGlu, Gla, beta-Homo-Glu, Tic, and corresponding D-amino acids and suitable isosteres;
Xaa¹³ is absent, or Xaa¹³ is selected from the group consisting of Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Om, N-Me-Dap, N-Me-Dab, N-Me-Lys, D-N-Me-Lys, D-Dap, D-Dab, COOH, CONH₂, suitable isosteres, and corresponding D-amino acids; and
wherein some embodiments, if Formula (I-2) is directed to a peptide monomer, then Xaa¹⁴ is any amino acid; and
in other embodiments, if Formula (I-2) is directed to a peptide dimer subunit, then Xaa¹⁴ is selected from the group consisting of: any amino acid with an amine side chain, Lys, D-Lys, N-Me-Lys, D-N-Me-Lys, Orn, N-Me-Orn, Dab, N-Me-Dab, Dap, N-Me-Dap, Homo-Lys, D-Dap, D-Dab, D-Orn, Gln, Pro, Gly, His, Ala, Ile, Phe, Lys, Arg, Leu, Val, Tyr, Trp, Met, Glu, Ser, Asn, Gla, Cys, HomoCys, Pen, COOH, CONH₂, suitable isosteres, corresponding D-amino acids, and corresponding N-Methyl amino acids.

The present invention also contemplates reverse order thioether bond embodiments of Formula (I-2), wherein Xaa¹⁰ is an amino acid, aliphatic acid, alicyclic acid, or modified 2-methylbenzoyl moiety acid having a free NH₂ group, and capable of forming a thioether bond with Xaa⁴; and Xaa⁴ is selected from the group consisting of Cys, N-Me-Cys, D-Cys, HomoCys, Pen, D-Pen; wherein in certain embodiments, Xaa⁴ is selected from the group consisting of Cys, N-Me-Cys, D-Cys, HomoCys, and Pen.

In one aspect, the present invention provides a peptide (e.g. a peptide monomer, a peptide dimer, or a peptide dimer subunit) comprising the structure according to Formula (I-3)(SEQ ID NO: 35):
Xaa¹-Xaa²-Xaa³-Xaa⁴-Xaa⁵-Xaa⁶-Xaa⁷-Xaa⁸-Xaa⁹-Xaa¹⁰-Xaa¹¹-Xaa¹²-Xaa¹³-Xaa¹⁴ Formula (I-3)), or a pharmaceutically acceptable salt thereof, wherein:
Xaa¹ is absent, Ac, or any amino acid;
Xaa² is absent, Ac, or any amino acid;
Xaa³ is absent, Ac, or any amino acid;
Xaa⁴ is selected from the group consisting of Cys, HomoCys, Pen, Homo-Ser-Cl, Homo-Ser, and a 2-methylbenzoyl moiety;
Xaa⁵ is selected from the group consisting of: N-Me-Arg, Arg, N-Me-Lys, Phe (4-quanidino), Phe(4-carbonylamino), Cit, Phe(4-NH₂), N-Me-Homo-Arg, Homo-Arg, Tyr and His;
Xaa⁶ is Ser, Gly, Ile or Thr; wherein in some embodiments, if Formula I-3 is directed to a peptide monomer then Xaa⁶ is Ser;
Xaa⁷ is Asp or D-Asp;
Xaa⁸ is selected from the group consisting of: Thr, Val, Ile, Leu, hLeu and Nle;
Xaa⁹ is selected from the group consisting of: Leu, Nle, Cpa, Cba, HomoLeu, Aoc, and N-Me-Leu;
Xaa¹⁰ is selected from the group consisting of: Cys, D-Cys, HomoCys, Pen, modified HomoSer and modified Ser; wherein in some embodiments, if Formula I-3 is directed to a peptide monomer, then Xaa¹⁰ is selected from the group consisting of: Cys, D-Cys, HomoCys, and Pen;
Xaa¹¹ is absent or selected from the group consisting of: aromatic amino acids, and substituted aromatic amino acids;
Xaa¹² is absent or selected from the group consisting of: aromatic amino acids, substituted aromatic amino acids, Glu, D-Glu, homoGlu, Asp, D-Asp, D-homoGlu, Gla, beta-Homo-Glu, and corresponding D-amino acids and suitable isosteres;
Xaa¹³ is absent or any amino acid, wherein in particular embodiments, Xaa¹³ is absent or Pro; and
wherein in some embodiments, if Formula I-3 is directed to a peptide monomer, then Xaa⁴ is any amino acid; and
wherein other embodiments, if Formula I-3 is directed to a peptide dimer subunit, then Xaa⁴ is absent or selected from the group consisting of: any amino acid with an amine side chain, Lys, D-Lys, N-Me-Lys, D-N-Me-Lys, Orn, N-Me-Orn, Dab, N-Me-Dab, Dap, N-Me-Dap, Homo-Lys, D-Dap, D-Dab, D-Orn, Gln, Pro, Gly, His, Ala, Ile, Phe, Lys, Arg, Leu, Val, Tyr, Trp, Met, Glu, Ser, Asn, Gla, Cys, HomoCys, Pen, COOH, CONH₂, suitable isosteres, corresponding D-amino acids, and corresponding N-Methyl amino acids.

The present invention also includes reverse orientation thioether bond embodiments of Formula (I-3), wherein Xaa¹⁰ is selected from the group consisting of Homo-Ser-Cl, Homo-Ser, modified Homo-Ser (e.g., Homo Ser(OTBDMS)) and a 2-methylbenzoyl moiety with free NH₂ group; and Xaa⁴ is selected from the group consisting of: Cys, D-Cys, HomoCys, Pen; wherein in some embodiments, Xaa¹⁰ is selected from the group consisting of: Homo-Ser, modified Homo-Ser and a 2-methylbenzoyl moiety.

In some embodiments of any of the peptides described herein, including but not limited to those of Formula (I), (V), (I-1), (I-2), and (I-3), Xaa⁴ is selected from Cys, HomoCys, Pen, and a 2-methylbenzoyl moiety. In certain embodiments, Xaa⁴ is selected from the group consisting of a modified Ser, a modified HomoSer, a suitable isostere, and corresponding D-amino acids. In one embodiment, Xaa⁴ is a Homo-Ser-Cl (before the thioether bond is formed with Xaa¹⁰ whereby the Cl is removed) or a HomoSer precursor (e.g., HomoSer(O-TBDMS). In other instances, Xaa⁴ is an aliphatic acid having from one to four carbons and forming a thioether bond with Xaa¹⁰. In some instances, Xaa⁴ is a five- or six-membered alicyclic acid having a modified 2-methyl group that forms a thioether bond with Xaa¹⁰. In some instances, Xaa⁴ is a 2-methylbenzoyl moiety. In some embodiments, the amino acid directly carboxyl to Xaa¹⁰ is an aromatic amino acid. In some embodiments, Xaa⁷ is Asp.

One of skill in the art will appreciate that certain amino acids and other chemical moieties are modified when bound to another molecule. For example, an amino acid side chain may be modified when it forms an intramolecular bridge with another amino acid side chain. In addition, when Homo-Ser-Cl binds to an amino acid such as Cys or Pen via a thioether bond, the Cl moiety is released. Accordingly, as used herein, reference to an amino acid or modified amino acid, such as Homo-Ser-Cl, present in a peptide dimer of the present invention (e.g., at position Xaa⁴ or position Xaa¹⁰) is meant to include the form of such amino acid or modified amino acid present in the peptide both before and after forming the intramolecular bond.

In some embodiments of any of the peptides described herein, including but not limited to those of Formula (I), (V), (I-1), (I-2), and (I-3), Xaa¹¹ is selected from the group consisting of: Gly, Gln, Asn, Asp, Ala, Ile, Leu, Val, Met, Thr, Lys, Trp, Tyr, His, Glu, Ser, Arg, Pro, Phe, Sar, 1-Nal, 2-Nal, D-1-Nal, D-2-Nal, HPhe, D-Phe, D-Tyr, Phe(4-F), O-Me-Tyr, dihydro-Trp, Dap, Dab, Dab(Ac), Orn, D-Orn, N-Me-Orn, N-Me-Dap, D-Dap, D-Dab, Bip, Ala(3,3diphenyl), Biphenyl-Ala, aromatic ring substituted Phe, aromatic ring substituted Trp, aromatic ring substituted His, hetero aromatic amino acids, N-Me-Lys, N-Me-Lys(Ac), 4-Me-Phe, and corresponding D-amino acids and suitable isostere replacements. In particular embodiments of any of the monomer peptides described herein, Xaa¹¹ is an aromatic amino acid or a substituted aromatic amino acid. In certain embodiments, Xaa¹¹ is Phe (4tBu), D-Lys, N-Me-Lys, or D-N-Me-Lys.

In some embodiments of any of the peptides described herein, including but not limited to those of Formula (I), (V), (I-1), (I-2), and (I-3), Xaa¹² is selected from the group consisting of Glu, Amide, Lys, COOH, Gln, Pro, Gly, His, Ala, Ile, Phe, Arg, Leu, Val, Tyr, Trp, Met, Gla, Ser, Asn, D-Glu, β-HGlu, 2-Nal, 1-Nal, D-Asp, Bip, β-HPhe, β-Glu, D-Tyr, D-Phe, D-Lys, Dap, Dab, Orn, D-Orn, N-Me-Orn, N-Me-Dap, N-Me-Dab, N-Me Lys, D-Dap, D-Dab, suitable isosteres, and corresponding D-amino acids.

In particular embodiments of of any of the compounds and genuses described herein, Xaa⁵ is selected from the group consisting of Cit, Phe(4-carbomyl amino), and N-Me-Homo-Arg; Xaa⁸ is selected from the group consisting of Leu, HomoLeu, Nle and Val; Xaa⁹ is selected from the group consisting of: Cba, HomoLeu, and Cpa; Xaa¹¹ is selected from the group consisting of Tic, Phe(2-carbomyl), Phe(3-carbomyl), Phe (4-COOH), Phe(4-OMe), and Phe(4tBu); Xaa¹² is selected from the group consisting of Aic, Gln, Cit, Glu(OMe), D-His, Tic, Phe(3-COOH), D-Arg, Bip, D-Trp, Phe, D-Phe, D-Val, D-Thr, D-1-Nal, D-2-Nal, Thr, Val; or Xaa¹³ is Pro.

In particular embodiments of any of the peptide described herein, including those of Formula (I), (V), (I-1), (I-2), and (I-3), Xaa⁸ is not Pro. In particular embodiments of any of the peptide described herein, including those of Formula (I), (V), (I-1), (I-2), and (I-3), Xaa⁹ is not Pro.

In certain embodiments of any of the peptides (e.g. peptide monomers, peptide dimers or peptide dimer subunits) described herein, including but not limited to those of Formula (I), (V), (I-1), (I-2), and (I-3), Xaa¹⁴ is selected from the group consisting of Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, N-Me-Lys, D-N-Me-Lys, D-Dap, D-Dab, COOH, CONH₂, suitable isosteres, corresponding D-amino acids, and corresponding N-Methyl amino acids. In certain embodiments, Xaa¹⁴ is D-Lys, N-Me-Lys, Dap, or Dab. In some embodiments of any of the peptide dimer subunits, Xaa¹⁴ (or the C-terminal amino acid) is Cys, HomoCys or Pen.

In some embodiments of any of the peptides (e.g. peptide monomers, peptide dimers or peptide dimer subunits) described herein, including but not limited to those of Formula (I), (V), (I-1), (I-2), and (I-3), Xaa¹⁴ is selected from the group consisting of any amino acid with an amine side chain, Gln, Pro, Gly, His, Ala, Ile, Phe, Lys, Arg, Leu, Val, Tyr, Trp, Met, Glu, Ser, Asn, Gla, Dap, Dab, Orn, D-Om, D-Lys, N-Me-Orn, N-Me-Dap, N-Me-Dab, N-Me-Lys, D-N-Me-Lys, D-Dap, D-Dab, COOH, CONH₂, suitable isosteres, corresponding D-amino acids, and corresponding N-Methyl amino acids

In some embodiments of any of the peptides described herein, including but not limited to those of Formula (I), (V), (I-1), (I-2), and (I-3), Xaa¹⁴ is selected from the group consisting of: any amino acid with a free amine side chain, Lys, D-Lys, N-Me-Lys, D-N-Me-Lys, Orn, Dab, Dap, Homo-Lys, D-Dap, D-Dab, or D-Orn.

In some embodiments of any of the peptides (e.g. peptide monomers, peptide dimers or peptide dimer subunits) described herein, including but not limited to those of Formula (I), (V), (I-1), (I-2), and (I-3), the amino acid residue directly C-terminal to Xaa¹⁰ is an aromatic amino acid. In certain embodiments, the amino acid directly C-terminal to Xaa¹⁰ is selected from aromatic amino acids, substituted aromatic amino acids, and Tic. In certain embodiments, the amino acid directly C-terminal to Xaa¹⁰ is an aromatic amino acid.

In one embodiment of Formula (I-1), herein referred to as Formula (I-A) (SEQ ID NO: 36);
Xaa¹ is absent or any amino acid;
Xaa² is absent or any amino acid;
Xaa³ is absent or any amino acid;
Xaa⁴ is a 2-methyl-benzoyl moiety or a modified HomoSer, optionally Homo-Ser-Cl;
Xaa⁵ is selected from the group consisting of N-Me-Arg, Arg, N-Me-Lys, Phe (4-quanidino), Phe(4-carbonylamino), Cit, Phe(4-NH₂), N-Me-Homo-Arg, Homo-Arg, Tyr and His;
Xaa⁶ is Ser, Gly, Thr or Ile; wherein in some embodiments, if Formula (I-A) is directed to a peptide dimer subunit, then Xaa⁶ is Ser;
Xaa⁷ is Asp or D-Asp;
Xaa⁸ is selected from the group consisting of: Thr, Val, Ile, Leu, hLeu, Nle, and Val;
Xaa⁹ is selected from the group consisting of: Leu, Nle, Cpa, Cba, HomoLeu, Aoc, and N-Me-Leu; wherein in some embodiments, if Formula I-A is directed to a monomer peptide, then Xaa⁹ is selected from the group consisting of: Leu, Nle, Cpa, HomoLeu, Aoc, and N-Me-Leu;
Xaa¹⁰ is Pen, Cys, D-Cys or HomoCys; and
Xaa¹¹ is absent or selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Phe (2-carbomyl), Tyr(Me), HomoPhe, N-Me-Phe, N-Me-Tyr, Ser, Sar, Dihydro Trp, Ile, Leu, Arg, Thr, Sar, and Ser; wherein in some embodiments, if Formula (I-A) is directed to a dimer peptide subunit, then Xaa¹¹ is absent or selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Phe (2-carbomyl), Tyr(Me), HomoPhe, N-Me-Phe, N-Me-Tyr, Ser, Sar, Dihydro Trp, Ile, Leu, Arg, and Thr; and
Xaa¹² is absent or selected from the group consisting of: any aromatic amino acid, Glu, D-Glu, homoGlu, Asp, D-Asp, D-homoGlu, D-Asp, Gla, beta-homo-Glu, corresponding D-amino acid, and isosteres; wherein in some embodiments, if Formula (I-A) is directed to a peptide monomer, then Xaa¹² is absent or selected from the group consisting of: any aromatic amino acid, Glu, D-Glu, homoGlu, Asp, D-Asp, D-homoGlu, Gla, beta-homo-Glu, corresponding D-amino acid, and isosteres;
wherein in some embodiments, if Formula (I-A) is directed to a peptide monomer, then Xaa¹³ is absent or any amino acid; and
wherein in other embodiments, if Formula (I-A) is directed to a peptide dimer subunit, then Xaa¹³ is absent;
wherein in some embodiments, if Formula (I-A) is directed to a peptide monomer, then Xaa¹⁴ is any amino acid; and
wherein other embodiments, if Formula (I-A) is directed to a peptide dimer subunit, then Xaa⁴ is selected from the group consisting of: any amino acid with a free amino group on a side chain, Lys, D-Lys, N-Me-Lys, D-N-Me-Lys, Om, Dab, Dap, Homo-Lys, D-Dap, D-Dab, Cys, HomoCys, Pen, or D-Orn.

In certain embodiments, Formula (I-A) is directed to a peptide monomer and Xaa¹³ is absent.

In one embodiment of Formula (I-1), herein referred to as Formula (I-B) (SEQ ID NO: 37),
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser, Gly, Thr, or Ile; wherein in some embodiments, if Formula (I-B) is directed to a peptide dimer subunit then Xaa⁶ is Ser;
Xaa⁷ is Asp or D-Asp;
Xaa⁸ is selected from the group consisting of: Thr, Val, Ile, Leu, hLeu and Nle;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Ser and any substituted aromatic amino acid and corresponding D-amino acids; wherein in some embodiments, if Formula (I-B) is directed to a peptide dimer subunit, then Xaa¹¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Dihydro Trp, Ile, Leu, Ser, Arg, Thr, Sar, and Ser;
Xaa¹² is selected from the group consisting of: any aromatic amino acid, Glu, D-Glu, homoGlu, Asp, D-Asp, D-homoGlu, Gla, beta-homo-Glu, corresponding D-amino acid and isosteres;
Xaa¹³ is absent;
wherein some embodiments, if Formula (I-B) is directed to a peptide monomer, then Xaa¹⁴ is any amino acid; and

in other embodiments, if Formula (I-B) is directed to a peptide dimer subunit, then Xaa⁴ is selected from the group consisting of: Lys, D-Lys, N-Me-Lys, D-N-Me-Lys, Orn, Dab, Dap, Homo-Lys, D-Dap, D-Dab, Cys, HomoCys, Pen, or D-Orn.

In one embodiment of Formula (I-1), herein referred to as Formula (I-C) (SEQ ID NO: 38),
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser, Gly, Thr, or Ile; wherein in some embodiments, if Formula (I-C) is directed to a peptide dimer subunit, then Xaa⁶ is Ser;
Xaa⁷ is Asp or D-Asp;
Xaa⁸ is selected from the group consisting of: Thr, Val, Ile, Leu, hLeu and Nle;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3). Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Sar, Dihydro Trp, Ile, Leu, Ser, Arg, Thr, Sar, and Ser; or
Xaa¹² is selected from the group consisting of: any aromatic amino acid, Glu, D-Glu, homoGlu, Asp, D-Asp, D-homoGlu, Gla, beta-homo-Glu, corresponding D-amino acid and isosteres;
Xaa¹³ is absent or any amino acid; wherein in other embodiments, if Formula (I-C) is directed to a peptide dimer subunit, then Xaa¹³ is absent; and
wherein in some embodiments, if Formula (I-C) is directed to a peptide monomer subunit then Xaa¹⁴ is any amino acid; and
wherein in other embodiments, if Formula (I-C) is directed to a peptide dimer subunit then Xaa¹⁴ is selected from the group consisting of: Lys, D-Lys, N-Me-Lys, D-N-Me-Lys, Orn, Dab, Dap, Homo-Lys, D-Dap, D-Dab, Cys, HomoCys, Pen, or D-Orn.

In certain embodiments, Formula (I-C) is directed to a peptide monomer and Xaa¹³ is absent.

In one embodiment of Formula (I-1), herein referred to as Formula (I-D) (SEQ ID NO: 39),
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, and Ser;
Xaa¹² is absent or selected from the group consisting of: any aromatic amino acid, Glu, D-Glu, homoGlu, Asp, D-Asp, D-homoGlu, Gla, beta-homo-Glu, corresponding D-amino acid and isosteres;
Xaa¹³ is absent; and
wherein in some embodiments, if Formula (I-D) is directed to a peptide monomer then Xaa¹⁴ is any amino acid; and wherein in other embodiments, if Formula (I-D) is directed to a peptide dimer subunit, then Xaa¹⁴ is selected from the group consisting of: Lys, D-Lys, N-Me-Lys, D-N-Me-Lys, Orn, Dab, Dap, Homo-Lys, D-Dap, D-Dab, Cys, HomoCys, Pen, or D-Orn.

In one embodiment of Formula (I-1), herein referred to as Formula (I-E) (SEQ ID NO: 40),
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3). Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, and Ser;
Xaa¹² is absent or selected from the group consisting of: any aromatic amino acid, Glu, D-Glu, and beta-homo-Glu;
Xaa¹³ is absent; and,
wherein in some embodiments, if Formula (I-E) is directed to a peptide monomer, then Xaa¹⁴ is any amino acid; and in other embodiments, if Formula (I-E) is directed to a peptide dimer subunit, then Xaa⁴ is selected from the group consisting of: Lys, D-Lys, N-Me-Lys, D-N-Me-Lys, Orn, Dab, Dap, Homo-Lys, D-Dap, D-Dab, Cys, HomoCys, Pen, or D-Orn.

In one embodiment of Formula (I-1), herein referred to as Formula (I-F) (SEQ ID NO: 41),
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3). Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, and Ser;
Xaa¹² is selected from the group consisting of: any aromatic amino acid, Glu, D-Glu, beta-homo-Glu, corresponding D-amino acid and isosteres;
Xaa¹³ is absent; and
wherein in some embodiments, if Formula (I-F) is directed to a peptide monomer, then Xaa⁴ is any amino acid; and wherein in some embodiments, if Formula (I-F) is directed to a peptide dimer subunit, then Xaa¹⁴ is selected from the group consisting of: Lys, D-Lys, N-Me-Lys, D-N-Me-Lys, Orn, Dab, Dap, Homo-Lys, D-Dap, D-Dab, Cys, HomoCys, Pen, or D-Orn.

In certain embodiments, Xaa⁴ is selected from the group consisting of: Lys, D-Lys, N-Me-Lys, D-N-Me-Lys.

In one embodiment of Formula (I-1), herein referred to as Formula (I-G) (SEQ ID NO: 42),
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3). Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, and Ser;
Xaa¹² is selected from the group consisting of: any aromatic amino acid, Glu, D-Glu, and beta-homo-Glu;
Xaa¹³ is absent; and
wherein in some embodiments, if Formula I-G is directed to a peptide monomer, then Xaa¹¹ is any amino acid; and wherein in other embodiments, if Formula I-G is directed to a peptide dimer subunit, then Xaa¹¹ is selected from the group consisting of: Lys, D-Lys, N-Me-Lys, D-N-Me-Lys, Orn, Dab, Dap, Homo-Lys, D-Dap, D-Dab, Cys, HomoCys, Pen, or D-Orn.

In certain embodiments, Xaa¹¹ is selected from the group consisting of: D-Lys, N-Me-Lys, and D-N-Me-Lys.

In one embodiment of Formula (I-1), herein referred to as Formula (I-H) (SEQ ID NO: 43),
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp;
Xaa⁸ is Thr or Val;
Xaa⁹ is Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3). Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, and Ser;
Xaa¹² is selected from the group consisting of: any aromatic amino acid, Glu, D-Glu, and beta-homo-Glu;
Xaa¹³ is absent; and
wherein in some embodiments, if Formula I-H is directed to a peptide monomer, then Xaa⁴ is any amino acid; and wherein in some embodiments, if Formula I-H is directed to a peptide dimer subunit, then Xaa⁴ is selected from the group consisting of: D-Lys, N-Me-Lys, and D-N-Me-Lys.

In one embodiment of Formula (I-1), herein referred to as Formula (I-I) (SEQ ID NO: 44),
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3). Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), and HomoPhe;
Xaa¹² is selected from the group consisting of: any aromatic amino acid, Glu, D-Glu, and beta-homo-Glu;
Xaa¹³ is absent; and
wherein in some embodiments, if Formula I-I is directed to a peptide monomer then Xaa¹⁴ is any amino acid; and wherein in other embodiments, if Formula I-I is directed to a peptide dimer subunit, then Xaa⁴ is selected from the group consisting of: D-Lys, N-Me-Lys, and D-N-Me-Lys.

In certain embodiments of Formulas (I), (V), (I-1), (I-2), (I-3), (V), or any of (I-A), (I-B), I-C), (I-D), (I-E), (I-F), (I-G), (I-H), and (I-I), Xaa¹¹ may also be Bpa, Phe(3-Me), Phe(2-Me), Phe(2-CF3), or P-Me-Phe.

In certain embodiments of Formulas (I), (V), (I-1), (I-2), (I-3), (V) or any of (I-A), (I-B), I-C), (I-D), (I-E), (I-F), (I-G), (I-H), and (I-I), Xaa¹² may also be N-Me-Glu, N-Me-Asp, or alpha-H-Glu.

In particular embodiments of Formulas (I), (V), (I-1), (I-2), (I-3), (V), or any of (I-A), (I-B), I-C), (I-D), (I-E), (I-F), (I-G), (I-H), and (I-I), e.g., when the peptide is a dimer, Xaa¹⁴ is selected from the group consisting of: Lys, D-Lys, N-Me-Lys, D-N-Me-Lys, Orn, Dab, Dap, Homo-Lys, D-Dap, D-Dab, Cys, HomoCys, Pen, or D-Om, while in other embodiments, Xaa¹⁴ is selected from D-Lys, N-Me-Lys, and D-N-Me-Lys.

In one embodiment of Formula (I-1), Xaa¹ is absent, or Xaa¹ is any amino acid;
Xaa² is absent, or Xaa² is any amino acid;
Xaa³ is absent, or Xaa³ is any amino acid;
Xaa⁴ is an amino acid, aliphatic acid, alicyclic acid, or modified 2-methyl aromatic acid having a side chain with one or two carbons, and capable of forming a thioether bond with Xaa¹⁰;
Xaa⁵ is selected from the group consisting of N(alpha)-Me-Arg, Arg, HomoArg, Dap, Dab, Arg-Me-sym, Arg-Me-asym, 4-Guan, Cit, Cav, N-Me-Lys, Phe(4-quanidino), Phe(4-carbamoyl amino), Phe(4-NH₂), N-Me-HomoArg, Tyr, His, and suitable isostere replacements;
Xaa⁶ is selected from the group consisting of Ser, Gly, Thr, Ile, and suitable isostere replacements;
Xaa⁷ is selected from the group consisting of Asp, N-Me-Asp, Asp(OMe), D-Asp, and suitable isostere replacements;
Xaa⁸ is selected from the group consisting of Thr, Gln, Ser, Asp, Pro, Gly, His, Ala, Ile, Phe, Lys, Arg, Asn, Glu, Val, Tyr, Trp, Leu, Met, HomoLeu, Nle, and N-Methyl amino acids including N-Me-Thr;
Xaa⁹ is selected from the group consisting of Gln, Asn, Asp, Pro, Gly, Ala, Phe, Leu, Glu, Ile, Val, HLeu, n-Butyl Ala, n-Pentyl Ala, n-Hexyl Ala, Nle, cyclobutyl-Ala, Cpa, Aoc, N-Me-Leu, and suitable isostere replacements;
Xaa¹⁰ is selected from the group consisting of Cys, N-Me-Cys, D-Cys, HCys, Pen, D-Pen, and Pen(═O);
Xaa¹¹ is absent or is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Phe (2-carbomyl), Tyr(Me), HomoPhe, N-Me-Phe, N-Me-Tyr, Ser, Sar, Dihydro Trp, Ile, Leu, Ser, Arg, Thr, Sar, and Ser, aromatic amino acids, substituted aromatic amino acids, Gly, Gln, Asn, Asp, Ala, Ile, Leu, Val, Met, Thr, Lys, Trp, Tyr, His, Glu, Ser, Arg, Pro, Phe, Sar, 1-Nal, 2-Nal, D-1-Nal, D-2-Nal, HPhe, D-Phe, D-Tyr, Phe(4-F), O-Me-Tyr, dihydro-Trp, Dap, Dab, Dab(Ac), Orn, D-Orn, N-Me-Orn, N-Me-Dap, D-Dap, D-Dab, Bip, Ala(3,3diphenyl), Biphenyl-Ala, aromatic ring substituted Phe, aromatic ring substituted Trp, aromatic ring substituted His, hetero aromatic amino acids, N-Me-Lys, N-Me-Lys(Ac), 4-Me-Phe, Phe(4tBu), Phe(4-OMe), Phe(4-COOH), Phe(2-carbomyl), Phe(3-carbomyl), Phe(CF3), Phe(2,4-diCl), Phe(3,4-diCl), Aic, N-Me-Tyr, N-Me-Phe, Tic, Phe(4CF3), Bpa, Phe(3-Me), Phe(2-Me), Phe(2-CF3), P-Me-Phe, and corresponding D-amino acids and suitable isostere replacements;
Xaa¹² is absent or selected from the group consisting of aromatic amino acids, substituted aromatic amino acids, Glu, D-Glu, HomoGlu, Beta-Homo-Glu, Asp, D-HomoGlu, Amide, Lys, COOH, CONH₂, Gln, Pro, Gly, His, Ala, Ile, Phe, Arg, Leu, Val, Tyr, Trp, Met, Gla, Ser, Asn, D-Glu, β-HGlu, 2-Nal, 1-Nal, D-Asp, Bip, β-HPhe, β-Glu, D-Tyr, D-Phe, D-Lys, Dap, Dab, Orn, D-Om, N-Me-Orn, N-Me-Dap, N-Me-Dab, N-Me Lys, D-Dap, D-Dab, D-His, F(4-COOH), Tic, D-Trp, D-Leu, D-Arg, D-Thr, N-Me-Glu, N-Me-Asp, alpha-H-Glu, suitable isosteres, and corresponding D-amino acids;
Xaa¹³ is absent or any amino acid; and
Xaa¹¹ is absent or any amino acid.

In other embodiments, Xaa¹ is absent or any amino acid;
Xaa² is absent or any amino acid;
Xaa³ is absent or any amino acid;
Xaa⁴ is a 2-methyl-benzoyl moiety or a modified HomoSer, optionally Homo-Ser-Cl;
Xaa⁵ is selected from the group consisting of N-Me-Arg, Arg, N-Me-Lys, Phe (4-quanidino), Phe(4-carbonylamino), Cit, Phe(4-NH₂), N-Me-Homo-Arg, Homo-Arg, Tyr and His;
Xaa⁶ is Ser, Gly, Thr or Ile;
Xaa⁷ is Asp or D-Asp;
Xaa⁸ is selected from the group consisting of: Thr, Val, Ile, Leu, hLeu, Nle, and Val;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys; and
Xaa¹¹ is absent or selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Phe (2-carbomyl), Tyr(Me), HomoPhe, N-Me-Phe, N-Me-Tyr, Ser, Sar, Dihydro Trp, Ile, Leu, Arg, Thr, Sar, Bpa, Phe(3-Me), Phe(2-Me), Phe(2-CF3), P-Me-Phe, and Ser;
Xaa¹² is absent or selected from the group consisting of: any aromatic amino acid, Glu, D-Glu, homoGlu, Asp, D-Asp, D-homoGlu, D-Asp, Gla, beta-homo-Glu, N-Me-Glu, N-Me-Asp, alpha-H-Glu, corresponding D-amino acid, and isosteres;
Xaa¹³ is absent or any amino acid; and
Xaa¹⁴ is any amino acid.

In other embodiments,
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser, Gly, Thr, or Ile;
Xaa⁷ is Asp or D-Asp;
Xaa⁸ is selected from the group consisting of: Thr, Val, Ile, Leu, hLeu and Nle;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys;
Xaa¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Bpa, Phe(3-Me), Phe(2-Me), Phe(2-CF3), 3-Me-Phe, Ser and any substituted aromatic amino acid and corresponding D-amino acids;
Xaa¹² is selected from the group consisting of: any aromatic amino acid, Glu, D-Glu, homoGlu, Asp, D-Asp, D-homoGlu, Gla, beta-homo-Glu, N-Me-Glu, N-Me-Asp, alpha-H-Glu, corresponding D-amino acid and isosteres;
Xaa¹³ is absent; and
Xaa¹⁴ is any amino acid.

In other embodiments,
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser, Gly, Thr, or Ile;
Xaa⁷ is Asp or D-Asp;
Xaa⁸ is selected from the group consisting of: Thr, Val, Ile, Leu, hLeu and Nle;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3). Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Sar, Dihydro Trp, Ile, Leu, Ser, Arg, Thr, Sar, Bpa, Phe(3-Me), Phe(2-Me), Phe(2-CF3), P-Me-Phe, and Ser;
Xaa¹² is selected from the group consisting of: any aromatic amino acid, Glu, D-Glu, homoGlu, Asp, D-Asp, D-homoGlu, Gla, beta-homo-Glu, N-Me-Glu, N-Me-Asp, alpha-H-Glu, corresponding D-amino acid and isosteres;
Xaa¹³ is absent or any amino acid; and
Xaa¹⁴ is any amino acid.

In other embodiments:
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Bpa, Phe(3-Me), Phe(2-Me), Phe(2-CF3), 3-Me-Phe, and Ser;
Xaa¹² is absent or selected from the group consisting of: any aromatic amino acid, Glu, D-Glu, homoGlu, Asp, D-Asp, D-homoGlu, Gla, beta-homo-Glu, N-Me-Glu, N-Me-Asp, alpha-H-Glu, corresponding D-amino acid and isosteres;
Xaa¹³ is absent; and
Xaa¹⁴ is any amino acid.

In other embodiments:
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3). Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Bpa, Phe(3-Me), Phe(2-Me), Phe(2-CF3), 3-Me-Phe, and Ser;
Xaa¹² is absent or selected from the group consisting of: any aromatic amino acid, Glu, D-Glu, beta-homo-Glu, N-Me-Glu, N-Me-Asp, alpha-H-Glu;
Xaa¹³ is absent; and
Xaa¹⁴ is any amino acid.

In other embodiments:
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3). Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Bpa, Phe(3-Me), Phe(2-Me), Phe(2-CF3), 3-Me-Phe, and Ser;
Xaa¹² is selected from the group consisting of: any aromatic amino acid, Glu, D-Glu, beta-homo-Glu, N-Me-Glu, N-Me-Asp, alpha-H-Glu, corresponding D-amino acid and isosteres;
Xaa¹³ is absent; and
Xaa¹⁴ is any amino acid.

In other embodiments:
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3). Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Bpa, Phe(3-Me), Phe(2-Me), Phe(2-CF3), 3-Me-Phe, and Ser;
Xaa¹² is selected from the group consisting of: any aromatic amino acid, Glu, D-Glu, N-Me-Glu, N-Me-Asp, alpha-H-Glu, and beta-homo-Glu;
Xaa¹³ is absent; and
Xaa¹⁴ is any amino acid.

In other embodiments:
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp;
Xaa⁸ is Thr or Val;
Xaa⁹ is Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3). Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Bpa, Phe(3-Me), Phe(2-Me), Phe(2-CF3), 3-Me-Phe, and Ser;
Xaa¹² is selected from the group consisting of: any aromatic amino acid, Glu, D-Glu, N-Me-Glu, N-Me-Asp, alpha-H-Glu, and beta-homo-Glu;
Xaa¹³ is absent; and
Xaa¹⁴ is any amino acid.

In other embodiments:
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3). Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Tyr(Me), Bpa, Phe(3-Me), Phe(2-Me), Phe(2-CF3), P-Me-Phe, and HomoPhe;
Xaa¹² is selected from the group consisting of: any aromatic amino acid, Glu, D-Glu, N-Me-Glu, N-Me-Asp, alpha-H-Glu, and beta-homo-Glu;
Xaa¹³ is absent; and
Xaa¹⁴ is any amino acid.

In some embodiments of any of the peptides (e.g. peptide monomers, or peptide dimers or subunits thereof) described herein, including but not limited to those of Formula (I) (including (I-A)-(I-I), (I-1), (I-2) and (I-3)) or Formula (V), Xaa⁷ is Asp.

In some embodiments of any of the peptides (e.g. peptide monomers, or peptide dimers or subunits thereof) described herein, including but not limited to those of Formula (I) (including (I-A)-(I-I), (I-1), (I-2) and (I-3) or Formula (V)), the N-terminus of the peptide is acylated.

In some embodiments of any of the peptides (e.g. peptide monomers or peptide dimers or subunits thereof) described herein, including but not limited to those of Formula (I) (including (I-A)-(I-I), (I-1), (I-2) and (I-3) or Formula (V)), Xaa¹⁴ or the C-terminal amino acid does not comprise a free amine.

In some embodiments of any of the peptides (e.g. peptide monomers or peptide dimers or subunits thereof) described herein, including but not limited to those of Formula (I) (including (I-A)-(I-I), (I-1), (I-2) and (I-3) or Formula (V)), Xaa¹⁴ or the C-terminus comprises an NH₂ or an OH. In particular embodiments, Xaa¹³ is D-Lys Xaa¹⁴ or the C-terminus is an OH.

In some embodiments of any of the peptide (e.g. peptide monomers or peptide dimers or subunits thereof) described herein, including but not limited to those of Formula (I) (including (I-A)-(I-I), (I-1), (I-2) and (I-3) or Formula (V)), a free amine in the C-terminal amino acid of the peptide monomer is capped, e.g., with an acetyl group.

In some embodiments of any of the peptides (e.g. peptide monomers or peptide dimers or subunits thereof) described herein, including but not limited to those of Formula (I) (including (I-A), (I-I), (I-1), (I-2) and (I-3)) or Formula (V), the peptide monomer or dimer subunit comprises an intramolecular thioether bond between Xaa⁴ and Xaa¹⁰. In certain embodiments, Xaa⁴ is a 2-methylbenzoyl moiety, and Xaa¹⁰ is Pen. In certain embodiments, Xaa⁴ is Homo-Ser-Cl, and Xaa¹⁰ is Cys, D-Cys, or HomoCys.

In some embodiments of any of the peptides (e.g. peptide monomers or peptide dimers or subunits thereof) described herein, including but not limited to those of Formula (I) (including (I-A)-(I-I), (I-1), (I-2) and (I-3)) or Formula (V), at least one of Xaa¹, Xaa², Xaa³, Xaa⁵, Xaa⁷, Xaa⁸, Xaa⁹, Xaa¹¹, Xaa¹², Xaa¹³ and Xaa¹⁴ is N(alpha)Methylated.

In some instances of any of the peptides (e.g. peptide monomers or peptide dimers or subunits thereof) described herein, any of Xaa¹-Xaa⁴, and Xaa¹¹-Xaa¹⁴ are acylated. For example, in some instances one or more residues at positions Xaa¹-Xaa⁴, and Xaa¹¹-Xaa¹⁴ are acylated with an acylating organic compound selected from the group consisting of 2-me-Trifluorobutyl, Trifluoropentyl, Acetyl, Octonyl, Butyl, Pentyl, Hexyl, Palmityl, Lauryl, Oleoyl, and Lauryl, Trifluoromethyl butyric, cyclopentane carboxylic, cyclopropylacetic, 4-fluorobenzoic, 4-fluorophenyl acetic, 3-Phenylpropionic, tetrahedro-2H-pyran-4carboxylic, succinic acid, and glutaric acid. In some instances, small PEG (e.g., PEG4-PEG13) is used as spacer before acylations.

In certain embodiments, the N-terminus of a peptide monomer or peptide dimer subunit represented by Formula (I) (including (I-A)-(I-I), (I-1), (I-2) and (I-3)), or Formula (II) or Formula (V) or Formula (VI), or any other peptide described herein, can be modified by one to three suitable groups, as represented by Xaa¹, Xaa², and Xaa³ in Formula (I), (I-A), (I-B) and (I-C) or Formula (V). The N-terminus may further be acylated e.g., as described herein with respect to peptide monomers or peptide dimer subunits of Formula (I), Formula (V), Formula (II), and Formula (VI). In some instances, the N-terminus further comprises a suitable linker moiety or other modifying group.

Similarly, in certain embodiments, the C-terminus of a peptide monomer or dimer subunit represented by Formula (I) (including (I-A)-(I-I)), (I-1), (I-2) and (I-3), or Formula (V), or a peptide monomer or peptide dimer subunit of Formula (II), or any other peptide described herein, can be modified by a suitable group. For example, the C-terminus may be acylated. In some instances, the C-terminus further comprises a suitable linker moiety or modifying group, as disclosed herein. In certain embodiments, the C-terminus comprises NH₂ or OH.

In some embodiments, Xaa¹, Xaa², and Xaa³ of Formula (I) (including (I-1)-(I-I)), (I-1), (I-2) and (I-3) or Formula (V) are absent. In particular embodiments Xaa¹, Xaa², and Xaa³ of any peptide dimer subunit described herein are absent. In other embodiments, Xaa¹ is absent, and Xaa² and Xaa³ represent suitable groups for modifying the N-terminus of the peptide monomer or peptide dimer subunit. Further, in some embodiments Xaa¹ and Xaa² are absent, and Xaa³ represents a single suitable group for modifying the N-terminus of the peptide monomer or peptide dimer subunit.

With continued reference to the peptide monomers and peptide of the general formula of Formula (I), (I-1), (I-2) and (I-3) or Formula (V), Xaa¹⁻³ may comprise any naturally occurring amino acid, a suitable isostere, or corresponding D-amino acid. In some embodiments, at least one of Xaa¹⁻³ is absent. For example, in some instances Xaa¹ is absent, whereby Xaa² is the N-terminus. In other instances Xaa¹ and Xaa² are absent, whereby Xaa³ is the N-terminus. Further still, in some instances Xaa¹⁻³ are absent, whereby Xaa⁴ is the N-terminus. In some embodiments, the N-terminal residue is acylated or comprises a free amine. In some embodiments, the N-terminal residue of the peptide monomer or peptide dimer subunit is a 2-methyl benzoyl moiety (abbreviated herein as 2-benzyl).

In certain embodiments, peptide monomers, or peptide dimers having subunits of Formula (I) (including (I-A)-(I-I)), (I-1), (I-2) and (I-3) or Formula (V), or any other peptide described herein, the amino acid residue directly C-terminal to Xaa¹⁰ is an aromatic amino acid.

In other embodiments, the N-terminal residue of peptide monomers or peptide dimer subunits of Formula (I) (including (I-A)-(I-I), (I-1), (I-2) and (I-3)), or any other peptide described herein, further comprises a suitable linker moiety, e.g., a linker moiety, or modifying group selected from the group consisting of DIG, PEG4, PEG13, PEG25, PEG1K, PEG2K, PEG4K, PEG5K, Polyethylene glycol having molecular weight from 400 Da to 40,000 Da, PEG having a molecular weight of 40,000 Da to 80,000 Da, IDA, Ac-IDA, ADA, Glutaric acid, AADA, suitable aliphatic acids, suitable aromatic acids, and heteroaromatic acids.

In various embodiments of any of the peptides (e.g. peptide monomers, peptide dimers, or subunits thereof) described herein, one or more of the amino acids represented by Xaa¹⁻³ may be either absent or selected from the group consisting of any naturally occurring amino acid, a suitable isostere, and corresponding D-amino acids. When Xaa¹ and Xaa² are absent, Xaa³ is the N-terminus. When Xaa¹⁻³ are absent, Xaa⁴ is the N-terminus.

In some embodiments, Xaa⁴ is an amino acid residue having a side chain with one or two carbons, and forming a thioether bond with Xaa¹⁰. In some instances, Xaa⁴ is selected from the group consisting of modified Ser, modified HSer, a suitable isostere, and corresponding D-amino acids. In other instances, Xaa⁴ is an aliphatic acid having from one to four carbons and forming a thioether bond with Xaa¹⁰. In some instances, Xaa⁴ is a five- or six-membered alicyclic acid having a modified 2-methyl group that forms a thioether bond with Xaa¹⁰. In some embodiments, Xaa⁴ is a 2-methyl-benzoyl moiety or a modified form thereof. In certain embodiments, Xaa⁴ Cys, Pen, homocys, D-Pen, D-Cys or D-homocys. In certain embodiments, Xaa⁴ is 2-chloromethylbenzoic acid, 2-chloro-acetic acid, 3-choro-propanoic acid, 4-chloro-butyric acid, 3-chloro-isobutyric acid, Ser(Cl); Xaa¹⁰ is Cys, Pen, D-Cys, HomoCys; and the intramolecular bond is a thioether bond. One of skill in the art will appreciate that upon bonding with another amino acid, e.g., Xaa¹⁰, the Cl of hSer(Cl) will be removed.

For each embodiment of the peptide monomers or peptide dimer subunits of Formula (I) and (I-A) or Formula (V), and any of the peptide monomers or peptide dimers described herein, a thioether bond exists between Xaa⁴ and Xaa¹⁰ in the monomer peptides or in one or both of the peptide dimer subunits. Thus, the thioether peptide monomers or peptide dimer subunits of the present invention are cyclized through a thioether bond.

In some embodiments of any of the peptides described herein, Xaa⁵ is selected from the group consisting of N(alpha)-Me-Arg, Arg, HArg, Dap, Dab, Arg-Me-sym, Arg-Me-asym, 4-Guan, Cit, Cav, and suitable isostere replacements. In some embodiments, Xaa⁵ is N(alpha)Methylated. Preferably, Xaa⁵ is N-Me-Arg. In other embodiments, preferably Xaa⁵ is Arg.

In some embodiments of any of the peptides (e.g. peptide monomers, peptide dimers, or subunits thereof), described herein, Xaa⁶ is selected from the group consisting of Ser, Gly, Thr, Ile, and suitable isostere replacements. Preferably, Xaa⁶ is Ser. In some embodiments of any of the peptide dimer subunits described herein, Xaa⁶ is selected from the group consisting of Ser, Gly, Thr, Ile, and suitable isostere replacements. In some embodiments of any of the peptide monomers described herein, Xaa⁶ is selected from the group consisting of Ser, Gly, and suitable isostere replacements.

In some embodiments of any of the peptide monomers or dimers described herein, Xaa⁷ is selected from the group consisting of Asp, N-Me-Asp, D-Asp, Asp(OMe), and a suitable isostere replacements. In some embodiments of any of the peptide dimers described herein, Xaa⁷ is selected from the group consisting of Asp, N-Me-Asp, D-Asp, and a suitable isostere replacements. In some embodiments, Xaa⁷ is N(alpha)Methylated. Preferably, Xaa⁷ is Asp.

In some embodiments of any of the peptides described herein, Xaa⁸ is selected from the group consisting of Thr, Gln, Ser, Asp, Pro, Gly, His, Ala, Ile, Phe, Lys, Arg, Asn, Glu, Val, Tyr, Trp, Leu, Met, and N-Methyl amino acids including N-Me-Thr, and suitable isostere replacements. In some embodiments, Xaa⁸ is N(alpha)Methylated. Preferably, Xaa⁸ is Thr.

In some embodiments of any of the peptides described herein, Xaa⁹ is selected from the group consisting of Gln, Asn, Asp, Pro, Gly, Ala, Phe, Leu, Glu, Ile, Val, HLeu, n-Butyl Ala, n-Pentyl Ala, n-Hexyl Ala, Nle, cyclobutyl-Ala, N-Me-Leu, and suitable isostere replacements. In some embodiments, Xaa⁹ is N(alpha)Methylated. In certain embodiments, Xaa⁹ is Leu.

In some embodiments of any of the peptide monomers or peptide dimer subunits described herein, Xaa¹⁰ is selected from the group consisting of Cys, N-Me-Cys, D-Cys, HCys, Pen, and D-Pen. In some embodiments, Xaa¹⁰ is selected from the group consisting of Cys, N-Me-Cys, D-Cys, HCys, and Pen. In one embodiment, Xaa¹⁰ is Pen. In another embodiment, Xaa¹⁰ is preferably Cys.

In some embodiments of any of the peptides described herein, Xaa¹¹ is absent, or Xaa¹¹ is selected from the group consisting of Gly, Gln, Asn, Asp, Ala, Ile, Leu, Val, Met, Thr, Lys, Trp, Tyr, His, Glu, Ser, Arg, Pro, Phe, Sar, 1-Nal, 2-Nal, D-1-Nal, D-2-Nal, HPhe, Phe(4-F), O-Me-Tyr, dihydro-Trp, D-Phe, D-Tyr, Dap, Dab, Dab(Ac), Orn, D-Orn, N-Me-Orn, N-Me-Dap, D-Dap, D-Dab, Bip, Ala(3,3diphenyl), Biphenyl-Ala, aromatic ring substituted Phe, aromatic ring substituted Trp, aromatic ring substituted His, hetero aromatic amino acids, N-Me-Lys, N-Me-Lys(Ac), 4-Me-Phe, and corresponding D-amino acids and suitable isostere replacements. In some embodiments, Xaa¹¹ is preferably Trp. In some other embodiments, Xaa¹¹ is Phe. In some embodiments, Xaa¹¹ is F(4tBu), F(4-COOH), Bip, 1-Nal or 2-Nal. In particular embodiments of peptide monomers described herein, Xaa¹¹ is N(alpha)Methylated. In certain embodiments of peptide monomers or peptide dimer subunits described herein, Xaa¹¹ is Phe. In some embodiments, Xaa¹¹ is N(alpha)Methylated. Further, in some embodiments Xaa¹¹ is acylated.

In at least one embodiment of peptide monomers or peptide dimer subunits described herein, Xaa¹¹ is absent and Xaa¹⁰ is the C-terminus. When Xaa¹²⁻¹⁴ are absent, Xaa¹¹ is the C-terminus of the subunit. When Xaa¹¹ is the C-terminus of the subunit, Xaa¹¹ may be modified to include a suitable linker moiety in accordance with the present invention.

In some embodiments of peptide monomers or peptide dimers described herein, Xaa¹² is absent, or Xaa¹² is selected from the group consisting of Glu, Lys, COOH, CONH₂, Gln, Pro, Gly, His, Ala, Ile, Phe, Lys, Arg, Leu, Val, Tyr, Trp, Met, Gla, Ser, Asn, D-Glu, β-HGlu, 2-Nal, 1-Nal, D-Asp, Bip, β-HPhe, β-Glu, D-Tyr, D-Phe, D-Lys, Dap, Dab, Orn, D-Orn, N-Me-Orn, N-Me-Dap, N-Me-Dab, N-Me Lys, D-Dap, D-Dab, suitable isosteres, and corresponding D-amino acids. In some embodiments of peptide dimers described herein, Xaa¹² is absent, or Xaa¹² is selected from the group consisting of Glu, Lys, Gln, Pro, Gly, His, Ala, Ile, Phe, Lys, Arg, Leu, Val, Tyr, Trp, Met, Gla, Ser, Asn, D-Glu, β-HGlu, 2-Nal, 1-Nal, D-Asp, Bip, β-HPhe, β-Glu, D-Tyr, D-Phe, D-Lys, Dap, Dab, Orn, D-Orn, N-Me-Orn, N-Me-Dap, N-Me-Dab, N-Me Lys, D-Dap, D-Dab, suitable isosteres, and corresponding D-amino acids. In certain embodiments, Xaa¹² is Glu, D-Glu, β-HGlu, or Asp. In some embodiments, Xaa¹² is β-Hglu.

In some embodiments of the peptide monomer or peptide dimers described herein, Xaa¹³ and Xaa¹¹ are absent, and Xaa¹² is the C-terminus of the subunit. In some embodiments of the peptide dimers described herein, when Xaa¹² is the C-terminus of the subunit, Xaa¹² may be modified to include a suitable linker moiety in accordance with the present invention.

In some embodiments of any of the peptides (e.g. peptide monomers, peptide dimers, or subunits thereof) described herein, Xaa¹³ is absent, or Xaa¹³ is selected from the group consisting of Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, N-Me-Lys, D-N-Me-Lys, D-Dap, D-Dab, suitable isosteres, and corresponding D-amino acids. In some embodiments of peptide monomers described herein, Xaa¹³ is absent, or Xaa¹³ is selected from COOH and CONH₂. In at least one embodiment, Xaa¹³ is Lys. Further still in some embodiments Xaa¹³ is D-Lys. In some embodiments of the peptide dimer subunits described herein, when Xaa¹⁴ is absent, Xaa¹³ is the C-terminus; and when Xaa¹³ is the C-terminus of the subunit, Xaa¹³ may be modified to include a suitable linker moiety in accordance with the present invention.

Further, in some embodiments of the peptide monomers or dimer subunits described herein, Xaa¹⁴ is absent, or Xaa¹⁴ is selected from the group consisting of Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, COOH, CONH₂, N-Me-Lys, D-N-Me-Lys, D-Dap, D-Dab, suitable isosteres, corresponding D-amino acids, and corresponding N-Methyl amino acids. Further, in some embodiments of the peptide dimer subunits described herein, Xaa¹⁴ is absent, or Xaa¹⁴ is selected from the group consisting of Gln, Pro, Gly, His, Ala, Ile, Phe, Lys, Arg, Leu, Val, Tyr, Trp, Met, Glu, Ser, Asn, Gla, Dap, Dab, Om, D-Om, D-Lys, N-Me-Orn, N-Me-Dap, N-Me-Dab, N-Me-Lys, D-N-Me-Lys, D-Dap, D-Dab, suitable isosteres, corresponding D-amino acids, and corresponding N-Methyl amino acids. In at least one embodiment of the peptide monomers and dimer subunits described herein, Xaa¹⁴ is Lys, D-Lys, or N-Me-Lys. In some embodiments of the peptide monomer or peptide dimer subunits of the present invention, Xaa¹⁴ is Cys, HomoCys or or Pen. In some embodiments of the peptide monomer or peptide dimer subunits of the present invention, Xaa¹⁴ is Cys, D-Cys, HomoCys, Pen, or D-Pen.

In some embodiments of any of the peptide monomers or dimer subunits described herein, Xaa¹² is present, Xaa¹³ is absent, and Xaa¹⁴ is present. In particular embodiments, Xaa¹¹ is Phe(4tBu), Phe(4-COOH), Bip, 2-Nal or 1-Nal; Xaa¹² is Glu or 3-homoGlu, Xaa¹³ is absent, and Xaa¹⁴ is D-Lys or N-Me-Lys.

In at least one embodiment of the dimer subunits described herein, Xaa¹⁴ is the C-terminus, and when Xaa¹⁴ is the C-terminus of the subunit, Xaa¹⁴ may be modified to include a linker moiety in accordance with the present invention.

In at least one embodiment of peptide monomers and peptide dimer subunits, including peptide monomers and dimers of Formula (I), described herein, Xaa¹⁻¹⁴ are absent, whereby Xaa¹⁰ is the C-terminus. When Xaa¹²⁻¹⁴ are absent, Xaa¹¹ is the C-terminus. Similarly, when Xaa¹³ and Xaa¹⁴ are absent, Xaa¹² is the C-terminus. Further, when Xaa¹⁴ is absent, Xaa¹³ is the C-terminus. In some embodiments, the C-terminus of the thioether peptide monomer or dimer subunit is modified to include a suitable linker moiety (e.g. a linker moiety) or modifying group in accordance with the present invention.

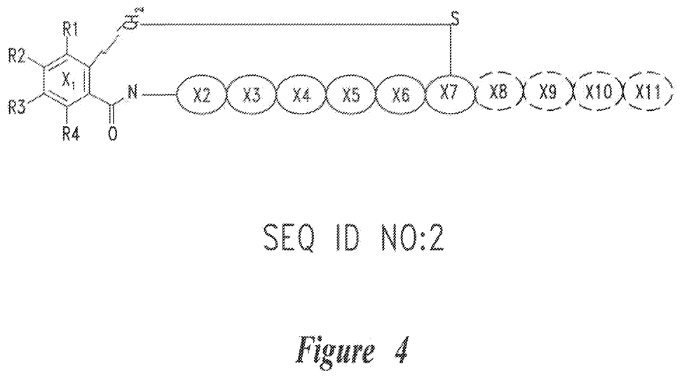

In certain embodiments of any of the peptide monomers or dimer subunits (e.g. the peptide monomers and dimers of Formula (I)) described herein, Xaa¹, Xaa² and Xaa³ are absent, and the N-terminus of the peptide comprises an aromatic group that is capable of forming a thioether bond with Xaa¹⁰. In some embodiments, Xaa⁴ comprises a 2-methylbenzoyl moiety forming an amide bond with Xaa⁵, and further comprising a methyl group forming a thioether bond with Xaa¹⁰. The 2-methylbenzoyl moiety further comprises substituent R-groups represented by R1-R4, e.g., as shown in FIG. 4.

In some instances of peptide monomers or dimers described herein, at least one substituent R-group of Xaa¹ is a free amine, whereby the N-terminus of the thioether monomer or dimer peptide of, e.g., Formula (I) or Formula (I-1), may be extended. In other instances, one or more substituent groups represented by R1-R4 is selected from the group consisting of hydrogen, a methyl group, a fluorocarbon group, a hydrocarbon, Cl, CF3, OMe, OEt, CONH₂, an aromatic group, a small pegylation group, a terminal modifying group, an acylation, a free amine, and an acid. In some embodiments, one or more substituent groups represented by R1-R4 is selected from the group consisting of hydrogen, a methyl group, a fluorocarbon group, a hydrocarbon, Cl, CF₃, OMe, OEt, CONH₂, CH₃, CH₂CH₃, an aromatic group, a small pegylation group, a terminal modifying group, an acylation, a free amine, and an acid.

In particular embodiments of any of the peptides herein, including those comprising a structure of any one of Formulas (I), (I-1), (I-2), (I-3), (V) or (I-A)-(I-I) or Formula (V), the thioether bond is in the reverse order, such that the amino acid residues and chemical moieties shown in Xaa⁴ are instead present in Xaa¹⁰, and the amino acid resides shown at Xaa¹⁰ are instead present at Xaa⁴. In this reverse orientation, the amino acid or chemical moiety at position Xaa¹⁰ is one that comprises a free amine.

In some embodiments of the peptide monomers and dimer subunits described herein, the C-terminal residue of Formula (I) or Formula (V) or any peptide monomer or peptide dimer described herein further comprises a modifying group or a suitable linker moiety, e.g., a modifying group or linker selected from the group consisting of DIG, PEG4, PEG13, PEG25, PEG1K, PEG2K, PEG4K, PEG5K, Polyethylene glycol having molecular weight from 400 Da to 40,000 Da, PEG having a molecular weight of 40,000 Da to 80,000 Da, IDA, Ac-IDA, ADA, Glutaric acid, Succinic acid, Isophthalic acid, 1,3-phenylenediacetic acid, 1,4-phenylenediacetic acid, 1,2-phenylenediacetic acid, AADA, suitable aliphatic acids, suitable aromatic acids, heteroaromatic acids. Examples of other linkers are described herein and include but are not limited to those linkers shown in Table 2.

Referring now to FIG. 4, one aspect of the present invention relates to a thioether peptide monomer or dimer (or subunit of a peptide dimer molecule) comprising the structure according to Formula (II):
Xaa¹-Xaa²-Xaa³-Xaa⁴-Xaa⁵-Xaa⁶-Xaa⁷-Xaa⁸-Xaa⁹-Xaa¹⁰-Xaa¹¹ (SEQ ID NO: 2), or a pharmaceutically acceptable salt thereof, wherein the peptide monomer or each subunit of the thioether peptide dimer comprises a thioether bond between Xaa¹ and Xaa⁷.

The N-terminus of a peptide monomer or dimer subunit represented by Formula (II) comprises an aromatic group that is capable of forming a thioether bond with Xaa⁷. In some embodiments, Xaa¹ comprises a 2-methylbenzoyl moiety forming an amide bond with Xaa², and further comprising a methyl group forming a thioether bond with Xaa⁷. The 2-methylbenzoyl moiety may further comprise substituent R-groups represented by R1-R4, e.g., as shown in FIG. 4, including those described herein.

In some instances, at least one substituent R-group of Xaa¹ is a free amine, whereby the N-terminus of the thioether peptide of Formula (II) may be extended. In other instances, one or more substituent groups represented by R1-R4 is selected from the group consisting of hydrogen, a methyl group, a fluorocarbon group, a hydrocarbon, Cl, CF3, OMe, OEt, CONH₂, an aromatic group, a small pegylation group, a terminal modifying group, an acylation, a free amine, and an acid.

For each embodiment of Formula (II) or Formula (VI), a thioether bond exists between Xaa¹ and Xaa⁷. Thus, the thioether peptide monomers and dimer subunits of the present invention are cyclized through a thioether bond. In one embodiment, Xaa⁷ is Cys. In another embodiment, preferably Xaa⁷ is Pen. In other embodiments, Xaa⁷ is D-Cys or homo-Cys.

In some embodiments of peptides (e.g. peptide monomers, dimers, or dimer subunits) described herein, Xaa¹ comprises an R group that is capable of being acylated via an acylating organic compound. In other instances, Xaa¹ of a peptide dimer subunit comprises an R group that is capable of being modified with a suitable linker moiety, whereby the N-terminuses of two peptide dimer subunits according to Formula (I) may be dimerized. In certain embodiments, Xaa¹ is a 2-methyl benzoyl moiety.

In particular embodiments of the Formula (II) or Formula (VI) peptides (e.g. peptide monomers or peptide dimers or subunits thereof) of the present invention, Xaa¹ is a modified HomoSer or a modified Ser group that is capable of forming a thioether bond with Xaa⁷ and Xaa⁷ is Cys, Pen, D-Cys, Homo Cys. The N-terminal residue further comprises a modifying group or suitable linker moiety, e.g., a modifying group or linker selected from the group consisting of DIG, PEG4, PEG13, PEG25, PEG1K, PEG2K, PEG4K, PEG5K, Polyethylene glycol having molecular weight from 400 Da to 40,000 Da, PEG having a molecular weight of 40,000 Da to 80,000 Da, IDA, Ac-IDA, ADA, Glutaric acid, Succinic acid, Isophthalic acid, 1,3-phenylenediacetic acid, 1,4-phenylenediacetic acid, 1,2-phenylenediacetic acid, AADA, suitable aliphatic acids, suitable aromatic acids, heteroaromatic acids. Examples of other linkers are described herein and include but are not limited to those shown in Table 3.

For each embodiment of Formula (II), Xaa² is selected from the group consisting of N(alpha)-Me-Arg, Arg, HArg, Dap, Dab, Arg-Me-sym, Arg-Me-asym, 4-Guan, Cit, Cav, and suitable isostere replacements. In some embodiments, Xaa² is N(alpha)Methylated. Preferably, Xaa² is N-Me-Arg. In other embodiments, preferably Xaa² is Arg.

For each embodiment of Formula (II), Xaa³ is selected from the group consisting of Ser, Gly, and suitable isostere replacements. Preferably, Xaa³ is Ser.

For each embodiment of Formula (II), Xaa⁴ is selected from the group consisting of Asp, N-Me-Asp, Asp(OMe), D-Asp, and a suitable isostere replacements. In some embodiments, Xaa⁴ is N(alpha)Methylated. In some embodiments, Xaa⁴ is Asp or N-Me-Asp. In some embodiments, Xaa⁴ is Asp.

For each embodiment of Formula (II), Xaa⁵ is selected from the group consisting of Thr, Gln, Ser, Asp, Pro, Gly, His, Ala, Ile, Phe, Lys, Arg, Asn, Glu, Val, Tyr, Trp, Leu, Met, and N-Methyl amino acids including N-Me-Thr, and suitable isostere replacements. In some embodiments, Xaa⁵ is N(alpha)Methylated. In some embodiments, Xaa⁵ is selected from the group consisting of Thr, Gln, Ser, Asp, Gly, His, Ala, Ile, Phe, Lys, Arg, Asn, Glu, Val, Tyr, Trp, Leu, Met, and N-Methyl amino acids including N-Me-Thr, and suitable isostere replacements. Preferably, Xaa⁵ is Thr.

For each embodiment of Formula (II), Xaa⁶ is selected from the group consisting of Gln, Asn, Asp, Pro, Gly, Ala, Phe, Leu, Glu, Ile, Val, HLeu, n-Butyl Ala, n-Pentyl Ala, n-Hexyl Ala, Nle, cyclobutyl-Ala, N-Me-Leu, and suitable isostere replacements. In some embodiments, Xaa⁶ is selected from the group consisting of Gln, Asn, Asp, Pro, Gly, Ala, Phe, Leu, Glu, Ile, Val, HLeu, n-Butyl Ala, n-Pentyl Ala, n-Hexyl Ala, Nle, cyclobutyl-Ala, N-Me-Leu, and suitable isostere replacements. In some embodiments, Xaa⁶ is N(alpha)Methylated. Preferably, Xaa⁶ is Leu.

For each embodiment of Formula (II), Xaa⁷ is selected from the group consisting of Cys, N-Me-Cys, D-Cys, HCys, Pen, and D-Pen. Preferably, in one embodiment Xaa⁷ is Pen. In another embodiment, Xaa⁷ is preferably Cys.

For each embodiment of Formula (II), Xaa⁸ is selected from the group consisting of Gly, Gln, Asn, Asp, Ala, Ile, Leu, Val, Met, Thr, Lys, Trp, Tyr, His, Glu, Ser, Arg, Pro, Phe, Sar, 1-Nal, 2-Nal, D-1-Nal, D-2-Nal, D-Phe, D-Tyr, HPhe, Phe(4-F), O-Me-Tyr, dihydro-Trp, Dap, Dab, Dab(Ac), Orn, D-Om, N-Me-Orn, N-Me-Dap, D-N-Me-Lys, D-Dap, D-Dab, Bip, Ala(3,3diphenyl), Biphenyl-Ala, aromatic ring substituted Phe, aromatic ring substituted Trp, aromatic ring substituted His, hetero aromatic amino acids, N-Me-Lys, N-Me-Lys(Ac), 4-Me-Phe, and corresponding D-amino acids and suitable isostere replacements. In other embodiments, Xaa⁸ is N(alpha)Methylated. Further, in some embodiments Xaa⁸ is acylated. In some embodiments of peptide monomers or peptide dimers described herein, Xaa⁸ is absent.

In particular embodiments of peptide dimer subunits of Formula (II) or Formula (VI), Xaa⁹⁻¹¹ are absent, and Xaa⁸ is the C-terminus of the subunit. When Xaa⁸ is the C-terminus of the subunit, Xaa⁸ may be modified to include a suitable linker moiety in accordance with the present invention.

In some embodiments of the peptide monomers and dimer subunits of Formula (II) or Formula (VI), Xaa⁹ is absent, or Xaa⁹ is selected from the group consisting of Glu, Amide, Lys, Gln, Pro, Gly, His, Ala, Ile, Phe, Lys, COOH, Arg, Leu, Val, Tyr, Trp, Met, Gla, Ser, Asn, D-Glu, β-HGlu, 2-Nal, 1-Nal, D-1-Nal, D-2-Nal, D-Phe, D-Tyr, D-Asp, Bip, 3-HPhe, 3-Glu, D-Tyr, D-Lys, Dap, Dab, Orn, D-Orn, N-Me-Om, N-Me-Dap, N-Me-Dab, N-Me Lys, D-N-Me-Lys D-Dap, D-Dab, suitable isosteres, and corresponding D-amino acids. In particular embodiments of peptide monomer or dimer subunits described herein, Xaa⁹ is absent or COOH. In certain embodiments, Xaa⁹ is Glu, D-Glu, β-HGlu, or Asp.

In some embodiments of peptide dimer subunits, when Xaa¹⁰ and Xaa¹¹ are absent, Xaa⁹ is the C-terminus of the subunit. When Xaa⁹ is the C-terminus of the subunit, Xaa⁹ may be modified to include a suitable linker moiety in accordance with the present invention.

For each embodiment of Formula (II) or Formula (VI), Xaa¹⁰ may be absent, or Xaa¹⁰ is selected from the group consisting of Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, D-N-Me-Lys N-Me-Lys, D-Dap, D-Dab, suitable isosteres, and corresponding D-amino acids. In at least one embodiment, Xaa¹⁰ is Lys. Further still in some embodiments Xaa¹⁰ is D-Lys. In particular embodiments of peptide monomers or peptide dimers described herein, Xaa¹⁰ is COOH or CONH₂.

In certain embodiments of peptide monomers or peptide dimer subunits comprising Formula (II) or Formula (VI), when Xaa¹¹ is absent, Xaa¹⁰ is the C-terminus. When Xaa¹⁰ is the C-terminus of the subunit, Xaa¹⁰ may be modified to include a suitable linker moiety in accordance with the present invention. Further, in some embodiments, Xaa¹¹ is absent, or selected from the group consisting of Gln, Pro, Gly, His, Ala, Ile, Phe, Lys, Arg, Leu, Val, Tyr, Trp, Met, Glu, Ser, Asn, Gla, Dap, Dab, Om, D-Orn, D-Lys, N-Me-Orn, N-Me-Dap, N-Me-Dab, D-N-Me-Lys N-Me-Lys, D-Dap, D-Dab, suitable isosteres, and corresponding D-amino acids. In at least one embodiment, Xaa¹⁰ is Lys. Further still in some embodiments Xaa¹⁰ is D-Lys. In some embodiments of peptide monomers, Xaa¹⁰ is COOH or CONH₂.

In certain embodiments of peptide monomers or peptide dimer subunits, Xaa¹¹ is the C-terminus. When Xaa¹¹ is the C-terminus of the subunit, Xaa¹¹ may be modified to include a linker moiety in accordance with the present invention.

In at least one embodiment of peptide monomers of the present invention, Xaa⁸⁻¹¹ are absent, whereby Xaa⁷ is the C-terminus.

In particular embodiments of peptide monomer and dimer subunits comprising Formula (II), when Xaa⁹⁻¹¹ are absent, Xaa⁸ is the C-terminus. Similarly, in certain embodiments, when Xaa¹⁰ and Xaa¹¹ are absent, Xaa⁹ is the C-terminus. Further, when Xaa¹¹ is absent, Xaa¹⁰ is the C-terminus. In some embodiments, the C-terminus of the thioether peptide is modified to include a modifying group in accordance with the present invention. In some embodiments, the C-terminus of the thioether peptide monomer or dimer subunit comprises NH₂ or OH.

In particular embodiments of any of the peptides herein, including those comprising a structure of any one of Formulas (II), (II-A), (A), (III), or (IV) or Formula (VI), the thioether bond is in the reverse order, such that the amino acid residues and chemical moieties shown in Xaa¹ are instead present in Xaa⁷, and the amino acid resides shown at Xaa⁷ are instead present at Xaa¹. In this reverse orientation, the amino acid or chemical moiety at position Xaa⁷ is one that comprises a free amine.

In certain embodiments peptides comprising Formula (II) or Formula (VI):
Xaa¹ is a 2-Me-benzoyl group capable of forming a thioether bond with Xaa⁷;
Xaa² is selected from the group consisting of N(alpha)-Me-Arg, Arg, HArg, Dap, Dab, Arg-Me-sym, Arg-Me-asym, 4-Guan, Cit, Cav, and suitable isostere replacements;
Xaa³ is selected from the group consisting of Ser, Gly, and suitable isostere replacements;
Xaa⁴ is selected from the group consisting of Asp, N-Me-Asp, Asp(OMe), D-Asp, and a suitable isostere replacements;
Xaa⁵ is selected from the group consisting of Thr, Gln, Ser, Asp, Pro, Gly, His, Ala, Ile, Phe, Lys, Arg, Asn, Glu, Val, Tyr, Trp, Leu, Met, and N-Methyl amino acids including N-Me-Thr, and suitable isostere replacements;
Xaa⁶ is selected from the group consisting of Gln, Asn, Asp, Pro, Gly, Ala, Phe, Leu, Glu, Ile, Val, HLeu, n-Butyl Ala, n-Pentyl Ala, n-Hexyl Ala, Nle, cyclobutyl-Ala, N-Me-Leu, and suitable isostere replacements;
Xaa⁷ is selected from the group consisting of Cys, N-Me-Cys, D-Cys, HCys, Pen, and D-Pen;
Xaa⁸ is selected from the group consisting of absent, Gly, Gln, Asn, Asp, Ala, Ile, Leu, Val, Met, Thr, Lys, Trp, Tyr, His, Glu, Ser, Arg, Pro, Phe, Sar, 1-Nal, 2-Nal, HPhe, Phe(4-F), O-Me-Tyr, dihydro-Trp, Dap, Dab, Dab(Ac), Orn, D-Orn, N-Me-Orn, N-Me-Dap, D-Dap, D-Dab, Bip, Ala(3,3diphenyl), Biphenyl-Ala, aromatic ring substituted Phe, aromatic ring substituted Trp, aromatic ring substituted His, hetero aromatic amino acids, N-Me-Lys, N-Me-Lys(Ac), 4-Me-Phe, and corresponding D-amino acids and suitable isostere replacements;
Xaa⁹ is selected from the group consisting of absent, Glu, Amide, Lys, COOH, CONH₂, Gln, Pro, Gly, His, Ala, Ile, Phe, Lys, Arg, Leu, Val, Tyr, Trp, Met, Gla, Ser, Asn, D-Glu, β-HGlu, 2-Nal, 1-Nal, D-Asp, Bip, β-HPhe, β-Glu, D-Tyr, D-Lys, Dap, Dab, Orn, D-Orn, N-Me-Orn, N-Me-Dap, N-Me-Dab, N-Me Lys, D-Dap, D-Dab, suitable isosteres, and corresponding D-amino acids;
Xaa¹⁰ is selected from the group consisting of absent, Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, N-Me-Lys, D-Dap, D-Dab, COOH, CONH₂, suitable isosteres, and corresponding D-amino acids; and
Xaa¹¹ is selected from the group consisting of absent, Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, N-Me-Lys, D-Dap, D-Dab, COOH, CONH₂, suitable isosteres, and corresponding D-amino acids, wherein the peptide further comprises a thioether bond between Xaa¹ and Xaa⁷.

Another aspect of the present invention relates to a thioether peptide monomer or each subunit of a dimer compound comprising the structure according to Formula (II-A) (SEQ ID NO: 45),
Xaa¹-Xaa²-Xaa³-Xaa⁴-Xaa⁵-Xaa⁶-Xaa⁷-Xaa⁸-Xaa⁹-Xaa¹⁰-Xaa¹¹ (Formula II-A)), or a pharmaceutically acceptable salt thereof, wherein the peptide comprises a thioether bond between Xaa¹ and Xaa⁷, wherein
Xaa¹ (or the N-terminus) of the peptide represented by Formula (II-A) comprises a group, e.g., optionally an aromatic group, that is capable of forming a thioether bond with Xaa⁷. In some embodiments, Xaa¹ comprises a 2-methylbenzoyl moiety forming an amide bond with Xaa², and further comprising a methyl group forming a thioether bond with Xaa⁷. The 2-methylbenzoyl moiety further comprises substituent R-groups represented by R1-R4; in some instances, at least one substituent R-group of Xaa¹ is a free amine, whereby the N-terminus of the thioether peptide of Formula (II-A) may be extended; in other instances, one or more substituent groups represented by R1-R4 is selected from the group consisting of hydrogen, a methyl group, a fluorocarbon group, a hydrocarbon, Cl, CF3, OMe, OEt, CONH₂, an aromatic group, a small pegylation group, a terminal modifying group, an acylation, a free amine, and an acid. In particular embodiments, Formula (II-A) is directed to a peptide monomer or peptide dimer subunit and Xaa¹ is a modified Ser or a modified Homo-Ser, e.g., Homo-Ser-Cl. In some embodiments, Formula (II-A) is directed to a peptide dimer subunit and Xaa⁴ is modified Homo-Ser, and Xaa¹⁰ is Cys, D-Cys, or HomoCys.

For each embodiment of Formula (II-A), Xaa² is selected from the group consisting of N(alpha)-Me-Arg, Arg, HArg, Dap, Dab, Arg-Me-sym, Arg-Me-asym, 4-Guan, Cit, Cav, and suitable isostere replacements. In some embodiments, Xaa² is N(alpha)Methylated. Preferably, Xaa² is N-Me-Arg. In other embodiments, preferably Xaa² is Arg.

For each embodiment of Formula (II-A), Xaa³ is selected from the group consisting of Ser, Gly, Thr, Ile and suitable isostere replacements. Preferably, Xaa³ is Ser.

For embodiments of Formula (II-A) directed to peptide monomers, Xaa⁴ is selected from the group consisting of Asp, N-Me-Asp, Asp(OMe), D-Asp, and a suitable isostere replacements. For embodiments of Formula (II-A), Xaa⁴ is selected from the group consisting of Asp, N-Me-Asp, D-Asp, and a suitable isostere replacements. In some embodiments of peptide monomers and dimer subunits, Xaa⁴ is N(alpha)Methylated. Preferably, Xaa⁴ is Asp.

For each embodiment of Formula (II-A), Xaa⁵ is selected from the group consisting of Thr, Gln, Ser, Asp, Pro, Gly, His, Ala, Ile, Phe, Lys, Arg, Asn, Glu, Val, Tyr, Trp, Leu, Met, and N-Methyl amino acids including N-Me-Thr, and suitable isostere replacements. In some embodiments, Xaa⁵ is N(alpha)Methylated. Preferably, Xaa⁵ is Thr.

For each embodiment of Formula (II-A), Xaa⁶ is selected from the group consisting of Gln, Asn, Asp, Pro, Gly, Ala, Phe, Leu, Glu, Ile, Val, HLeu, n-Butyl Ala, n-Pentyl Ala, n-Hexyl Ala, Nle, cyclobutyl-Ala, N-Me-Leu, and suitable isostere replacements. In some embodiments, Xaa⁶ is N(alpha)Methylated. In some embodiments, Xaa⁶ is Leu.

For each embodiment of Formula (II-A), Xaa⁷ is selected from the group consisting of Cys, N-Me-Cys, D-Cys, HCys, Pen, D-Pen and Pen(═O). Preferably, in one embodiment Xaa⁷ is Pen. In another embodiment, Xaa⁷ is preferably Cys. In particular embodiments of peptides (e.g. peptide monomers, dimers or subunits thereof) of Formula (II-A), Xaa⁷ is capable of forming a thioether bond with Xaa¹. In some embodiments of peptides (e.g. peptide monomers, dimers or subunits thereof) of Formula (II-A), Xaa⁷ is Cys, D-Cys or HomoCys.

For each embodiment of Formula (II-A), Xaa⁸ is absent, or Xaa⁸ is selected from the group consisting of Gly, Gln, Asn, Asp, Ala, Ile, Leu, Val, Met, Thr, Lys, Trp, Tyr, His, Glu, Ser, Arg, Pro, Phe, Sar, 1-Nal, 2-Nal, D-1-Nal, D-2-Nal, D-Phe, D-Tyr, HPhe, Phe(4-F), O-Me-Tyr, dihydro-Trp, Dap, Dab, Dab(Ac), Om, D-Orn, N-Me-Om, N-Me-Dap, D-N-Me-Lys, D-Dap, D-Dab, Bip, Ala(3,3diphenyl), Biphenyl-Ala, Phe(4tBu), Phe(4-OMe), Phe(4-COOH), Phe(2-carbomyl), Phe(3-carbomyl), Phe(CF3), Phe(2,4-diCl), Phe(3,4-diCl), Aic, N-Me-Tyr, N-Me-Phe, Tic, Phe(4CF3), aromatic ring substituted Phe, aromatic ring substituted Trp, aromatic ring substituted His, hetero aromatic amino acids, N-Me-Lys, N-Me-Lys(Ac), 4-Me-Phe, and corresponding D-amino acids and suitable isostere replacements. In other embodiments, Xaa⁸ is N(alpha)Methylated. Further, in some embodiments Xaa⁸ is acylated.

In some embodiments of Formula (II-A), Xaa⁹ is absent, or Xaa⁹ is selected from the group consisting of Glu, Amide, Lys, COOH, Gln, Pro, Gly, His, Ala, Ile, Phe, Lys, Arg, Leu, Val, Tyr, Trp, Met, Gla, Ser, Asn, D-Glu, β-HGlu, 2-Nal, 1-Nal, D-1-Nal, D-2-Nal, D-Phe, D-Tyr, D-Asp, Bip, β-HPhe, β-Glu, D-Tyr, D-Lys, Dap, Dab, Orn, D-Orn, N-Me-Orn, N-Me-Dap, N-Me-Dab, N-Me Lys, D-N-Me-Lys D-Dap, D-Dab, O-Me-Glu, suitable isosteres, and corresponding D-amino acids. Preferably, Xaa⁹ is Glu, D-Glu, β-HGlu, Asp, D-His, F(4-COOH), Tic, D-Trp, D-Leu, D-Arg, D-Thr.

For particular embodiments of Formula (II-A), Xaa¹⁰ may be absent or any amino acid. For certain embodiments, Xaa¹⁰ may be absent or Xaa¹⁰ is selected from the group consisting of Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, D-N-Me-Lys N-Me-Lys, D-Dap, D-Dab, COOH, CONH₂, suitable isosteres, and corresponding D-amino acids. In at least one embodiment, Xaa¹⁰ is Lys. Further still in some embodiments Xaa¹⁰ is D-Lys.

Further, in particular embodiments of Formula (II-A) directed to peptide monomers, Xaa¹¹ is absent or any amino acid. In certain embodiments directed to peptide monomers, Xaa¹¹ is selected from the group consisting of Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, D-N-Me-Lys, N-Me-Lys, D-Dap, D-Dab, COOH, CONH₂, suitable isosteres, and corresponding D-amino acids. In at least one embodiment, Xaa¹¹ is Lys. Further still in some embodiments Xaa¹¹ is D-Lys.

In particular embodiments of Formula (II-A) directed to peptide dimer subunits, Xaa¹¹ is absent or selected from the group consisting of Gln, Pro, Gly, His, Ala, Ile, Phe, Lys, Arg, Leu, Val, Tyr, Trp, Met, Glu, Ser, Asn, Gla, Dap, Dab, Orn, D-Orn, D-Lys, N-Me-Orn, N-Me-Dap, N-Me-Dab, D-N-Me-Lys, N-Me-Lys, D-Dap, D-Dab, Cys, HomoSys, Pen, suitable isosteres, and corresponding D-amino acids, and amino acids comprising a free amine group. In at least one embodiment, Xaa¹¹ is Lys. Further still in some embodiments Xaa¹¹ is D-Lys. In at least one embodiment, Xaa¹¹ is the C-terminus. When Xaa¹¹ is the C-terminus of the subunit, Xaa¹¹ may be modified to include a linker moiety in accordance with the present invention.

In particular embodiments of Formula (II-A), Xaa⁹ is not O-Me-Glu, and it absent or selected from from the group consisting of Glu, Amide, Lys, COOH, Gln, Pro, Gly, His, Ala, Ile, Phe, Lys, Arg, Leu, Val, Tyr, Trp, Met, Gla, Ser, Asn, D-Glu, β-HGlu, 2-Nal, 1-Nal, D-1-Nal, D-2-Nal, D-Phe, D-Tyr, D-Asp, Bip, β-HPhe, β-Glu, D-Tyr, D-Lys, Dap, Dab, Orn, D-Om, N-Me-Om, N-Me-Dap, N-Me-Dab, N-Me Lys, D-N-Me-Lys D-Dap, D-Dab, O-Me-Glu, suitable isosteres, and corresponding D-amino acids.

In particular embodiments of peptide monomers and dimer subunits, e.g. m those of Formula (II) or (VI), Xaa⁸⁻¹¹ are absent, whereby Xaa⁷ is the C-terminus. When Xaa⁹⁻¹¹ are absent, Xaa⁸ is the C-terminus. Similarly, when Xaa¹⁰ and Xaa¹¹ are absent, Xaa⁹ is the C-terminus. Further, when Xaa¹¹ is absent, Xaa¹⁰ is the C-terminus. In certain embodiments, Xaa⁸⁻¹⁰ are absent, and Xaa¹¹ is the C-terminus. In certain embodiments, Xaa⁸ is present, Xaa⁹⁻¹⁰ are absent and Xaa¹¹ is the C-terminus. In certain embodiments, Xaa⁸ and Xaa⁹ are present, Xaa¹⁰ is absent and Xaa¹¹ is the C-terminus. In some embodiments of peptide monomers or dimers, the C-terminus of the thioether peptide is modified to include a modifying group or linker in accordance with the present invention.

For certain embodiments of Formula (II-A), a thioether bond exists between Xaa¹ and Xaa⁷. Thus, the thioether peptides of the present invention may be cyclized through a thioether bond. In one embodiment, Xaa⁷ is Cys. In another embodiment, preferably Xaa⁷ is Pen. In other embodiments, Xaa⁷ is D-Cys or homo-Cys. In certain embodiments, Xaa¹ is Homo-Ser-Cl, and Xaa⁷ is Cys, D-Cys or HomoCys.

In some embodiments of peptide monomer, the C-terminal residue of Formula (II) or (II-A) further comprises a modifying group selected from the group consisting of DIG, PEG4, PEG13, PEG25, PEG1K, PEG2K, PEG4K, PEG5K, Polyethylene glycol having molecular weight from 400 Da to 40,000 Da, PEG having a molecular weight of 40,000 Da to 80,000 Da, IDA, Ac-IDA, ADA, Glutaric acid, Isophthalic acid, 1,3-phenylenediacetic acid, 1,4-phenylenediacetic acid, 1,2-phenylenediacetic acid, AADA, suitable aliphatic acids, suitable aromatic acids, heteroaromatic acids. In some embodiments, the C-terminus of the thioether peptide comprises NH₂ or OH.

Some embodiments of the peptide monomers of the present invention comprise a peptide molecule comprising an N(alpha)-Me-Arg residue, as represented by at least one of SEQ ID NOs: 1-32.

In one embodiment, a thioether peptide of the present invention comprises one or two peptide dimer subunits or a peptide monomer of Formula (A) (SEQ ID NO: 48):

(Formula (A))
Xaa¹-Xaa²-Xaa³-Xaa⁴-Xaa⁵-Xaa⁶-Xaa⁷-Xaa⁸⁻Xaa⁹-
Xaa¹⁰,

or a pharmaceutically acceptable salt thereof, wherein
Xaa¹ comprises an aromatic group capable of forming a thioether bond with Xaa⁷, such as a 2-methylbenzoyl moiety;
Xaa² is N-methyl-Arg;
Xaa³ is Ser, Gly, Thr, or Ile; and
wherein in some embodiments if Formula (A) is directed to a peptide monomer then Xaa³ is Ser, Gly, Thr, or Ile; and
wherein in other embodiments if Formula (A) is directed to a peptide dimer subunit then Xaa³ is Ser; and
Xaa⁴ is Asp;
Xaa⁵ is Thr;
Xaa⁶ is Leu or Nle;
Xaa⁷ is Cys, D-Cys, Hcys, or Pen;
Xaa⁸ is Trp, Tic, Bip, 1-Nal, 2-Nal, Phe(4tBu), or Phe(4-COOH);
Xaa⁹ is Glu, β-homo-Glu, or D-Glu;
Formula (A) is directed to a peptide monomer and Xaa¹⁰ is any amino acid; or Formula (A) is directed to a peptide dimer subunit, and Xaa¹⁰ is Lys, D-Lys, N-Me-Lys or D-N-Me-Lys; and
wherein the peptide molecule comprises a thioether bond between Xaa¹ and Xaa⁷.

In particular embodiments of Formula (A), Xaa¹⁰ is D-Lys or N-Me-Lys.

In certain embodiments, Xaa¹⁰ or the C-terminus of the peptide comprises an NT-2 or an OH.

In certain embodiments of peptide monomers, a free amine in the C-terminal amino acid is capped, e.g., with an acetyl group.

Illustrative thioether peptide dimers (and subunits thereof) and peptide monomers of the present invention are shown in the accompanying figures and sequence listing.

In certain embodiments, a thioether peptide monomer, dimer or peptide subunit of a dimer, optionally a homodimer, of the present invention comprises Formula (III) (SEQ ID NO: 46):

(Formula (III)
Xaa¹-Xaa²-Xaa³-Xaa⁴-Xaa⁵-Xaa6-Xaa⁷-Xaa⁸-Xaa⁹-
Xaa¹⁰

or a pharmaceutically acceptable salt thereof, wherein the thioether peptide comprises a thioether bond between Xaa¹ and Xaa⁷ in the peptide monomer or in one or both peptide monomer subunits, wherein the two subunits of Formula (III) of a peptide dimer are dimerized at their C-termini via a linker, e.g., DIG, and wherein
Xaa¹ is 2-methylbenzoyl;
Xaa² is N-Me-Arg;
Xaa³ is Ser, Gly, Thr, or Ile; or
Xaa⁴ is Asp;
Xaa⁵ is Thr; and
Xaa⁶ is Leu or Nle; or
Xaa⁷ is Pen, Cys or d-Cys; or
Xaa⁸ is Phe, D-Phe, Tyr, Bip, Tic, 1-Nal, 2-Nal, or Trp;
Xaa⁹ is D-Glu, Glu, Tyr, b-homo-Glu, or 2-Nal; and
Xaa¹⁰ is D-Lys, N-Me-D-Lys, Dap, Phe, D-Phe or absent.

In certain embodiments, Formula (III) is directed to a peptide monomer wherein:
Xaa¹ is 2-methylbenzoyl;
Xaa² is N-Me-Arg;
Xaa³ is Ser, Gly, Thr, or Ile;
Xaa⁴ is Asp;
Xaa⁵ is Thr;
Xaa⁶ is Leu or Nle;
Xaa⁷ is Pen, Cys or d-Cys;
Xaa⁸ is Phe, D-Phe, Tyr, 1-Nal, 2-Nal, or Trp;
Xaa⁹ is D-Glu, Glu, Tyr, b-homo-Glu, or 2-Nal; and
Xaa¹⁰ is D-Lys, N-Me-D-Lys, Dap, Phe, D-Phe or absent.

In certain embodiments, Formula (III) is directed to a peptide dimer subunit wherein:
Xaa¹ is 2-methylbenzoyl;
Xaa² is N-Me-Arg;
Xaa³ is Ser;
Xaa⁴ is Asp;
Xaa⁵ is Thr;
Xaa⁶ is Leu;
Xaa⁷ is Pen or, Cys;
Xaa⁸ is Phe, Tyr, Bip, Tic, 2-Nal, or Trp;
Xaa⁹ is D-Glu; and
Xaa¹⁰ is D-Lys.

In certain embodiments of peptide monomers, Xaa¹⁰ is acetylated or comprises a modifying group, e.g., PEG8.

In certain embodiments, the C-terminus of a peptide monomer or subunit of a peptide dimer comprises an NH₂ or an OH. In particular embodiments, the C-terminus of a peptide dimer subunit comprises an NH₂ or an OH either before or after dimerization.

In certain embodiments, a thioether peptide, e.g. a peptide monomer or peptide dimer, optionally a homodimer, of the present invention comprises Formula (IV) (SEQ ID NO: 47):

(Formula (IV))
Xaa¹-Xaa²-Xaa³-Xaa⁴-Xaa⁵-Xaa6-Xaa⁷⁻Xaa⁸-Xaa⁹-
Xaa¹⁰

or a pharmaceutically acceptable salt thereof, wherein the thioether peptide comprises a thioether bond between Xaa¹ and Xaa⁷ in the peptide monomer or in one or both peptide subunits of a peptide dimer, wherein the two subunits of Formula (IV) are dimerized at their C-termini via a linker, e.g., DIG, and wherein
Xaa¹ is 2-methylbenzoyl;
Xaa² is N-Me-Arg;
Xaa³ is Ser;
Xaa⁴ is Asp;
Xaa⁵ is Thr;
Xaa⁶ is Leu or Nle;
Xaa⁷ is Pen, Cys, homoCys, Pen(═O), or D-Cys; wherein in certain embodiments, if Formula (IV) is directed to a peptide monomer, then Xaa⁷ is Pen, Cys, homoCys, or D-Cys;
Xaa⁸ is Phe, D-Phe, Tyr, D-Tyr, His, Bip, Tic, 1-Nal, 2-Nal, F(CH3), F(2,4-diCl), F(3,4-diCl), Aic, N-Me-Tyr, N-Me-Phe, F(2-carbomyl), F(3-carbomyl), F(4-COOH), F(4OMe), F(4tBu), F-(4-F), F(4CF3), or Trp; and
Xaa⁹ is absent, Glu, β-homo-Glu, Bip, O-Me-Glu, D-Lys, D-Phe, Tyr, 2-Nal, D-Tyr, Pro, Tic, D-Glu, D-Thr, D-Arg, D-Leu, D-Trp, F(4-COOH), D-His, Pro, D-Pro, or E(OMe); wherein in some embodiments, if Formula (IV) is directed to a peptide dimer subunit, then Xaa⁹ is Glu, β-homo-Glu, Bip, O-Me-Glu, D-Lys, D-Phe, Tyr, 2-Nal, D-Tyr, Pro, Tic, D-Glu, D-Thr, D-Arg, D-Leu, D-Trp, F(4-COOH), D-His, Pro, D-Pro, or E(OMe);
wherein in some embodiments, if Formula (IV) is directed to a peptide monomer, then Xaa¹⁰ is absent or any amino acid residue; and
wherein in other embodiments, if Formula (IV) is directed to a peptide dimer subunit, then Xaa¹⁰ is D-Lys, N-Me-Lys, N-Me-D-Lys, Lys, Dap, Dab, D-Dab, D-Dap, Orn N-Me-Om, D-Orn.

In certain embodiments of the peptide monomer or peptide dimer, Xaa¹⁰ or the C-terminal amino acid does not comprise a free amine. In particular embodiments of the peptide monomer or peptide dimer, Xaa¹⁰ is D-Lys, N-Me-Lys, N-Me-D-Lys, Dap, Phe, Ser, Glu, or absent.

In certain embodiments of Formulas (II), (II-A), (A), (III), (IV), (VI) or Formula (VI), Xaa⁸ may also be Bpa, Phe(3-Me), Phe(2-Me), Phe(2-CF3), or P-Me-Phe.

In certain embodiments of Formulas (II), (II-A), (A), (III), (IV), (VI) or Formula (VI), Xaa⁹ may also be N-Me-Glu, N-Me-Asp, or alpha-H-Glu.

In certain embodiments of Formulas (II), (II-A), (A), (III), (IV), (VI) or Formula (VI), e.g., when the peptide is a dimer, Xaa¹⁰ is selected from the group consisting of: Lys, D-Lys, N-Me-Lys, D-N-Me-Lys, Orn, Dab, Dap, Homo-Lys, D-Dap, D-Dab, Cys, HomoCys, Pen, or D-Orn, while in other embodiments, Xaa¹⁰ is selected from D-Lys, N-Me-Lys, and D-N-Me-Lys.

In certain embodiments of the peptide monomers or peptide dimers described herein, the N-terminus of the peptide is acylated.

In certain embodiments of the peptide monomers and dimer subunits, Xaa¹⁰ or the C-terminus of each peptide or peptide subunit comprises an NH₂ or an OH. In certain embodiments of the peptide dimer subunits, the C-terminus of comprises an NH₂ or an OH either before or after dimerization.

In certain embodiments of peptide monomers described herein, a free amine in the C-terminal amino acid is capped, e.g., with an acetyl group.

Particular aspects of the present invention relate to peptide inhibitors of α4β7 comprising the following core consensus sequence (shown left to right from N-term to C-term):

(SEQ ID NO: 390)
Y-(N-Me-Arg)-Ser-Glu-Thr-Leu-X

wherein Y is a 2-methyl benzoyl moiety capable of forming a thioether bond with X, and wherein X is an amino acid residue selected from Pen, Cys, D-Cys and HomoCys. In particular embodiments, X is Pen. In particular embodiments, the core sequence comprises an intramoleculer thioether bond between X and Y. In particular embodiments, the peptide inhibitor is a monomer. In particular embodiments, the peptide inhibitor is a dimer comprising two peptide monomer subunits, each comprising this core sequence. In particular embodiments, the monomer peptide inhibitor comprises 7-15 amino acid residues. In particular embodiments, each monomer subunit of the dimer peptide inhibitor comprises 7-15 amino acid residues. In certain embodiments, the two monomer subunits are linker via their respective N- or C-termini. In particular embodiments, they are linker by each of their C-termini. In certain embodiments, the peptide inhibitor further comprises an aromatic amino acid immediately downstream of X. In particular embodiments, any of the peptides described herein may comprise this core sequence.

In some embodiments, the N- or C-terminal residue of Formula (I), Formula (II), Formula (III), Formula (IV), Formula (V), Formula (VI) Formula (I-A), Formula (II-A), Formula (A), or any of the other peptide monomers or peptide subunits of dimer molecules described herein, further comprises a modifying group or suitable linker moiety selected from the group consisting of DIG, PEG4, PEG13, PEG25, PEG1K, PEG2K, PEG4K, PEG5K, Polyethylene glycol having molecular weight from 400 Da to 40,000 Da, PEG having a molecular weight of 40,000 Da to 80,000 Da, IDA, Ac-IDA, ADA, Glutaric acid, Isophthalic acid, 1,3-phenylenediacetic acid, 1,4-phenylenediacetic acid, 1,2-phenylenediacetic acid, AADA, suitable aliphatic acids, suitable aromatic acids, heteroaromatic acids.

Particular embodiments of the present invention relate to a peptide dimer comprising a linker. When the linker is IDA, ADA or any linker with free amine, it can be acylated with acylating organic compound selected from the group consisting of 2-me-Trifluorobutyl, Trifluoropentyl, Acetyl, Octonyl, Butyl, Pentyl, Hexyl, Palmityl, Lauryl, Oleoyl, Lauryl, Trifluoromethyl butyric, cyclopentane carboxylic, cyclopropylacetic, 4-fluorobenzoic, 4-fluorophenyl acetic, 3-Phenylpropionic, tetrahedro-2H-pyran-4carboxylic, succinic acid, and glutaric acid, straight chain aliphatic acids with 10 to 20 carbon units, cholic acid and other bile acids. In some instances small PEG (PEG4-PEG13), Glu, Asp, is used as spacer before acylations.

Some embodiments of the present invention comprise a peptide monomer or dimer molecule comprising an N(alpha)-Me-Arg residue, as represented by at least one of SEQ ID NOs: 1-23.

In certain embodiments, a peptide monomer or at least one subunit of a peptide dimer molecule of the present invention comprises, consists essentially of, or consists of an amino acid sequence or structure described herein, including any of the amino acid sequences shown in the accompanying sequence listing or figures, with or without any indicated N- or C-terminal modifications, linkers or modifying group. In certain embodiments, a peptide dimer molecule of the present invention comprises two peptide monomer subunits, each having an amino acid sequence or structure described herein, including any of the amino acid sequences shown in the accompanying sequence listing or figures, with or without any indicated N- or C-terminal modifications, linkers or modifying group. In particular embodiments, a peptide monomer or one or both of the peptide monomer subunits present in a peptide dimer molecule includes a thioether intramolecular linkage, e.g., a thioether bond between two amino acids within the peptide or subunit. In particular embodiments, the peptide subunits of a peptide dimer molecule are dimerized via their N- or C-termini, e.g., using a suitable linker such as DIG.

In certain embodiments of the peptide dimer molecules, the present invention includes a peptide subunit comprising, consisting essentially of, or consisting of an amino acid sequence or structure described herein, including any of the amino acid sequences shown in the accompanying sequence listing or figures, with or without any indicated N- or C-terminal modifications, linkers or modifying group. In certain embodiments, the peptide subunit includes a thioether intramolecular linkage, e.g., a thioether bond between two amino acids within the peptide subunit. In particular embodiments, the peptide monomer subunit comprises a linker moiety, e.g., DIG, at it N- or C-termini.

In certain embodiments of any of the peptide monomers or dimer peptide subunits described herein, including those of Formula (I)-(VI) and Tables 4 and 5, or of the figures herein, the peptide monomer or subunit comprises a thioether bond. In certain embodiments, with respect to Formula (I) or (V), the thioether bond exists between Xaa⁴ and Xaa¹⁰, wherein with respect to Formulas (II)-(IV) and (VI), the thioether bond exists between Xaa¹ and Xaa⁷. In certain embodiments, the thioether is formed between a 2-methyl benzoyl moiety (e.g., at Xaa⁴ in Formula (I) or Xaa¹ in Formula (II)) and either Pen or Cys (e.g., at Xaa¹⁰ in Formula (I) or Xaa⁷ in Formula (II)). In particular embodiments, the 2-methyl benzoyl moiety forms an amide bond with an adjacent amino acid residue and comprises a methyl group forming a thioether bond with the Pen or Cys residue.

In particular embodiments of any of the various Formulas described herein, peptides having the same structure or sequence as disclosed in any one or more of PCT/US2013/064439, PCT/US2014/032391 or PCT/US2014/032392 are excluded. In other embodiments of the present invention, the peptides comprise a sequence or structure set forth in any of PCT/US2013/064439, PCT/US2014/032391 or PCT/US2014/032392.

### Peptide Molecule Structure and Biological Activity

The present invention provides various novel antagonist peptide monomers and peptide dimers, including peptide monomers and dimer molecule subunits which are cyclized through a thioether bond. These peptide molecules have been tested to more clearly characterize the increased affinity for α4β7 binding, increased selectivity against α4β1, and increased stability in simulated intestinal fluid (SIF) as well as in gastric environment under reduced conditions. These novel antagonist molecules demonstrate high binding affinity with α4β7, thereby preventing binding between α4β7 and the MAdCAM ligand. Accordingly, these peptide molecules have shown to be effective in eliminating and/or reducing the inflammation process in various experiments.

The present invention thus provides various thioether peptide monomer and dimer molecules which bind or associate with the α4β7 integrin, e.g., in serum, SIF, or SGF, to disrupt or block binding between α4β7 and the MAdCAM ligand. Some peptide monomer or peptide subunits of the invention may be constructed solely of natural amino acids. Alternatively, the peptide monomer and dimer molecules may include non-natural amino acids including, but not limited to, modified amino acids and suitable aromatic acid groups, namely a 2-methylbenzoyl moiety. Modified amino acids include natural amino acids which have been chemically modified to include a group, groups, or chemical moiety not naturally present on the amino acid. The thioether peptide monomer and dimer molecules of the present invention may additionally include D-amino acids.

In certain embodiments, peptide dimer and monomer molecules of the present invention inhibit or reduce binding between between α4β7 and the MAdCAM ligand. In certain embodiments, a peptide of the present invention reduces binding of α4β7 and the MAdCAM ligand by at least 20%, at least 30%, at least 40%, at least 50%, at least 60%, at least 70%, at least 80%, or at least 90% as compared to a negative control peptide. Methods of determining binding are known in the art and described herein, and include ELISA assays, for example.

In certain embodiments, a peptide monomer or dimer molecule of the present invention has an IC50 of <500 nM, <250 nM, <100 nM, <50 nM, <25 nM, or <10 nM. Methods of determining activity are known in the art and include any of those described in the accompanying Examples.

Some antagonist thioether cyclized peptide monomer and dimer molecules have been shown to be gastrointestinal stable and provide high levels of specificity and affinity for the α4β7 integrin. Some implementations of the present invention provide a peptide monomer or dimer molecule comprising a half-life of greater than 180 minutes when exposed to simulated intestinal fluids (SIF). Some implementations further provide a thioether peptide monomer or dimer molecule comprising a half-life from approximately 1 minute to approximately 180 minutes. Similarly these peptides are stable to gastric environment under reduced conditions with half-life >120 min when tested in DTT (Dithiothreitol) assay.

In certain embodiments, a peptide monomer or dimer molecule of the present invention has increased stability, increased gastrointestinal stability, or increased stability in stimulated intestinal fluid (SIF), as compared to a control peptide. In particular embodiments, a control peptide is a peptide having the identical or a highly related amino acid sequence (e.g., >90% sequence identity) as the peptide monomer or dimer molecule, but which does not form a cyclized structure through a thioether bond. In some embodiments relating to dimer molecules, the control peptide is not dimerized. In particular embodiments, the only difference between the peptide monomer or dimer molecule and the control peptide is that the peptide comprises one or more amino acid substitutions that introduce one or more amino acid residues into the peptide, wherein the introduced residue(s) forms a thioether bond with another residue in the peptide.

Methods of determining the stability of a peptide are known in the art. In certain embodiments, the stability of a peptide (e.g. a peptide monomer or dimer as described herein) is determined using an SIF assay, e.g., as described in the accompanying Examples. In particular embodiments, a peptide monomer or dimer molecule of the present invention has a half-life under a given set of conditions (e.g., temperature) of greater than 1 minute, greater than 10 minutes, greater than 20 minutes, greater than 30 minutes, greater than 60 minutes, greater than 90 minutes, greater than 120 minutes, greater than 3 hours, or greater than four hours when exposed to SIF. In certain embodiments, the temperature is about 25° C., about 4° C., or about 37° C., and the pH is a physiological pH, or a pH about 7.4.

In some embodiments, the half-life is measured in vitro using any suitable method known in the art, e.g., in some embodiments, the stability of a peptide monomer or dimer molecule of the present invention is determined by incubating the peptide with pre-warmed human serum (Sigma) at 37° C. Samples are taken at various time points, typically up to 24 hours, and the stability of the sample is analyzed by separating the peptide monomer or dimer from the serum proteins and then analyzing for the presence of the peptide monomer or dimer of interest using LC-MS.

In some embodiments, a peptide monomer or dimer molecule of the present invention exhibits improved solubility or improved aggregation characteristics as compared to a control peptide. Solubility may be determined via any suitable method known in the art. In some embodiments, suitable methods known in the art for determining solubility include incubating peptides in various buffers (Acetate pH4.0, Acetate pH5.0, Phos/Citrate pH5.0, Phos Citrate pH6.0, Phos pH 6.0, Phos pH 7.0, Phos pH7.5, Strong PBS pH 7.5, Tris pH7.5, Tris pH 8.0, Glycine pH 9.0, Water, Acetic acid (pH 5.0 and other known in the art) and testing for aggregation or solubility using standard techniques. These include, but are not limited to, visual precipitation, dynamic light scattering, Circular Dichroism and fluorescent dyes to measure surface hydrophobicity, and detect aggregation or fibrillation, for example. In some embodiments, improved solubility means the peptide monomer or dimer is more soluble in a given liquid than is a control peptide.

In some embodiments, the peptide monomer and dimer molecules of the present invention have less degradation (i.e., more degradation stability), e.g., greater than or about 10% less, greater than or about 20% less, greater than or about 30% less, greater than or about 40 less, or greater than or about 50% less degradation than a control peptide. In some embodiments, degradation stability is determined via any suitable method known in the art. In some embodiments, suitable methods known in the art for determining degradation stability include the method described in Hawe et al J Pharm Sci, VOL. 101, NO. 3, 2012, p 895-913, incorporated herein in its entirety. Such methods are in some embodiments used to select potent peptide monomer or dimer molecules with enhanced shelf lifes.

In some embodiments, peptide dimer or monomer molecules of the present invention have increased redox stability as compared to a control peptide. Methods of determining redox stability are described herein.

In certain embodiments, peptide dimer or monomer molecules of the present invention inhibit or reduce α4β7-mediated inflammation. In related embodiments, peptide monomers or dimers of the present invention inhibit or reduce α4β7-mediated secretion of one or more cytokines. Methods of determining inhibition of cytokine secretion and inhibition of signaling molecules are known in the art.

In certain embodiments, peptide monomer or dimer molecules of the present invention demonstrate increased binding selectivity. In certain instances, peptide monomers or dimers of the present invention binds to α4β7 with at least a two-fold, three-fold, five-fold, or ten-fold greater affinity than the monomers or dimers bind to α4β1.

The peptide monomer or dimer molecules of the present invention demonstrate increased potency as a result of substituting various natural amino acyl residues with N-methylated analog residues. In particular embodiments, potency is measured as IC50 of binding to α4β7, e.g., determined as described herein, while in some embodiments, potency indicates functional activity, e.g., according to a cell adhesion assay as described herein or a PBMC assay described herein. For example, SEQ ID NOs.: 1-32 represent peptide monomer or subunit sequences that are substituted with N(alpha)methylated arginine.

In particular embodiments, any of these superior characteristics of the peptides of the present invention are measured as compared to a control peptide, e.g., a peptide shown in Table 8.

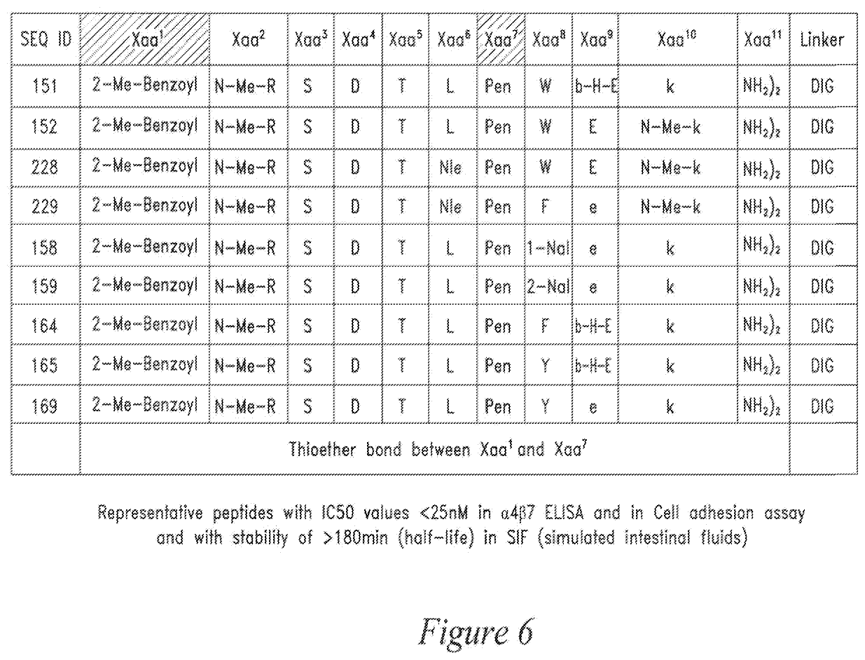

Referring now to FIG. 6 and Tables 5 and 7, charts are provided which include various data illustrating increased potency and/or stability for various non-limiting sample thioether peptide dimer molecules in accordance with the instant invention. Simulated Intestinal Fluid (SIF) Stability assays were performed for the majority of the dimer molecules. A selective sampling of these results is provided in FIG. 6. Indicated thioether peptides in FIG. 6 represent a non-limiting, representative group of dimer peptides with stability of greater than 180 minutes (half-life) in SIF. These thioether dimer compounds further represent IC50 values of less than 25 nM in ELISA as well as cell adhesion assays, further demonstrating their high selectivity for α4β7. For other peptides in FIG. 6, it is expected that they will have an IC50<50 nM in α4β7 ELISA or cell adhersion assays.

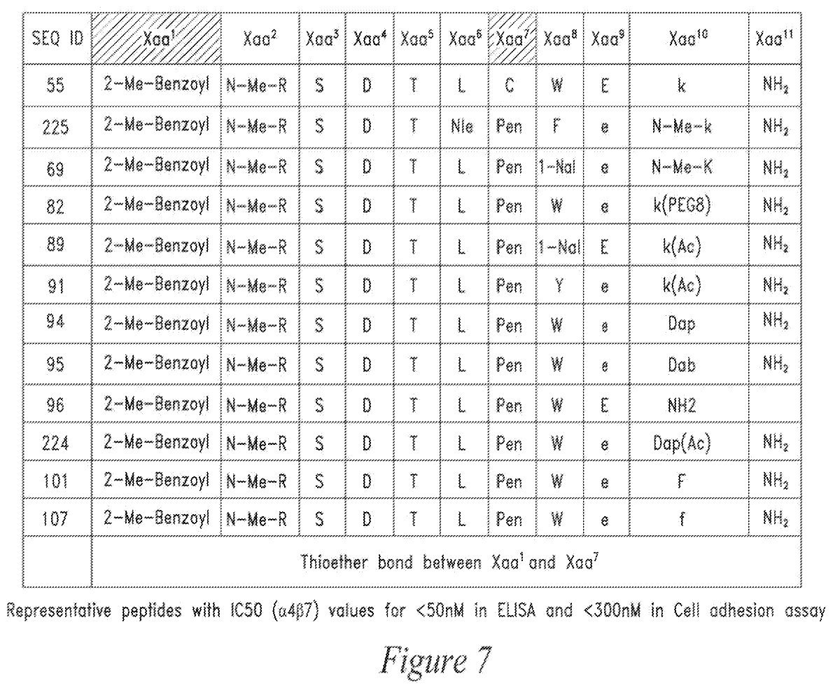

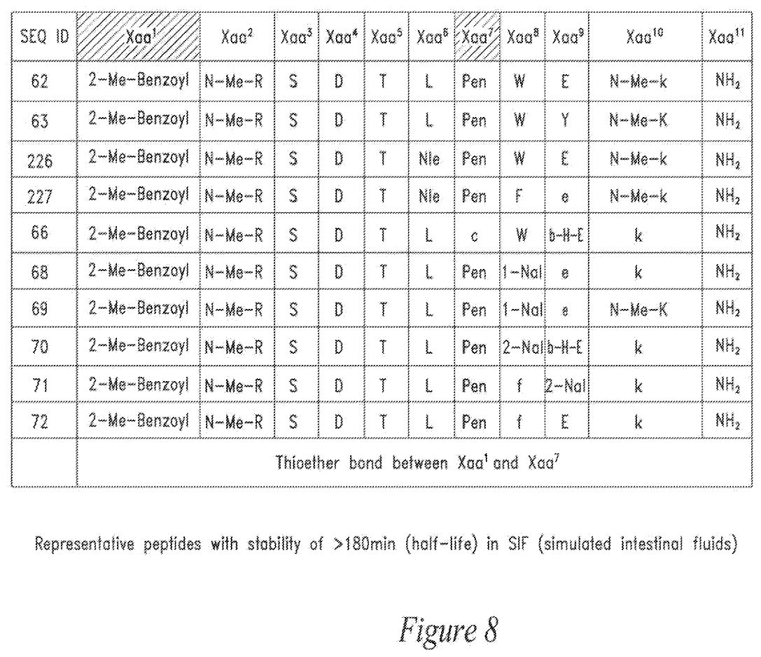

Referring now to FIGS. 7 and 8 and Tables 4 and 6, charts are provided which includes various data illustrating increased potency for various non-limiting illustrative thioether peptide monomers in accordance with the instant invention. Potency assays were performed for all peptide molecules represented by SEQ ID NOs: 22 and 23 and additional peptides as shown. Selectivity assays (for α4β1) were performed for certain thioether peptides. A selective sampling of these results is provided in FIGS. 7 and 8. Improvements in potency for α4β7 were tested in both ELISA and cell adhesion assays.

According to the protocols discussed herein, applicant successfully synthesized and purified all of the integrin antagonist thioether peptides (e.g. peptide monomers and peptide dimers) represented by SEQ ID NOs: 22 to 24 and additional peptides shown in Tables 4-7 and FIGS. 6-8. The majority of these molecules were subjected to an α4β7-MAdCAM Competition ELISA assay, an α4β1-VCAM Competition ELISA assay, and an α4β7-MadCAM cell adhesion assay. Results are provided in Tables 6-7 and FIGS. 6-8. The thioether peptides shown in FIG. 7 represent a non-limiting, representative group of peptides with IC50 values less than 50 nM in ELISA assays. The peptides further represent IC50 values of less than 300 nM in cell adhesion assays. For other peptides with data not shown, it is expected that they will have an IC50<50 nM in α4β7 ELISA or cell adhersion assays.

When Arg is replaced with N-Me-Arg, a significant improvement in potency for α4β7 was shown in both ELISA and cell adhesion assays. N(alpha)methylation further demonstrated increased molecular stability. One having skill in the art will appreciate that methylated isosteres of arginine may further demonstrate similar increases in potency and/or stability.

Referring now to FIGS. 6 and 8 charts are provided which include data illustrating increased stability for various non-limiting sample thioether peptide molecules in accordance with the instant invention. Simulated Intestinal Fluid (SIF) Stability assays were performed for the majority of the peptide molecules. A selective sampling of these results is provided in FIGS. 6 and 8. The thioether peptides in FIGS. 6 and 8 represent a non-limiting, representative group of peptides with stability of greater than 180 minutes (half-life) in SIF.

### Methods of Manufacture and Enhancing Peptide Stability

The peptides (e.g. peptide monomers or peptide dimers) of the present invention may be synthesized by techniques that are known to those skilled in the art. Such techniques include the use of commercially available robotic protein synthesizers (e.g. Symphony multiplex peptide synthesizer from Protein Technologies). In some embodiments, novel peptide monomers or dimer subunits are synthesized and purified using techniques described herein.

Certain aspects of the present invention contemplate peptides comprising thioether bonds. Thioether bonds are cyclized covalent bonds formed between an upstream amino acid or aromatic acid group and a downstream sulfur-containing amino acid or isostere thereof. Thioether bonds of the present invention may be generated using standard techniques in the art, including those described herein. Particular aspects contemplate that the generation of a thioether bond increases gastrointestinal stability of a peptide molecule. Thus, in particular embodiments, gastrointestinal stability of a peptide can be increased by cyclizing the peptide via a thioether bond.

In some embodiments, monomeric subunits of the present invention may be dimerized to form homomeric or heteromeric dimer peptides through known techniques in the art. In certain embodiments, peptide subunits described herein are joined by linker moieties (e.g. linkers shown in Table 3) conjugated at the N or C-termini. A linker may be conjugated to peptide subunit at a C- or N-terminal free amine through techniques known in the art, including but not limited to techniques described herein. Some embodiments contemplate that dimerization of the peptide molecule increases stability, potency, and/or specificity as compared to non-dimerized monomeric subunits of the peptide.

Certain aspects of the present invention contemplate amino acid substitutions that increase stability of a peptide monomer or peptide dimer in different contexts. Accordingly, in certain embodiments, the present invention includes modifying a peptide molecule, e.g., a peptide molecule described herein or Substitutions may be performed by standard techniques known to those of skill in the art. In some embodiments, stability of a peptide (e.g. a peptide monomer or dimer described herein or in Dubree, et al., Selective α4β7 Integrin Antagonist and Their Potential as Anti-inflammatory Agents, J. Med. Chem. 2002, 45, 3451-3457) in simulated intestinal fluids (SIF) is increased by substituting N-Me-Arg for one or more unmethylated arginine residues. In particular embodiments, SIF or gastrointestinal stability of a peptide is increased by substituting Pen for one or more cysteine residues. Certain aspects of the present invention contemplate amino acid substitutions that increase redox stability (i.e. increasing the resistance of a peptide to a change in its oxidation state) of a peptide monomer or peptide dimer described herein. In particular embodiments, redox stability is determined by an assay described herein. In particular embodiments, redox stability is increased by at least 20%, at least 50%, at least 2-fold, at least 3-fold, at least 4-fold, at least 5-fold, or at least 10-fold as compared to a control peptide. Substitutions may be performed by standard techniques known to those of skill in the art. In some embodiments, redox or gastrointestinal stability of a peptide (e.g. peptide monomer or dimer described herein) is increased by substituting N-Me-Arg for one or more unmethylated arginine residues.

In particular embodiments, the invention provides a method for stabilizing a peptide molecule, e.g., a peptide molecule described herein, comprising cyclizing the peptide molecule by forming a thioether bond between Xaa⁴ and Xaa¹⁰.

In certain embodiments, the invention includes a method for stabilizing a peptide molecule, e.g., of Formula (II), comprising: substituting Xaa¹ with an aromatic acid group capable of forming a thioether bond with Xaa⁷; substituting Xaa⁷ with an amino acid residue that is capable of forming a thioether bond with Xaa¹; and forming a thioether bond between Xaa¹ and Xaa⁷ to provide a cyclized peptide. In certain embodiments, Xaa⁷ is selected from the group consisting of Cys, N-Me-Cys, D-Cys, HCys, Pen, and D-Pen. In certain embodiments, Xaa¹ is a 2-methylbenzoyl moiety. The same method applies to peptide molecules, e.g., of Formula (I), where Xaa⁴ and Xaa¹⁰ are substituted and cyclized instead of Xaa¹ and Xaa⁷, respectively.

### Methods of Treatment and Pharmaceutical Compositions

In some embodiments, the present invention provides a method for treating an individual or subject afflicted with a condition or indication characterized by integrin binding, wherein the method comprises providing or administering to the individual or subject an integrin antagonist thioether peptide molecule described herein, e.g., as represented by SEQ ID NOs: 1-384 or shown in Tables 5-7. In particular embodiments, the individual or subject is provided with or administered with a pharmaceutical composition comprising the peptide monomer or peptide dimer of the invention. In particular embodiments, subjects or individuals are mammals, e.g., humans or non-human mammals, such as a dog, cat or horse.

In one embodiment, a method is provided for treating an individual or subject afflicted with a condition or indication characterized by inappropriate trafficking of cells expressing α4β7 to tissues comprising cells expressing MAdCAM, comprising administering to the individual or subject an α4β7-antagonist peptide molecule described herein, e.g., SEQ ID NOs: 1-384 or Tables 4 and 5, in an amount sufficient to inhibit (partially or fully) the trafficking of cells expressing α4β7 to tissues comprising cells expressing MAdCAM.

In a further related embodiments, the present invention includes a method for treating a condition in a subject or individual in need thereof, wherein the condition is treatable by reducing the activity (partially or fully) of α4β7 in the subject, comprising providing or administering an α4β7-antagonist peptide molecule described herein to the subject. In particular embodiments, the condition is an inflammatory condition of the gastrointestinal system.

In a further related embodiments, the present invention includes a method for treating a subject, e.g., a mammal or human, afflicted with a condition that is associated with a biological function α4β7, comprising providing or administering to the subject a thioether peptide molecule described herein, e.g., a peptide monomer or peptide dimer having a structure of Formula (I) or (II), in an amount sufficient to inhibit (partially or fully) the biological function of α4β7 to tissues expressing MAdCAM. In particular embodiments, the subject is provided with an effective amount of the peptide monomer or peptide dimer sufficient to at least partially inhibit the biological function of α4β7 to tissues expressing MAdCAM. In certain embodiments, the condition is inflammatory bowel disease. In additional embodiments, the invention includes a method of treating or preventing a disease or condition in a subject in need thereof, comprising providing or administering to the subject, e.g., a mammal, an effective amount of a peptide dimer or peptide monomer described herein, wherein the disease or condition is selected from the group consisting of Inflammatory Bowel Disease (IBD) (including adult IBD, pediatric IBD and adolescent IBD), ulcerative colitis, Crohn's disease, Celiac disease (nontropical Sprue), enteropathy associated with seronegative arthropathies, microscopic colitis, collagenous colitis, eosinophilic gastroenteritis, radiotherapy, chemotherapy, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, primary sclerosing cholangitis, human immunodeficiency virus (HIV) infection in the GI tract, eosinophilic asthma, eosinophilic esophagitis, gastritis, colitis, microscopic colitis and graft versus host disease (GVDH) (including intestinal GVDH). In particular embodiments of any of the methods of treatment described herein, the subject has been diagnosed with or is considered to be at risk of developing one of these diseases or conditions.

In particular embodiments of any of the methods of treatment described herein, the peptide molecule (or pharmaceutical composition comprising the peptide molecule) is administered to the individual by a form of administration selected from the group consisting of oral, intravenous, peritoneal, intradermal, subcutaneous, intramuscular, intrathecal, inhalation, vaporization, nebulization, sublingual, buccal, parenteral, rectal, vaginal, and topical.

In certain embodiments, the α4β7 integrin antagonist peptide molecule comprises an increased half-life as compared to other peptides. In particular embodiments, the increased half-life is at least one day in vitro or in vivo. In certain embodiments wherein the increased half-life is equal to or greater than a period consistent with no more frequent than twice daily dosing in vivo, the α4β7 integrin antagonist peptide molecule is provided in a pharmaceutical preparation that is administered orally. In certain embodiments wherein the increased half-life is from approximately 12 hours to greater than 24 in vivo, the α4β7 integrin antagonist peptide molecule is provided in a pharmaceutical preparation that is administered parenterally. In certain embodiments when the increased half-life is from approximately 12 hours to greater than 24 hours in vivo, the α4β7 integrin antagonist peptide molecule is provided in a pharmaceutical preparation that is administered topically.

In some embodiments, the present invention provides a method whereby a pharmaceutical composition comprising an integrin antagonist thioether peptide molecule described herein, e.g., SEQ ID NOs: 1-384 or Tables 4 or 5, is administered to a subject or patient as a first treatment. In another embodiment, the method further comprises administering to the subject a second treatment, i.e., a second active agent. In another embodiment, the second treatment or active agent is administered to the subject before and/or simultaneously with and/or after the pharmaceutical composition is administered to the subject. In other embodiment, the second treatment or active agent comprises an anti-inflammatory agent. In another embodiment, the second treatment or active agent (which may be present in a pharmaceutical composition) comprises an agent selected from the group consisting of non-steroidal anti-inflammatory drugs, steroids, and immune modulating agents. In another embodiment, the method comprises administering to the subject a third treatment.

The thioether peptide monomer and dimer molecules of the invention, including but not limited to those specified in the examples, possess integrin-antagonist activity. In certain embodiments, peptide integrin inhibitors (e.g. thioether peptide monomers and dimers described herein) are administered to a subject in need of treatment for Inflammatory Bowel Disease (IBD), ulcerative colitis, Crohn's disease, Celiac disease (nontropical Sprue), enteropathy associated with seronegative arthropathies, microscopic or collagenous colitis, eosinophilic gastroenteritis, radio- and chemotherapy, or pouchitis resulting after proctocolectomy and ileoanal anastomosis and various forms of gastrointestinal cancer, osteoporosis, arthritis, multiple sclerosis, chronic pain, weight gain, and/or depression.

In another embodiment, peptide integrin inhibitors of the present invention are administered to a subject in need of treatment for pancreatitis, insulin-dependent diabetes mellitus, mastitis, cholecystitis, cholangitis, pericholangitis, chronic bronchitis, chronic sinusitis, asthma and/or graft versus host disease. In addition, these peptide monomer and dimer molecules may be useful in the prevention or reversal of these diseases when used in combination with currently available therapies, medical procedures, and therapeutic agents.

In one embodiment, a method is provided for treating an individual or subject afflicted with a condition or indication characterized by α4β7 integrin binding, wherein the method comprises administering to the individual or subject an effective amount of an α4β7 integrin antagonist peptide molecule described herein, e.g., SEQ ID NOs: 1-384 or Tables 4 or 5. In some instances, an α4β7 integrin antagonist peptide molecule described herein, e.g., corresponding to SEQ ID NOs: 1-384 or Tables 4 or 5, and having high specificity for α4β7 is administered to an individual as part of a therapeutic treatment for a condition or indication characterized by α4β7 integrin binding.

In particular embodiments, the peptide molecules of the present invention are present in a pharmaceutical composition further comprising one or more pharmaceutically acceptable diluents, carriers, or excipients. In particular embodiments, they are formulated as a liquid or solid. In particular embodiments, they are formulated as a tablet or capsule, or as a liquid suspension. Some embodiments of the present invention further provide a method for treating an individual with an α4β7 integrin antagonist peptide molecule of the present invention that is suspended in a sustained-release matrix. A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. On particular biodegradable matrix is a matrix of one of either polylactide, polyglycolide, or polylactide co-glycolide (co-polymers of lactic acid and glycolic acid).

In some aspects, the invention provides a pharmaceutical composition for oral delivery. The various embodiments and thioether peptide molecule compositions of the instant invention may be prepared for oral administration according to any of the methods, techniques, and/or delivery vehicles described herein. Further, one having skill in the art will appreciate that the peptide molecule compositions of the instant invention may be modified or integrated into a system or delivery vehicle that is not disclosed herein, yet is well known in the art and compatible for use in oral delivery of small peptide molecules.

Oral dosage forms or unit doses compatible for use with the peptides of the present invention may include a mixture of peptide active drug components, and nondrug components or excipients, as well as other non-reusable materials that may be considered either as an ingredient or packaging. Oral compositions may include at least one of a liquid, a solid, and a semi-solid dosage forms. In some embodiments, an oral dosage form is provided comprising an effective amount of a thioether peptide molecule described herein, e.g., corresponding to any of SEQ ID NOs: 1-384 or Tables 4 or 5, wherein the dosage form comprises at least one of a pill, a tablet, a capsule, a gel, a paste, a drink, and a syrup. In some instances, an oral dosage form is provided that is designed and configured to achieve delayed release of the thioether peptide molecule in the small intestine of the subject.

In one embodiment, an oral pharmaceutical composition comprising a thioether peptide of the present invention comprises an enteric coating that is designed to delay release of the peptide molecule in the small intestine. In at least some embodiments, a pharmaceutical composition is provided which comprises a peptide molecule described herein, e.g., corresponding to any of SEQ ID NOs: 1-384, or Tables 4 or 5, and a protease inhibitor, such as aprotinin, in a delayed release pharmaceutical formulation. In some instances it is preferred that a pharmaceutical composition of the instant invention comprise an enteric coat that is soluble in gastric juice at a pH of about 5.0 or higher. In at least one embodiment, a pharmaceutical composition is provided comprising an enteric coating comprising a polymer having dissociable carboxylic groups, such as derivatives of cellulose, including hydroxypropylmethyl cellulose phthalate, cellulose acetate phthalate and cellulose acetate trimellitate and similar derivatives of cellulose and other carbohydrate polymers.

In one embodiment, a pharmaceutical composition comprising a thioether peptide molecule described herein, e.g., corresponding to any of SEQ ID NOs: 1-384 or Tables 4 and 5, is provided in an enteric coating, the enteric coating being designed to protect and release the pharmaceutical composition in a controlled manner within the lower gastrointestinal system of a subject, and to avoid systemic side effects. In addition to enteric coatings, the peptide molecules of the instant invention may be encapsulated, coated, engaged or otherwise associated within any compatible oral drug delivery system or component. For example, in some embodiments a peptide molecule of the present invention is provided in a lipid carrier system comprising at least one of polymeric hydrogels, nanoparticles, microspheres, micelles, and other lipid systems.

To overcome peptide degradation in the small intestine, some implementations of the present invention comprise a hydrogel polymer carrier system in which a peptide molecule in accordance with the present invention is contained, whereby the hydrogel polymer protect the peptide from proteolysis in the small intestine. The peptide molecules of the present invention may further be formulated for compatible use with a carrier system that is designed to increase the dissolution kinetics and enhance intestinal absorption of the peptides. These methods include the use of liposomes, micelles and nanoparticles to increase GI tract permeation of peptides.

Various bioresponsive systems may also be combined with one or more thioether peptide molecules of the present invention to provide a pharmaceutical agent for oral delivery. In some embodiments, a peptide molecule of the instant invention is used in combination with a bioresponsive system, such as hydrogels and mucoadhesive polymers with hydrogen bonding groups (e.g., PEG, poly(methacrylic) acid [PMAA], cellulose, Eudragit®, chitosan and alginate) to provide a therapeutic agent for oral administration. Other embodiments include a method for optimizing or prolonging drug residence time for a peptide molecule disclosed herein, wherein the surface of the peptide molecule is modified to comprise mucoadhesive properties through hydrogen bonds, polymers with linked mucins or/and hydrophobic interactions. These modified peptide molecules may demonstrate increase drug residence time within the subject, in accordance with a desired feature of the invention. Moreover, targeted mucoadhesive systems may specifically bind to receptors at the enterocytes and M-cell surfaces, thereby further increasing the uptake of particles containing the peptide molecules.

Other embodiments comprise a method for oral delivery of a thioether peptide molecule described herein, e.g., corresponding to any of SEQ ID NOs: 1-384 or Tables 4 or 5, wherein the peptide molecule is used in combination with permeation enhancers that promote the transport of the peptides across the intestinal mucosa by increasing paracellular or transcellular permeation. For example, in one embodiment a permeation enhancer is combined with a thioether peptide molecule described herein, e.g., corresponding to any of SEQ ID NOs: 1-384, or Tables 4 or 5, wherein the permeation enhancer comprises at least one of a long-chain fatty acid, a bile salt, an amphiphilic surfactant, and a chelating agent. In one embodiment, a permeation enhancer comprising sodium N-[(hydroxybenzoyl)amino]caprylate is used to form a weak noncovalent association with the peptide molecule of the instant invention, wherein the permeation enhancer favors membrane transport and further dissociation once reaching the blood circulation. In another embodiment, a peptide molecule of the present invention is conjugated to oligoarginine, thereby increasing cellular penetration of the peptide into various cell types. Further, in at least one embodiment a noncovalent bond is provided between a thioether peptide molecule described herein, e.g., SEQ ID NOs: 1-384 or Tables 4 or 5, and a permeation enhancer selected from the group consisting of a cyclodextrin (CD) and a dendrimers, wherein the permeation enhancer reduces peptide aggregation and increasing stability and solubility for the peptide molecule.

Other embodiments of the invention provide a method for treating an individual with an α4β7 integrin antagonist thioether peptide molecule having an increased half-life. In one aspect, the present invention provides an integrin antagonist thioether peptide molecule having a half-life of at least several hours to one day in vitro or in vivo (e.g., when administered to a human subject) sufficient for daily (q.d.) or twice daily (b.i.d.) dosing of a therapeutically effective amount. In another embodiment, the peptide molecule has a half-life of three days or longer sufficient for weekly (q.w.) dosing of a therapeutically effective amount. Further, in another embodiment the peptide molecule has a half-life of eight days or longer sufficient for bi-weekly (b.i.w.) or monthly dosing of a therapeutically effective amount. In another embodiment, the thioether peptide molecule is derivatized or modified such that is has a longer half-life as compared to an underivatized or unmodified peptide molecule. In another embodiment, the peptide molecule contains one or more chemical modifications to increase serum half-life.

When used in at least one of the treatments or delivery systems described herein, a therapeutically effective amount of one of the thioether peptide molecules of the present invention may be employed in pure form or, where such forms exist, in pharmaceutically acceptable salt form. As used herein, a “therapeutically effective amount” of the compound of the invention is meant to describe a sufficient amount of the thioether peptide molecule to treat an integrin-related disease, (for example, to reduce inflammation associated with IBD) at a desired benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific compound employed; e) the duration of the treatment; f) drugs used in combination or coincidental with the specific compound employed, and like factors well known in the medical arts. For example, it is well within the skill of the art to start doses of the compound at levels lower than those required to achieve the desired therapeutic effect and to gradually increase the dosage until the desired effect is achieved.

Alternatively, a compound of the present invention may be administered as pharmaceutical compositions containing the thioether peptide molecule of interest in combination with one or more pharmaceutically acceptable excipients. A pharmaceutically acceptable carrier or excipient refers to a non-toxic solid, semi-solid or liquid filler, diluent, encapsulating material or formulation auxiliary of any type. The compositions may be administered parenterally, intracisternally, intravaginally, intraperitoneally, intrarectally, topically (as by powders, ointments, drops, suppository, or transdermal patch), rectally, or buccally. The term “parenteral” as used herein refers to modes of administration which include intravenous, intramuscular, intraperitoneal, intrasternal, subcutaneous, intradermal and intraarticular injection and infusion.

In particular embodiments, pharmaceutical compositions for parenteral injection comprise pharmaceutically acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

Injectable depot forms are made by forming microencapsule matrices of the drug in biodegradable polymers such as polylactide-polyglycolide, poly(orthoesters), poly(anhydrides), and (poly)glycols, such as PEG. Depending upon the ratio of drug to polymer and the nature of the particular polymer employed, the rate of drug release can be controlled. Depot injectable formulations are also prepared by entrapping the drug in liposomes or microemulsions which are compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Topical administration includes administration to the skin or mucosa, including surfaces of the lung and eye. Compositions for topical lung administration, including those for inhalation and intranasal, may involve solutions and suspensions in aqueous and non-aqueous formulations and can be prepared as a dry powder which may be pressurized or non-pressurized. In non-pressurized powder compositions, the active ingredient in finely divided form may be used in admixture with a larger-sized pharmaceutically acceptable inert carrier comprising particles having a size, for example, of up to 100 micrometers in diameter. Suitable inert carriers include sugars such as lactose.

Alternatively, the composition may be pressurized and contain a compressed gas, such as nitrogen or a liquefied gas propellant. The liquefied propellant medium and indeed the total composition is preferably such that the active ingredient does not dissolve therein to any substantial extent. The pressurized composition may also contain a surface active agent, such as a liquid or solid non-ionic surface active agent or may be a solid anionic surface active agent. It is preferred to use the solid anionic surface active agent in the form of a sodium salt.

A further form of topical administration is to the eye. A compound of the invention is delivered in a pharmaceutically acceptable ophthalmic vehicle, such that the compound is maintained in contact with the ocular surface for a sufficient time period to allow the compound to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically acceptable ophthalmic vehicle may, for example, be an ointment, vegetable oil or an encapsulating material. Alternatively, the compounds of the invention may be injected directly into the vitreous and aqueous humour.

Compositions for rectal or vaginal administration are preferably suppositories which may be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art.

Total daily dose of the compositions of the invention to be administered to a human or other mammal host in single or divided doses may be in amounts, for example, from 0.0001 to 300 mg/kg body weight daily and more usually 1 to 300 mg/kg body weight.

### Non-Invasive Detection of Intestinal Inflammation

The thioether peptides of the invention may be used for detection, assessment and diagnosis of intestinal inflammation by microPET imaging using an orally stable thioether peptide monomer or dimer molecule selected from and corresponding to SEQ ID NOs: 1-32, or described herein or in the accompanying Figures, and that is further labeled with at least one of a chelating group and a detectable label as part of a non-invasive diagnostic procedure. In one embodiment, an integrin antagonist thioether peptide monomer or dimer molecule is conjugated with a bifunctional chelator to provide an orally stable peptide molecule. In another embodiment, an integrin antagonist peptide monomer or dimer molecule is radiolabeled to provide an orally stable peptide molecule. The orally stable, chelated or radiolabeled peptide molecule is then administered to a subject orally or rectally. In one embodiment, the orally stable peptide monomer or dimer molecule is included in drinking water. Following uptake of the peptide molecules, microPET imaging may be used to visualize inflammation throughout the subject's bowels and digestive track.

### EXAMPLES

### Example 1

### Synthesis of Thioether Peptide Monomer and Dimer Molecules

The peptide monomers or peptide subunits of the present invention may be synthesized by many techniques that are known to those skilled in the art. Novel and unique thioether peptide molecules were synthesized and purified, and dimerized in the case of peptide dimer molecules, using the techniques provided herein.

### Synthesis

The peptides of the present invention were synthesized using the Merrifield solid phase synthesis techniques on Protein Technology's Symphony multiple channel synthesizer. The peptides were assembled using HBTU (O-Benzotriazole-N,N,N′,N′-tetramethyl-uronium-hexafluoro-phosphate), Diisopropylethylamine(DIEA) coupling conditions. Rink Amide MBHA resin (100-200 mesh, 0.57 mmol/g) was used for peptides with C-terminal amides and pre-loaded Wang Resin with N-a-Fmoc protected amino acid was used for peptides with C-terminal acids. The coupling reagents (HBTU and DIEA premixed) were prepared at 100 mmol concentration. Similarly amino acids solutions were prepared at 100 mmol concentration. The peptides were assembled using standard Symphony protocols.

### Assembly

The peptide sequences were assembled as follows: Resin (250 mg, 0.14 mmol) in each reaction vial was washed twice with 4 ml of DMF followed by treatment with 2.5 ml of 20% 4-methyl piperidine (Fmoc de-protection) for 10 min. The resin was then filtered and washed two times with DMF (4 ml) and re-treated with N-methyl piperidine for additional 30 minute. The resin was again washed three times with DMF (4 ml) followed by addition 2.5 ml of amino acid and 2.5 ml of HBTU-DIEA mixture. After 45 min of frequent agitations, the resin was filtered and washed three timed with DMF (4 ml each). For a typical peptide of the present invention, double couplings were performed. For N-Me-Arg and 2-(Chloromethyl)benzoic acid coupling, double coupling of 2.0 eq 2-(Chloromethyl)benzoic acid, 2.0 eq PyAOP, and 4 eq DIEA in DMF for 1 hr. Reaction completion was monitored using the Chloranil test. After completing the coupling reaction, the resin was washed three times with DMF (4 ml each) before proceeding to the next amino acid coupling.

### Cleavage

Following completion of the peptide assembly, the peptide was cleaved from the resin by treatment with cleavage reagent, TFA:water:TIPS (92.5 v:5 v:2.5 v). The cleavage reagent was able to successfully cleave the peptide from the resin, as well as all remaining side chain protecting groups.

The cleavage reaction mixture was stirred for 2 h at room temperature. The spent resin was filtered off. The filtrate was then precipitated into cold ethyl ether and centrifuged to collect the peptide. The ethyl ether was decanted, and the solid precipitate was washed two times with cold ethyl ether. The crude peptide was dissolved in a solution of acetonitrile:water (7:3 with 1% TFA) and filtered. The quality of linear peptide was then verified using electrospray ionization mass spectrometry (ESI-MS) (Micromass/Waters ZQ) before being purified.

### Thioether Bond Formation

The unpurified linear monomer (50 mg) was dissolved in 50:50 ACN:water (2.5 mg/ml) then diluted to about 1 mg/mL in 0.1M Tris-HCl pH8.5 buffer. The reaction was monitored using LCMS. When the reaction is completed (usually overnight), diluted the reaction mixture with water and purify by RP-HPLC.

### Purification

Analytical reverse-phase, high performance liquid chromatography (HPLC) was performed on a Gemini C18 column (4.6 mm×250 mm) (Phenomenex). Semi-Preparative reverse phase HPLC was performed on a Gemini 10 μm C18 column (22 mm×250 mm) (Phenomenex) or Jupiter 10 μm, 300 A° C18 column (21.2 mm×250 mm) (Phenomenex). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 15 mL/min (preparative). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 15 mL/min (preparative).

### Linker Activation and Dimerization

Small Scale DIG Linker Activation Procedure: 5 mL of NMP was added to a glass vial containing IDA diacid (304.2 mg, 1 mmol), N-hydroxysuccinimide (NHS, 253.2 mg, 2.2 eq. 2.2 mmol) and a stirring bar. The mixture was stirred at room temperature to completely dissolve the solid starting materials. N,N′-Dicyclohexylcarbodiimide (DCC, 453.9 mg, 2.2 eq., 2.2 mmol) was then added to the mixture. Precipitation appeared within 10 min and the reaction mixture was further stirred at room temperature overnight. The reaction mixture was then filtered to remove the precipitated dicyclohexylurea (DCU). The activated linker was kept in a closed vial prior to use for dimerization. The nominal concentration of the activated linker was approximately 0.20 M.

For dimerization using PEG linkers, there was no pre-activation step involved. Commercially available pre-activated bi-functional PEG linkers were used.

Dimerization Procedure: 2 mL of anhydrous DMF was added to a vial containing peptide monomer (0.1 mmol). The pH of the peptide was then adjusted to 8-9 with DIEA. Activated linker (IDA or PEG13, PEG 25) (0.48 eq relative to monomer, 0.048 mmol) was then added to the monomer solution. The reaction mixture was stirred at room temperature for one hour. Completion of the dimerization reaction was monitored using analytical HPLC. The time for completion of dimerization reaction varied depending upon the linker. After completion of reaction, the peptide was precipitated in cold ether and centrifuged. The supernatant ether layer was discarded. The precipitation step was repeated twice. The crude dimer was then purified using reverse phase HPLC (Luna C18 support, 10 u, 100A, Mobile phase A: water containing 0.1% TFA, mobile phase B: Acetonitrile (ACN) containing 0.1% TFA, gradient of 15% B and change to 45% B over 60 min, flow rate 15 ml/min). Fractions containing pure product were then freeze-dried on a lyophilizer.

The peptide monomers and peptide dimers shown in Tables 4 and 5 were synthesized and further characterized. Table 4 shows various monomer peptide compounds according to various non-limiting representative embodiments of the present invention. The amino acid residues are numbers Xaa¹⁻¹⁰, in accordance with Formula (II). However, these residues should be understood to also correspond to Xaa⁴⁻¹³ in Formula (I). The amino acid sequence of the peptide is shown, wherein “2-benzyl” indicates 2-methylbenzoyl, and lower case letters indicate D-amino acids. Each peptide is cyclized via an intramolecular thioether bond between the amino acid residue or moiety shown at position 1 and the amino acid residue shown at position 7. Table 5 shows various peptide dimer compounds according to various non-limiting representative embodiments of the present invention. The amino acid sequence of the peptide is shown, wherein “2-benzyl” indicates 2-methylbenzoyl, and lower case letters indicate D-amino acids. The amino acid residues are numbers Xaa¹⁻¹⁰, in accordance with Formula (II). However, these residues should be understood to also correspond to Xaa⁴⁻¹³ in Formula (I). Each monomer subunit of the peptide dimer is cyclized via an intramolecular thioether bond between the amino acid residue or moiety shown at position 1 and the amino acid residue shown at position 7. The peptide monomer subunits of the peptide dimers are dimerized at their C-termini by the indicated DIG, ADA, IDA, IDA-Palm, IDA-Lauryl, IDA-oleoyl, or IDA-PEG linker.

TABLE 4
Illustrative Thioether Monomers

| SEQ | Peptide | | | | | | | | | | | |
|---|---|---|---|---|---|---|---|---|---|---|---|---|
| ID NO | sequence | 1 | 2 | 3 | 4 | 5 | 6 | 7 | 8 | 9 | 10 | |
| 391 | (thioether) | Acetyl | N—Me—R | S | D | T | L | C | W | k | NH2 | |
| 392 | (thioether) | Acetyl | N—Me—R | S | D | T | L | homoCys | W | k | NH2 | |
| 51 | (thioether) | Propionyl | N—Me—R | S | D | T | L | C | W | k | NH2 | |
| 52 | (thioether) | alpha- | N—Me—R | S | D | T | L | C | W | k | NH2 | |
| | | bromoispbutyryl | | | | | | | | | | |
| 53 | (thioether) | Acetyl | N—Me—R | S | D | T | L | Pen | W | k | NH2 | |
| 54 | (thioether) | Propionyl | N—Me—R | S | D | T | L | Pen | W | k | NH2 | |
| 55 | (thioether) | 2-Benzyl | N—Me—R | S | D | T | L | C | W | E | k | NH2 |
| 56 | (thioether) | 2-Benzyl | N—Me—R | S | D | T | L | Pen | W | E | k | NH2 |
| 57 | (thioether) | Propionyl | N—Me—R | S | D | T | L | hC | W | k | NH2 | |
| 58 | ((thioether) | Butyryl | N—Me—R | S | D | T | L | C | W | k | NH2)2 | |
| 59 | (thioether) | 2-Benzyl | R | S | D | T | L | C | W | k | NH2 | |
| 60 | (thioether) | 2-Benzyl | N—Me—R | S | D | T | L | Pen | W | e | k | NH2 |
| 61 | (thioether) | 2-Benzyl | N—Me—R | S | D | T | L | Pen | W | b-H-E | k | NH2 |
| 62 | (thioether) | 2-Benzyl | N—Me—R | S | D | T | L | Pen | W | E | N—Me-k | NH2 |
| 63 | (thioether) | 2-Benzyl | N—Me—R | S | D | T | L | Pen | W | Y | N—Me—K | NH2 |
| 64 | (thioether) | 2-Benzyl | N—Me—R | S | D | T | Nle | Pen | W | E | k | NH2 |
| 65 | (thioether) | 2-Benzyl | N—Me—R | S | D | T | L | Pen | F | e | k | NH2 |
| 66 | (thioether) | 2-Benzyl | N—Me—R | S | D | T | L | c | W | b-H-E | k | NH2 |
| 67 | (thioether) | 2-Benzyl | N—Me—R | S | D | T | L | Hcys | W | E | k | NH2 |
| 68 | (thioether) | 2-Benzyl | N—Me—R | S | D | T | L | Pen | 1-Nal | e | k | NH2 |
| 69 | (thioether) | 2-Benzyl | N—Me—R | S | D | T | L | Pen | 1-Nal | e | N—Me—K | NH2 |
| 70 | (thioether) | 2-Benzyl | N—Me—R | S | D | T | L | Pen | 2-Nal | b-H-E | k | NH2 |
| 71 | (thioether) | 2-Benzyl | N—Me—R | S | D | T | L | Pen | f | 2-Nal | k | NH2 |
| 72 | (thioether) | 2-Benzyl | N—Me—R | S | D | T | L | Pen | f | E | k | NH2 |
| 73 | (thioether) | 2-Benzyl | N—Me—R | S | D | T | L | Pen | F | b-H-E | k | NH2 |
| 74 | (thioether) | 2-Benzyl | N—Me—R | S | D | T | L | Pen | Y | b-H-E | k | NH2 |
| 75 | (thioether) | 2-Benzyl | N—Me—R | S | D | T | L | Pen | 2-Nal | e | k | NH2 |
| 76 | (thioether) | 2-Benzyl | N—Me—R | S | D | T | L | Pen | F(CF3) | E | k | NH2 |
| 77 | (thioether) | 2-Benzyl | N—Me—R | S | D | T | L | Pen | 1Nal | E | k | NH2 |
| 78 | (thioether) | 2-Benzyl | N—Me—R | S | D | T | L | Pen | Y | E | k | NH2 |
| 79 | (thioether) | 2-Benzyl | N—Me—R | S | D | T | L | Pen | Y | e | k | NH2 |
| 80 | (thioether) | 2-Benzyl | N—Me—R | S | D | T | L | Pen | W | E | k(Ac) | NH2 |
| 81 | (thioether) | 2-Benzyl | N—Me—R | S | D | T | L | Pen | W | e | k(Ac) | NH2 |
| 82 | (thioether) | 2-Benzyl | N—Me—R | S | D | T | L | Pen | W | e | k(PEG8) | NH2 |
| 83 | (thioether) | 2-Benzyl | N—Me—R | S | D | T | L | Pen | W | b-H-E | k(Ac) | NH2 |
| 84 | (thioether) | 2-Benzyl | N—Me—R | S | D | T | L | Pen | W | E | N—Me-k(Ac) | NH2 |
| 85 | (thioether) | 2-Benzyl | N—Me—R | S | D | T | L | Pen | W | Y | N—Me—K(Ac) | NH2 |
| 86 | (thioether) | 2-Benzyl | N—Me—R | S | D | T | Nle | Pen | W | E | k(Ac) | NH2 |
| 87 | (thioether) | 2-Benzyl | N—Me—R | S | D | T | L | Pen | F | e | k(Ac) | NH2 |
| 88 | (thioether) | 2-Benzyl | N—Me—R | S | D | T | L | Pen | F(CF3) | E | k(Ac) | NH2 |
| 89 | (thioether) | 2-Benzyl | N—Me—R | S | D | T | L | Pen | 1Nal | E | k(Ac) | NH2 |
| 90 | (thioether) | 2-Benzyl | N—Me—R | S | D | T | L | Pen | Y | E | k(Ac) | NH2 |
| 91 | (thioether) | 2-Benzyl | N—Me—R | S | D | T | L | Pen | Y | e | k(Ac) | NH2 |
| 92 | ((thioether) | 2-Benzyl | N—Me—R | S | D | T | L | Pen | W | E | Dap | NH2 |
| 93 | ((thioether) | 2-Benzyl | N—Me—R | S | D | T | L | Pen | W | E | Dab | NH2 |
| 94 | ((thioether) | 2-Benzyl | N—Me—R | S | D | T | L | Pen | W | e | Dap | NH2 |
| 95 | ((thioether) | 2-Benzyl | N—Me—R | S | D | T | L | Pen | W | e | Dab | NH2 |
| 96 | ((thioether) | 2-Benzyl | N—Me—R | S | D | T | L | Pen | W | E | NH2 | |
| 97 | ((thioether) | 2-Benzyl | N—Me—R | S | D | T | L | Pen | W | e | NH2 | |
| 98 | ((thioether) | 2-Benzyl | N—Me—R | S | D | T | L | Pen | W | e | NH2 | |
| 99 | ((thioether) | 2-Benzyl | N—Me—R | S | D | T | L | Pen | W | e | L | NH2 |
| 100 | ((thioether) | 2-Benzyl | N—Me—R | S | D | T | L | Pen | W | e | S | NH2 |
| 101 | ((thioether) | 2-Benzyl | N—Me—R | S | D | T | L | Pen | W | e | F | NH2 |
| 102 | ((thioether) | 2-Benzyl | N—Me—R | S | D | T | L | Pen | W | e | H | NH2 |
| 103 | ((thioether) | 2-Benzyl | N—Me—R | S | D | T | L | Pen | W | e | Q | NH2 |
| 104 | ((thioether) | 2-Benzyl | N—Me—R | S | D | T | L | Pen | W | e | Y | NH2 |
| 105 | ((thioether) | 2-Benzyl | N—Me—R | S | D | T | L | Pen | W | e | l | NH2 |
| 106 | ((thioether) | 2-Benzyl | N—Me—R | S | D | T | L | Pen | W | e | s | NH2 |
| 107 | ((thioether) | 2-Benzyl | N—Me—R | S | D | T | L | Pen | W | e | f | NH2 |
| 108 | ((thioether) | 2-Benzyl | N—Me—R | S | D | T | L | Pen | W | e | e | NH2 |
| 109 | ((thioether) | 2-Benzyl | N—Me—R | S | D | T | L | Pen | W | e | h | NH2 |
| 110 | ((thioether) | 2-Benzyl | N—Me—R | S | D | T | L | Pen | W | e | y | NH2 |
| 111 | ((thioether) | 3-Benzyl | N—Me—R | S | D | T | L | Pen | W | e | k | NH2 |
| 112 | (thioether) | 4-Benzyl | N—Me—R | S | D | T | L | Pen | W | e | k | NH2 |
| 113 | ((thioether) | 2-Benzyl | N—Me—R | S | D | T | L | Pen | W | e | E | NH2 |
| 114 | ((thioether) | 2-Benzyl | N—Me—R | S | D | T | L | Pen | 2-Nal | e | NH2 | |
| 115 | ((thioether) | 2-Benzyl | N—Me—R | S | D | T | L | Pen | W | E(OMe) | k | NH2 |
| 116 | ((thioether) | 2-Benzyl | N—Me—R | S | D | T | L | Pen | 2-Nal | NH2 | | |
| 117 | (thioether) | 2-Benzyl | N—Me—R | S | D | T | L | C | Tic | E | k | NH2 |
| 118 | (thioether) | 2-Benzyl | N—Me—R | S | D | T | L | C | Tic | k | OH | |
| 119 | ((thioether) | 2-Benzyl | N—Me—R | S | D | T | L | Pen | Atc | bHE | NH2 | |
| 120 | ((thioether) | 2-Benzyl | N—Me—R | S | D | T | L | Pen | erythro-b-F—S | bHE | NH2 | |
| 121 | ((thioether) | 2-Benzyl | N—Me—R | S | D | T | L | Pen | erythro-b-F—S | bHE | NH2 | |
| 122 | ((thioether) | 2-Benzyl | N—Me—R | S | D | T | L | Pen | threo-b-F—S | bHE | NH2 | |
| 123 | ((thioether) | 2-Benzyl | N—Me—R | S | D | T | L | Pen | threo-b-F—S | bHE | NH2 | |
| 124 | ((thioether) | 2-Benzyl | N—Me—R | S | D | T | L | Pen | B pa | bHE | NH2 | |
| 125 | ((thioether) | 2-Benzyl | N—Me—R | S | D | T | L | Pen | F(3-Me) | bHE | NH2 | |
| 126 | ((thioether) | 2-Benzyl | N—Me—R | S | D | T | L | Pen | F(2-Me) | bHE | NH2 | |
| 127 | ((thioether) | 2-Benzyl | N—Me—R | S | D | T | L | Pen | F(2-CF3)) | bHE | NH2 | |
| 128 | ((thioether) | 2-Benzyl | N—Me—R | S | D | T | L | Pen | b-Me—F | bHE | NH2 | |
| 129 | ((thioether) | 2-Benzyl | N—Me—R | S | D | T | L | Pen | b-Me—F | bHE | NH2 | |
| 130 | ((thioether) | 2-Benzyl | N—Me—R | S | D | T | L | Pen | b-dimethyl-F | bHE | NH2 | |
| 131 | ((thioether) | 2-Benzyl | N—Me—R | S | D | T | L | Pen | b-dimethyl-F | bHE | NH2 | |
| 132 | ((thioether) | 2-Benzyl | N—Me—R | S | D | T | L | Pen | 4-Me—F | bHE | NH2 | |
| 133 | ((thioether) | 2-Benzyl | N—Me—R | S | D | T | L | Pen | Bip | bHE | NH2 | |
| 134 | ((thioether) | 2-Benzyl | N—Me—R | S | D | T | L | Pen | F(4-tBu) | b-H-E | NH2 | |
| 135 | ((thioether) | 2-Benzyl | N—Me—R | S | D | T | L | Pen | F(4tBu) | N—Me-E | NH2 | |
| 136 | ((thioether) | 2-Benzyl | N—Me—R | S | D | T | L | Pen | F(4tBu) | N—Me-D | NH2 | |
| 137 | ((thioether) | 2-Benzyl | N—Me—R | S | D | T | L | Pen | F(4tBu) | alpha-H-E | NH2 | |
| 138 | ((thioether) | 2-Benzyl | Cit | S | D | T | L | Pen | F(4-tBu) | b-H-E | NH2 | |
| 139 | ((thioether) | 2-Benzyl | N—Me—R | A | D | T | L | Pen | F(4-tBu) | b-H-E | NH2 | |
| 140 | ((thioether) | 2-Benzyl | N—Me—R | Abu | D | T | L | Pen | F(4-tBu) | b-H-E | NH2 | |
| 141 | ((thioether) | 2-Benzyl | N—Me—R | Tbu | D | T | L | Pen | F(4-tBu) | b-H-E | NH2 | |
| 142 | ((thioether) | 2-Benzyl | N—Me—R | S | D | T | L | Pen | F(4-tBu) | N—Me-E | OH | |
| 224 | thioether | 2-Benzyl | N—Me—R | S | D | T | L | Pen | W | e | Dap | Ac |
| 225 | thioether | 2-Benzyl | N—Me—R | S | D | T | Nle | Pen | F | e | N—Me-k | NH2 |
| 226 | thioether | 2-Benzyl | N—Me—R | S | D | T | Nle | Pen | W | E | N—Me—K | NH2 |
| 227 | thioether | 2-Benzyl | N—Me—R | S | D | T | Nle | Pen | F | e | N—Me-k | NH2 |
| 228 | thioether | 2-Benzyl | N—Me—R | S | D | T | Nle | Pen | W | E | N—Me-k | NH2 |
| 229 | thioether | 2-Benzyl | N—Me—R | S | D | T | Nle | Pen | F | e | N—Me-k | NH2 |
| 230 | Ac | C(thioether | N—Me—R | S | D | T | L | C(thioether | W | E | k | NH2 |
| | | propane) | | | | | | propane) | | | | |
| 231 | thioether | 2-Benzyl | N—Me—R | S | D | T | L | Pen | W | E | Dab | Ac |
| 232 | thioether | 2-Benzyl | N—Me—R | S | D | T | L | Pen | W | e | Dab | Ac |
| 233 | ((thioether) | 2-Benzyl | N—Me—R | S | D | T | L | Pen | W | E | Dab | NH2 |
| 234 | thioether | 2-Benzyl | N—Me—R | S | D | T | L | Pen | W | E | Dap | Ac |
| 235 | ((thioether) | 2-Benzyl | N—Me—R | S | D | T | L | C | Tic | e | k | NH2 |
| 236 | ((thioether) | 2-Benzyl | N—Me—R | S | D | T | L | Pen | W | f | k | NH2 |
| 237 | ((thioether) | 2-Benzyl | N—Me—R | S | D | T | L | Pen | W | y | k | NH2 |
| 238 | ((thioether) | 2-Benzyl | N—Me—R | S | D | T | L | C | Tic | e | k | NH2 |
| 239 | ((thioether) | 2-Benzyl | N—Me—R | S | D | T | L | Pen | W | P | k | NH2 |
| 240 | ((thioether) | 2-Benzyl | N—Me—R | S | D | T | L | Pen | W | P | K | NH2 |
| 241 | ((thioether) | 2-Benzyl | N—Me—R | S | D | T | L | Pen | W | P | K | NH2 |
| 242 | ((thioether) | 2-Benzyl | N—Me—R | S | D | T | L | Pen | F(2- | e | k | NH2 |
| | | | | | | | | | carbamoyl) | | | |
| 243 | ((thioether) | 2-Benzyl | N—Me—R | S | D | T | L | Pen | F(3- | e | k | NH2 |
| | | | | | | | | | carbamoyl) | | | |
| 244 | ((thioether) | 2-Benzyl | N—Me—R | S | D | T | L | Pen | F(4-COOH) | e | k | NH2 |
| 245 | ((thioether) | 2-Benzyl | N—Me—R | S | D | T | L | Pen | F(2,4-Cl) | e | k | NH2 |
| 246 | ((thioether) | 2-Benzyl | N—Me—R | S | D | T | L | Pen | F(3,4-Cl) | e | k | NH2 |
| 247 | ((thioether) | 2-Benzyl | N—Me—R | S | D | T | L | Pen | F(4-OMe) | e | k | NH2 |
| 248 | ((thioether) | 2-Benzyl | N—Me—R | S | D | T | L | Pen | W | h | k | NH2 |
| 249 | ((thioether) | 2-Benzyl | N—Me—R | S | D | T | L | Pen | W | F(4- | k | NH2 |
| | | | | | | | | | | COOH) | | |
| 250 | ((thioether) | 2-Benzyl | N—Me—R | S | D | T | L | Pen | F(4tBu) | e | k | NH2 |
| 251 | ((thioether) | 2-Benzyl | N—Me—R | S | D | T | L | Pen | F(4-F) | e | k | NH2 |
| 252 | ((thioether) | 2-Benzyl | N—Me—R | S | D | T | L | Pen | Bip | e | k | NH2 |
| 253 | ((thioether) | 2-Benzyl | N—Me—R | S | D | T | L | Pen | W | Tic | k | NH2 |
| 254 | ((thioether) | 2-Benzyl | N—Me—R | S | D | T | L | Pen | W | w | k | NH2 |
| 255 | ((thioether) | 2-Benzyl | N—Me—R | S | D | T | L | Pen | 1-Nal | f | k | NH2 |
| 256 | ((thioether) | 2-Benzyl | N—Me—R | S | D | T | L | Pen | 1-Nal | h | k | NH2 |
| 257 | ((thioether) | 2-Benzyl | N—Me—R | S | D | T | L | Pen | 1-Nal | l | k | NH2 |
| 258 | ((thioether) | 2-Benzyl | N—Me—R | S | D | T | L | Pen | 1-Nal | r | k | NH2 |
| 259 | ((thioether) | 2-Benzyl | N—Me—R | S | D | T | L | Pen | 1-Nal | Tic | k | NH2 |
| 260 | ((thioether) | 2-Benzyl | N—Me—R | S | D | T | L | Pen | 1-Nal | t | k | NH2 |
| 261 | ((thioether) | 2-Benzyl | N—Me—R | S | D | T | L | Pen | 2-Nal | f | k | NH2 |
| 262 | ((thioether) | 2-Benzyl | N—Me—R | S | D | T | L | Pen | 2-Nal | h | k | NH2 |
| 263 | ((thioether) | 2-Benzyl | N—Me—R | S | D | T | L | Pen | 2-Nal | l | k | NH2 |
| 264 | ((thioether) | 2-Benzyl | N—Me—R | S | D | T | L | Pen | 2-Nal | r | k | NH2 |
| 265 | ((thioether) | 2-Benzyl | N—Me—R | S | D | T | L | Pen | 2-Nal | Tic | k | NH2 |
| 266 | ((thioether) | 2-Benzyl | N—Me—R | S | D | T | L | Pen | F(4CF3) | e | k | NH2 |
| 267 | ((thioether) | 2-Benzyl | N—Me—R | S | D | T | L | Pen | Y | e | k | NH2 |
| 268 | ((thioether) | 2-Benzyl | N—Me—R | S | D | T | L | Pen | H | e | k | NH2 |
| 269 | ((thioether) | 2-Benzyl | N—Me—R | S | D | T | L | Pen | F(4tBu) | E | k | NH2 |
| 270 | ((thioether) | 2-Benzyl | N—Me—R | S | D | T | L | Pen | F(4tBu) | b- | k | NH2 |
| | | | | | | | | | | HomoGlu | | |
| 271 | ((thioether) | 2-Benzyl | N—Me—R | S | D | T | L | Pen | F(4-COOH) | E | k | NH2 |
| 272 | ((thioether) | 2-Benzyl | N—Me—R | S | D | T | L | Pen | F(4-COOH) | E | k | NH2 |
| 273 | ((thioether) | 2-Benzyl | N—Me—R | S | D | T | L | Pen | F(4-COOH) | E | k | NH2 |
| 274 | ((thioether) | 2-Benzyl | N—Me—R | S | D | T | L | Pen | F(4-COOH) | b- | k | NH2 |
| | | | | | | | | | | HomoGlu | | |
| 275 | ((thioether) | 2-Benzyl | N—Me—R | S | D | T | L | Pen | F(4-COOH) | b- | k | NH2 |
| | | | | | | | | | | HomoGlu | | |
| 276 | ((thioether) | 2-Benzyl | N—Me—R | S | D | T | L | Pen | F(4-COOH) | b- | k | NH2 |
| | | | | | | | | | | HomoGlu | | |
| 277 | ((thioether) | 2-Benzyl | N—Me—R | S | D | T | L | Pen | Bip | E | k | NH2 |
| 278 | ((thioether) | 2-Benzyl | N—Me—R | S | D | T | L | Pen | Bip | b- | k | NH2 |
| | | | | | | | | | | HomoGlu | | |
| 279 | ((thioether) | 2-Benzyl | N—Me—R | S | D | T | L | Pen | 2-Nal | E | k | NH2 |
| 280 | ((thioether) | 2-Benzyl | N—Me—R | S | D | T | L | Pen | 2-Nal | b- | k | NH2 |
| | | | | | | | | | | HomoGlu | | |
| 281 | ((thioether) | 2-Benzyl | N—Me—R | S | D | T | L | Pen | 1-Nal | E | k | NH2 |
| 282 | ((thioether) | 2-Benzyl | N—Me—R | S | D | T | L | Pen | 1-Nal | b- | k | NH2 |
| | | | | | | | | | | HomoGlu | | |
| 283 | ((thioether) | 2-Benzyl | N—Me—R | S | D | T | L | Pen | 2-Nal | k | NH2 | |
| 284 | ((thioether) | 2-Benzyl | N—Me—R | S | D | T | L | Pen | 2-Nal | k | NH2 | |
| 285 | ((thioether) | 2-Benzyl | N—Me—R | S | D | T | L | Pen | F(4tBu) | E | N—Me—K | NH2 |
| 286 | ((thioether) | 2-Benzyl | N—Me—R | S | D | T | L | Pen | F(4tBu) | E | N—Me-k | NH2 |
| 287 | ((thioether) | 2-Benzyl | N—Me—R | S | D | T | L | Pen | F(4tBu) | b-Homo | N—Me—K | NH2 |
| | | | | | | | | | | Glu | | |
| 288 | ((thioether) | 2-Benzyl | N—Me—R | S | D | T | L | Pen | F(4tBu) | b-Homo | N—Me-k | NH2 |
| | | | | | | | | | | Glu | | |
| 289 | ((thioether) | 2-Benzyl | N—Me—R | S | D | T | L | Pen | Bip | E | N—Me—K | NH2 |
| 290 | ((thioether) | 2-Benzyl | N—Me—R | S | D | T | L | Pen | Bip | E | N—Me-k | NH2 |
| 291 | ((thioether) | 2-Benzyl | N—Me—R | S | D | T | L | Pen | Bip | b-Homo | N—Me—K | NH2 |
| | | | | | | | | | | Glu | | |
| 292 | ((thioether) | 2-Benzyl | N—Me—R | S | D | T | L | Pen | Bip | b-Homo | N—Me-k | NH2 |
| | | | | | | | | | | Glu | | |
| 293 | ((thioether) | 2-Benzyl | N—Me—R | S | D | T | L | Pen | 2-Nal | E | N—Me—K | NH2 |
| 294 | ((thioether) | 2-Benzyl | N—Me—R | S | D | T | L | Pen | 2-Nal | E | N—Me-k | NH2 |
| 295 | ((thioether) | 2-Benzyl | N—Me—R | S | D | T | L | Pen | 2-Nal | b- | N—Me—K | NH2 |
| | | | | | | | | | | HomoGlu | | |
| 296 | ((thioether) | 2-Benzyl | N—Me—R | S | D | T | L | Pen | 2-Nal | b-Homo | N—Me-k | NH2 |
| | | | | | | | | | | Glu | | |
| 297 | ((thioether) | 2-Benzyl | N—Me—R | S | D | T | L | Pen | 1-Nal | E | N—Me—K | NH2 |
| 298 | ((thioether) | 2-Benzyl | N—Me—R | S | D | T | L | Pen | 1-Nal | E | N—Me-k | NH2 |
| 299 | ((thioether) | 2-Benzyl | N—Me—R | S | D | T | L | Pen | 1-Nal | b-Homo | N—Me—K | NH2 |
| | | | | | | | | | | Glu | | |
| 300 | ((thioether) | 2-Benzyl | N—Me—R | S | D | T | L | Pen | 1-Nal | b-Homo | N—Me-k | NH2 |
| | | | | | | | | | | Glu | | |

TABLE 5
Illustrative Thioether Dimers

| SEQ | Peptide | | | | | | | | | | | | | |
|---|---|---|---|---|---|---|---|---|---|---|---|---|---|---|
| ID NO | sequence | 1 | 2 | 3 | 4 | 5 | 6 | 7 | 8 | 9 | 10 | | | Linker |
| 143 | [(thioether) | Acetyl | N—Me—R | S | D | T | L | C | W | k | NH2]2 | | | DIG |
| 144 | [(thioether) | Propionyl | N—Me—R | S | D | T | L | C | W | k | NH2]2 | | | DIG |
| 145 | [(thioether) | 2-Benzyl | N—Me—R | S | D | T | L | C | W | E | k | NH2]2 | | DIG |
| 146 | [(thioether) | 2-Benzyl | N—Me—R | S | D | T | L | Pen | W | E | k | NH2]2 | | DIG |
| 147 | ((thioether) | Acetyl | N—Me—R | S | D | T | L | Pen | W | k | NH2)2 | | | DIG |
| 148 | ((thioether) | Propionyl | N—Me—R | S | D | T | L | Pen | W | k | NH2)2 | | | DIG |
| 149 | [(thioether) | Propionyl | N—Me—R | S | D | T | L | hC | W | k | NH2]2 | | | DIG |
| 150 | ((thioether) | 2-Benzyl | N—Me—R | S | D | T | L | Pen | W | e | k | NH2)2 | | DIG |
| 151 | ((thioether) | 2-Benzyl | N—Me—R | S | D | T | L | Pen | W | b-H-E | k | NH2)2 | | DIG |
| 152 | ((thioether) | 2-Benzyl | N—Me—R | S | D | T | L | Pen | W | E | N—Me-k | NH2)2 | | DIG |
| 153 | ((thioether) | 2-Benzyl | N—Me—R | S | D | T | L | Pen | W | Y | N—Me—K | NH2)2 | | DIG |
| 154 | ((thioether) | 2-Benzyl | N—Me—R | S | D | T | Nle | Pen | W | E | k | NH2)2 | | DIG |
| 155 | ((thioether) | 2-Benzyl | N—Me—R | S | D | T | L | Pen | F | e | k | NH2)2 | | DIG |
| 156 | (thioether) | 2-Benzyl | N—Me—R | S | D | T | L | c | W | b-H-E | k | NH2)2 | | DIG |
| 157 | (thioether) | 2-Benzyl | N—Me—R | S | D | T | L | Hcys | W | E | k | NH2)2 | | DIG |
| 158 | (thioether) | 2-Benzyl | N—Me—R | S | D | T | L | Pen | 1-Nal | e | k | NH2)2 | | DIG |
| 159 | (thioether) | 2-Benzyl | N—Me—R | S | D | T | L | Pen | 2-Nal | e | k | NH2)2 | | DIG |
| 160 | (thioether) | 2-Benzyl | N—Me—R | S | D | T | L | Pen | 1-Nal | e | N—Me—K | NH2)2 | | DIG |
| 161 | (thioether) | 2-Benzyl | N—Me—R | S | D | T | L | Pen | 2-Nal | b-H-E | k | NH2)2 | | DIG |
| 162 | (thioether) | 2-Benzyl | N—Me—R | S | D | T | L | Pen | f | 2-Nal | k | NH2)2 | | DIG |
| 163 | (thioether) | 2-Benzyl | N—Me—R | S | D | T | L | Pen | f | E | k | NH2)2 | | DIG |
| 164 | (thioether) | 2-Benzyl | N—Me—R | S | D | T | L | Pen | F | b-H-E | k | NH2)2 | | DIG |
| 165 | (thioether) | 2-Benzyl | N—Me—R | S | D | T | L | Pen | Y | b-H-E | k | NH2)2 | | DIG |
| 166 | ((thioether) | 2-Benzyl | N—Me—R | S | D | T | L | Pen | F(CF3) | E | k | NH2)2 | | DIG |
| 167 | ((thioether) | 2-Benzyl | N—Me—R | S | D | T | L | Pen | 1Nal | E | k | NH2)2 | | DIG |
| 168 | ((thioether) | 2-Benzyl | N—Me—R | S | D | T | L | Pen | Y | E | k | NH2)2 | | DIG |
| 169 | ((thioether) | 2-Benzyl | N—Me—R | S | D | T | L | Pen | Y | e | k | NH2)2 | | DIG |
| 170 | ((thioether) | 2-Benzyl | N—Me—R | S | D | T | L | Pen | W | e | k | NH2)2 | | ADA |
| 171 | ((thioether) | 2-Benzyl | N—Me—R | S | D | T | L | Pen | W | e | k | NH2)2 | | IDA |
| 172 | (thioether) | 2-Benzyl | N—Me—R | S | D | T | L | Pen(═O) | 2-Nal | e | k | NH2)2 | | DIG |
| 173 | (thioether) | 2-Benzyl | N—Me—R | S | D | T | L | Pen(═O) | 2-Nal | e | k | NH2)2 | | DIG |
| 174 | ((thioether) | 2-Benzyl | N—Me—R | S | D | T | L | Pen | W | e | k | NH2)2 | | IDA- |
| | | | | | | | | | | | | | | Biotine |
| 175 | ((thioether) | 2-Benzyl | N—Me—R | S | D | T | L | Pen | W | e | k | NH2)2 | | IDA- |
| | | | | | | | | | | | | | | PEG4- |
| | | | | | | | | | | | | | | Biotin |
| 176 | ((thioether) | 2-Benzyl | N—Me—R | S | D | T | L | Pen | F(2,4-diCl) | e | k | NH2)2 | | DIG |
| 177 | ((thioether) | 2-Benzyl | N—Me—R | S | D | T | L | Pen | F(3,4-diCl) | e | k | NH2)2 | | DIG |
| 178 | ((thioether) | 2-Benzyl | N—Me—R | S | D | T | L | Pen | Bip | e | k | NH2)2 | | DIG |
| 179 | ((thioether) | 2-Benzyl | N—Me—R | S | D | T | L | c | Aic | e | k | NH2)2 | | DIG |
| 180 | ((thioether) | 2-Benzyl | N—Me—R | S | D | T | L | C | Aic | e | k | NH2)2 | | DIG |
| 181 | ((thioether) | 2-Benzyl | N—Me—R | S | D | T | L | D-Pen | W | E | k | NH2)2 | | DIG |
| 182 | ((thioether) | 2-Benzyl | N—Me—R | S | D | T | L | C | N—Me—Y | E | k | NH2)2 | | DIG |
| 183 | ((thioether) | 2-Benzyl | N—Me—R | S | D | T | L | C | N—Me—F | E | k | NH2)2 | | DIG |
| 184 | ((thioether) | 2-Benzyl | N—Me—R | S | D | T | L | C | Tic | e | k | NH2)2 | | DIG |
| 185 | ((thioether) | 2-Benzyl | N—Me—R | S | D | T | L | c | Tic | e | k | NH2)2 | | DIG |
| 186 | ((thioether) | 2-Benzyl | N—Me—R | S | D | T | L | C | f | E | k | NH2)2 | | DIG |
| 187 | ((thioether) | 2-Benzyl | N—Me—R | S | D | T | L | C | f | e | k | NH2)2 | | DIG |
| 188 | ((thioether) | 2-Benzyl | N—Me—R | S | D | T | L | D-Pen | Y | e | k | NH2)2 | | DIG |
| 189 | ((thioether) | 2-Benzyl | N—Me—R | S | E | T | L | Pen | F | e | k | NH2)2 | | DIG |
| 190 | ((thioether) | 2-Benzyl | N—Me—R | S | D | T | L | Pen | W | e | L | k | NH2)2 | DIG |
| 191 | ((thioether) | 2-Benzyl | N—Me—R | S | D | T | L | Pen | W | e | S | k | NH2)2 | DIG |
| 192 | ((thioether) | 2-Benzyl | N—Me—R | S | D | T | L | Pen | W | e | F | k | NH2)2 | DIG |
| 193 | ((thioether) | 2-Benzyl | N—Me—R | S | D | T | L | Pen | W | e | H | k | NH2)2 | DIG |
| 194 | ((thioether) | 2-Benzyl | N—Me—R | S | D | T | L | Pen | W | e | E | k | NH2)2 | DIG |
| 195 | ((thioether) | 2-Benzyl | N—Me—R | S | D | T | L | Pen | W | e | Y | k | NH2)2 | DIG |
| 196 | ((thioether) | 2-Benzyl | N—Me—R | S | D | T | L | Pen | W | e | I (D-L) | k | NH2)2 | DIG |
| 197 | ((thioether) | 2-Benzyl | N—Me—R | S | D | T | L | Pen | W | e | s | k | NH2)2 | DIG |
| 198 | ((thioether) | 2-Benzyl | N—Me—R | S | D | T | L | Pen | W | e | f | k | NH2)2 | DIG |
| 199 | ((thioether) | 2-Benzyl | N—Me—R | S | D | T | L | Pen | W | e | h | k | NH2)2 | DIG |
| 200 | ((thioether) | 2-Benzyl | N—Me—R | S | D | T | L | Pen | W | e | e | k | NH2)2 | DIG |
| 201 | ((thioether) | 2-Benzyl | N—Me—R | S | D | T | L | Pen | W | e | y | k | NH2)2 | DIG |
| 202 | ((thioether) | 2-Benzyl | N—Me—R | S | D | T | L | Pen | W | Bip | k | NH2)2 | | DIG |
| 203 | ((thioether) | 2-Benzyl | N—Me—R | S | D | T | L | Pen | F | Bip | k | NH2)2 | | DIG |
| 204 | ((thioether) | 2-Benzyl | N—Me—R | S | D | T | L | Pen | F | e | k | OH)2 | | DIG |
| 205 | ((thioether) | 2-Benzyl | N—Me—R | S | D | T | L | C | Tic | Bip | k | NH2)2 | | DIG |
| 206 | (thioether) | 2-Benzyl | N—Me—R | S | D | T | L | Pen | 2-Nal | Bip | k | NH2)2 | | DIG |
| 207 | ((thioether) | 2-Benzyl | N—Me—R | S | D | T | L | C | Tic | e | k | OH)2 | | DIG |
| 208 | ((thioether) | 2-Benzyl | N—Me—R | S | D | T | L | Pen | Bip | e | k | OH)2 | | DIG |
| 209 | ((thioether) | 2-Benzyl | N—Me—R | S | D | T | L | Pen | W | e | k | OH)2 | | DIG |
| 210 | ((thioether) | 2-Benzyl | N—Me—R | S | D | T | L | Pen | W | E(OMe) | k | NH2 | | DIG |
| 211 | ((thioether) | 2-Benzyl | N—Me—R | S | D | T | L | C | Tic | E(OMe) | k | NH2 | | DIG |
| 212 | ((thioether) | 2-Benzyl | N—Me—R | S | D | T | L | Pen | W | e | k | NH2)2 | | IDA- |
| | | | | | | | | | | | | | | Palm |
| 213 | ((thioether) | 2-Benzyl | N—Me—R | S | D | T | L | Pen | W | e | k | NH2)2 | | IDA- |
| | | | | | | | | | | | | | | Lauryl |
| 214 | ((thioether) | 2-Benzyl | N—Me—R | S | D | T | L | Pen | W | e | k | NH2)2 | | IDA- |
| | | | | | | | | | | | | | | oleoyl |
| 215 | ((thioether) | 2-Benzyl | N—Me—R | S | D | T | L | Pen | W | e | k | NH2)2 | | IDA- |
| | | | | | | | | | | | | | | PEG12- |
| | | | | | | | | | | | | | | NH2 |
| 216 | ((thioether) | 2-Benzyl | N—Me—R | S | D | T | L | C | Tic | k | NH2)2 | | | DIG |
| 217 | ((thioether) | 2-Benzyl | N—Me—R | S | D | T | L | Pen | W | e | k | NH2)2 | | IDA- |
| | | | | | | | | | | | | | | PEG12- |
| | | | | | | | | | | | | | | NH- |
| | | | | | | | | | | | | | | oleoyl |
| 218 | ((thioether) | 2-Benzyl | N—Me—R | S | D | T | L | Pen | W | e | k | NH2)2 | | IDA- |
| | | | | | | | | | | | | | | PEG12- |
| | | | | | | | | | | | | | | NH- |
| | | | | | | | | | | | | | | Lauryl |
| 219 | ((thioether) | 2-Benzyl | N—Me—R | S | D | T | L | C | Tic | E | k | NH2)2 | | DIG |
| 220 | ((thioether) | 2-Benzyl | N—Me—R | S | D | T | L | C | Tic | E(OMe) | k | OH)2 | | DIG |
| 221 | ((thioether) | 2-Benzyl | N—Me—R | S | D | T | L | C | Tic | k | OH)2 | | | DIG |
| 222 | ((thioether) | 2-Benzyl | N—Me—R | S | D | T | L | Pen | F(4-tBu) | bHE | k | NH2)2 | | DIG |
| 223 | ((thioether) | 2-Benzyl | N—Me—R | S | D | T | L | Pen | F(4-tBu) | bHE | k | OH)2 | | DIG |
| 301 | (thioether | Butyryl | N—Me—R | S | D | T | L | C | W | k | NH2)2 | DIG | | |
| 302 | (thioether | 2-Benzyl | N—Me—R | S | D | T | L | c | W | b-H-E | k | NH2)2 | DIG | |
| 303 | (thioether | 2-Benzyl | N—Me—R | S | D | T | L | Hcys | W | E | k | NH2)2 | DIG | |
| 304 | thioether | 2-Benzyl | N—Me—R | S | D | T | L | Pen | W | E | Dap | Ac | | |
| 305 | thioether | 2-Benzyl | N—Me—R | S | D | T | L | Pen | W | E | Dab | Ac | | |
| 306 | thioether | 2-Benzyl | N—Me—R | S | D | T | L | Pen | W | e | Dap | Ac | | |
| 307 | thioether | 2-Benzyl | N—Me—R | S | D | T | L | Pen | W | e | Dab | Ac | | |
| 308 | (thioether | 2-Benzyl | N—Me—R | S | D | T | L | Pen | W | e | k | NH2)2 | DIG | |
| 309 | thioether | 2-Benzyl | N—Me—R | S | D | T | L | Pen | W | e | NH2 | | | |
| 310 | (thioether | 3-Benzyl | N—Me—R | S | D | T | L | Pen | W | e | k | NH2)2 | DIG | |
| 311 | (thioether | 4-Benzyl | N—Me—R | S | D | T | L | Pen | W | e | k | NH2)2 | DIG | |
| 312 | ((thioether) | 2-Benzyl | N—Me—R | S | D | T | L | Pen | W | e | k | NH2)2 | IDA- | |
| | | | | | | | | | | | | | PEG12- | |
| | | | | | | | | | | | | | NH- | |
| | | | | | | | | | | | | | Lauryl | |
| 313 | (thioether) | 2-Benzyl | N—Me—R | S | D | T | L | C | Tic | k | OH | | | |
| 314 | ((thioether) | 2-Benzyl | N—Me—R | S | D | T | L | Pen | W | E(OMe) | k | NH2)2 | DIG | |
| 315 | ((thioether) | 2-Benzyl | N—Me—R | S | D | T | L | Pen | 1-Nal | f | k | NH2)2 | DIG | |
| 316 | ((thioether) | 2-Benzyl | N—Me—R | S | D | T | L | Pen | 1-Nal | h | k | NH2)2 | DIG | |
| 317 | ((thioether) | 2-Benzyl | N—Me—R | S | D | T | L | Pen | 1-Nal | l | k | NH2)2 | DIG | |
| 318 | ((thioether) | 2-Benzyl | N—Me—R | S | D | T | L | Pen | 1-Nal | r | k | NH2)2 | DIG | |
| 319 | ((thioether) | 2-Benzyl | N—Me—R | S | D | T | L | Pen | 1-Nal | Tic | k | NH2)2 | DIG | |
| 320 | ((thioether) | 2-Benzyl | N—Me—R | S | D | T | L | Pen | 1-Nal | t | k | NH2)2 | DIG | |
| 321 | ((thioether) | 2-Benzyl | N—Me—R | S | D | T | L | Pen | 1-Nal | E | k | NH2)2 | DIG | |
| 322 | ((thioether) | 2-Benzyl | N—Me—R | S | D | T | L | Pen | 1-Nal | b-HomoGlu | k | NH2)2 | DIG | |
| 323 | ((thioether) | 2-Benzyl | N—Me—R | S | D | T | L | Pen | 1-Nal | E | N—Me—K | NH2)2 | DIG | |
| 324 | ((thioether) | 2-Benzyl | N—Me—R | S | D | T | L | Pen | 1-Nal | E | N—Me-k | NH2)2 | DIG | |
| 325 | ((thioether) | 2-Benzyl | N—Me—R | S | D | T | L | Pen | 1-Nal | b-HomoGlu | N—Me—K | NH2)2 | DIG | |
| 326 | ((thioether) | 2-Benzyl | N—Me—R | S | D | T | L | Pen | 1-Nal | b-HomoGlu | N—Me-k | NH2)2 | DIG | |
| 327 | ((thioether) | 2-Benzyl | N—Me—R | S | D | T | L | Pen | 2-Nal | f | k | NH2)2 | DIG | |
| 328 | ((thioether) | 2-Benzyl | N—Me—R | S | D | T | L | Pen | 2-Nal | h | k | NH2)2 | DIG | |
| 329 | ((thioether) | 2-Benzyl | N—Me—R | S | D | T | L | Pen | 2-Nal | l | k | NH2)2 | DIG | |
| 330 | ((thioether) | 2-Benzyl | N—Me—R | S | D | T | L | Pen | 2-Nal | r | k | NH2)2 | DIG | |
| 331 | ((thioether) | 2-Benzyl | N—Me—R | S | D | T | L | Pen | 2-Nal | Tic | k | NH2)2 | DIG | |
| 332 | ((thioether) | 2-Benzyl | N—Me—R | S | D | T | L | Pen | 2-Nal | E | k | NH2)2 | DIG | |
| 333 | ((thioether) | 2-Benzyl | N—Me—R | S | D | T | L | Pen | 2-Nal | b-HomoGlu | k | NH2)2 | DIG | |
| 334 | ((thioether) | 2-Benzyl | N—Me—R | S | D | T | L | Pen | 2-Nal | k | NH2 | | DIG | |
| 335 | ((thioether) | 2-Benzyl | N—Me—R | S | D | T | L | Pen | 2-Nal | k | NH2 | | DIG | |
| 336 | ((thioether) | 2-Benzyl | N—Me—R | S | D | T | L | Pen | 2-Nal | E | N—Me—K | NH2)2 | DIG | |
| 337 | ((thioether) | 2-Benzyl | N—Me—R | S | D | T | L | Pen | 2-Nal | E | N—Me-k | NH2)2 | DIG | |
| 338 | ((thioether) | 2-Benzyl | N—Me—R | S | D | T | L | Pen | 2-Nal | b-HomoGlu | N—Me—K | NH2)2 | DIG | |
| 339 | ((thioether) | 2-Benzyl | N—Me—R | S | D | T | L | Pen | 2-Nal | b-HomoGlu | N—Me-k | NH2)2 | DIG | |
| 340 | ((thioether) | 2-Benzyl | N—Me—R | S | D | T | L | Pen | Bip | e | k | NH2)2 | DIG | |
| 341 | ((thioether) | 2-Benzyl | N—Me—R | S | D | T | L | Pen | Bip | E | k | NH2)2 | DIG | |
| 342 | ((thioether) | 2-Benzyl | N—Me—R | S | D | T | L | Pen | Bip | b-HomoGlu | k | NH2)2 | DIG | |
| 343 | ((thioether) | 2-Benzyl | N—Me—R | S | D | T | L | Pen | Bip | E | N—Me—K | NH2)2 | DIG | |
| 344 | ((thioether) | 2-Benzyl | N—Me—R | S | D | T | L | Pen | Bip | E | N—Me-k | NH2)2 | DIG | |
| 345 | ((thioether) | 2-Benzyl | N—Me—R | S | D | T | L | Pen | Bip | b-HomoGlu | N—Me—K | NH2)2 | DIG | |
| 346 | ((thioether) | 2-Benzyl | N—Me—R | S | D | T | L | Pen | Bip | b-HomoGlu | N—Me-k | NH2)2 | DIG | |
| 347 | ((thioether) | 2-Benzyl | N—Me—R | S | D | T | L | Pen | F(2,4-Cl) | e | k | NH2)2 | DIG | |
| 348 | ((thioether) | 2-Benzyl | N—Me—R | S | D | T | L | Pen | F(2- | e | k | NH2)2 | DIG | |
| | | | | | | | | | carbamoyl) | | | | | |
| 349 | ((thioether) | 2-Benzyl | N—Me—R | S | D | T | L | Pen | F(3,4-Cl) | e | k | NH2)2 | DIG | |
| 350 | ((thioether) | 2-Benzyl | N—Me—R | S | D | T | L | Pen | F(3- | e | k | NH2)2 | DIG | |
| | | | | | | | | | carbamoyl) | | | | | |
| 351 | ((thioether) | 2-Benzyl | N—Me—R | S | D | T | L | Pen | F(4CF3) | e | k | NH2)2 | DIG | |
| 352 | ((thioether) | 2-Benzyl | N—Me—R | S | D | T | L | Pen | F(4-COOH) | e | k | NH2)2 | DIG | |
| 353 | ((thioether) | 2-Benzyl | N—Me—R | S | D | T | L | Pen | F(4-COOH) | E | k | NH2)2 | DIG | |
| 354 | ((thioether) | 2-Benzyl | N—Me—R | S | D | T | L | Pen | F(4-COOH) | E | k | NH2)2 | DIG | |
| 355 | ((thioether) | 2-Benzyl | N—Me—R | S | D | T | L | Pen | F(4-COOH) | E | k | NH2)2 | DIG | |
| 356 | ((thioether) | 2-Benzyl | N—Me—R | S | D | T | L | Pen | F(4-COOH) | b-HomoGlu | k | NH2)2 | DIG | |
| 357 | ((thioether) | 2-Benzyl | N—Me—R | S | D | T | L | Pen | F(4-COOH) | b-HomoGlu | k | NH2)2 | DIG | |
| 358 | ((thioether) | 2-Benzyl | N—Me—R | S | D | T | L | Pen | F(4-COOH) | b-HomoGlu | k | NH2)2 | DIG | |
| 359 | ((thioether) | 2-Benzyl | N—Me—R | S | D | T | L | Pen | F(4-F) | e | k | NH2)2 | DIG | |
| 360 | ((thioether) | 2-Benzyl | N—Me—R | S | D | T | L | Pen | F(4-OMe) | e | k | NH2)2 | DIG | |
| 361 | ((thioether) | 2-Benzyl | N—Me—R | S | D | T | L | Pen | F(4tBu) | e | k | NH2)2 | DIG | |
| 362 | ((thioether) | 2-Benzyl | N—Me—R | S | D | T | L | Pen | F(4tBu) | E | k | NH2)2 | DIG | |
| 363 | ((thioether) | 2-Benzyl | N—Me—R | S | D | T | L | Pen | F(4tBu) | b-HomoGlu | k | NH2)2 | DIG | |
| 364 | ((thioether) | 2-Benzyl | N—Me—R | S | D | T | L | Pen | F(4tBu) | E | N—Me—K | NH2)2 | DIG | |
| 365 | ((thioether) | 2-Benzyl | N—Me—R | S | D | T | L | Pen | F(4tBu) | E | N—Me-k | NH2)2 | DIG | |
| 366 | ((thioether) | 2-Benzyl | N—Me—R | S | D | T | L | Pen | F(4tBu) | b-HomoGlu | N—Me—K | NH2)2 | DIG | |
| 367 | ((thioether) | 2-Benzyl | N—Me—R | S | D | T | L | Pen | F(4tBu) | b-HomoGlu | N—Me-k | NH2)2 | DIG | |
| 368 | ((thioether) | 2-Benzyl | N—Me—R | S | D | T | L | Pen | H | e | k | NH2)2 | DIG | |
| 369 | ((thioether) | 2-Benzyl | N—Me—R | S | D | T | L | C | Tic | e | k | NH2 | DIG | |
| 370 | ((thioether) | 2-Benzyl | N—Me—R | S | D | T | L | C | Tic | e | k | NH2)2 | DIG | |
| 371 | ((thioether) | 2-Benzyl | N—Me—R | S | D | T | L | Pen | W | E | Dab | NH2)2 | DIG | |
| 372 | ((thioether) | 2-Benzyl | N—Me—R | S | D | T | L | Pen | W | f | k | NH2)2 | DIG | |
| 373 | ((thioether) | 2-Benzyl | N—Me—R | S | D | T | L | Pen | W | y | k | NH2)2 | DIG | |
| 374 | ((thioether) | 2-Benzyl | N—Me—R | S | D | T | L | Pen | W | P | k | NH2)2 | DIG | |
| 375 | ((thioether) | 2-Benzyl | N—Me—R | S | D | T | L | Pen | W | P | K | NH2)2 | DIG | |
| 376 | ((thioether) | 2-Benzyl | N—Me—R | S | D | T | L | Pen | W | p | K | NH2)2 | DIG | |
| 377 | ((thioether) | 2-Benzyl | N—Me—R | S | D | T | L | Pen | W | h | k | NH2)2 | DIG | |
| 378 | ((thioether) | 2-Benzyl | N—Me—R | S | D | T | L | Pen | W | F(4-COOH) | k | NH2)2 | DIG | |
| 379 | ((thioether) | 2-Benzyl | N—Me—R | S | D | T | L | Pen | W | Tic | k | NH2)2 | DIG | |
| 380 | ((thioether) | 2-Benzyl | N—Me—R | S | D | T | L | Pen | W | w | k | NH2)2 | DIG | |
| 381 | thioether | Acetyl | N—Me—R | S | D | T | L | Pen | W | k | NH2)2 | | DIG | |
| 382 | thioether | Propionyl | N—Me—R | S | D | T | L | Pen | W | k | NH2)2 | | DIG | |
| 383 | thioether | Propionyl | N—Me—R | S | D | T | L | hC | W | k | NH2)2 | | DIG | |
| 384 | ((thioether) | 2-Benzyl | N—Me—R | S | D | T | L | Pen | Y | e | k | NH2)2 | DIG | |

### Example 2

### Characterization of Thioether Peptide Monomer and Dimer Molecules

The stability, potency, and selectivity of certain thioether peptide monomer and dimers were determined using a variety of assays described herein. Peptides listed in Table 8 can be used as control peptides for all of the assays described herein.

### Simulated Intestinal Fluid (SIF) Stability Assay

Studies were carried out in simulated intestinal fluid (SIF) to evaluate intestinal stability of the peptide molecules of the instant invention. To prepare the SIF reagent, blank FASSIF was prepared by dissolving 0.348 g NaOH, 3.954 g sodium phosphate monobasic monohydrate and 6.186 g NaCl in a final volume of 1 liter water (final pH=6.5). To this solution, 24 g porcine pancreatin (Sigma catalog P7545) was added and stirred for 30 minutes (final pancreatin concentration is 2.4%). The solution was filtered through a cheese cloth and a No. 1 Whatman filter, and 10 ml aliquots were stored at −70° C. To run the reaction, a 10 ml aliquot was thawed at 37° C., and 125 μl aliquots were removed and mixed with an equal volume of blank FASSIF. The peptide stock solution (10 mM in 100% DMSO) was diluted 75-fold in blank FASSIF. A 50 μl aliquot of the diluted peptide was combined with 125 μl pancreatin (2.4%) and 125 μl blank FASSIF to yield final concentrations of 1% pancreatin and 22 μM peptide. The reactions were incubated at 37° C., and at various time points 50 μl aliquots were removed and added to 200 μl of quench solution containing 50% acetonitrile, 50% methanol, 5% formic acid, and 1 μg/ml internal standard. The quenched samples were centrifuged at 10,000 rpm for 10 minutes, and the supernatants were analyzed by LCMS/MS. The percent remaining at each time point was calculated based on the peak area response ratio of test to compound to internal standard. Half-lives were calculated by fitting to a first-order exponential decay equation using GraphPad. A small sampling of the results of these studies is provided and discussed herein and in the accompanying figures.

### Simulated Gastric Fluid (SGF) Stability Assays

Studies were carried out in simulated gastric fluid (SGF) to evaluate intestinal stability of the peptide molecules of the instant invention. SGF was prepared by adding 20 mg NaCl, 32 mg porcine pepsin (MP Biochemicals, catalog 02102599), and 70 μl HCl to 10 ml water (final pH=2). Aliquots of SGF (0.5 ml each) were pre-warmed at 37° C. To start the reaction, 1 μl of peptide stock solution (10 mM in DMSO) was added to 0.5 ml SGF and thoroughly mixed such that the final peptide concentration was 20 μM. The reactions were incubated at 37° C. with gentle shaking. At each time point (0, 15, 30, 60 min) 50 μl aliquots were removed and added to 200 ul acetonitrile containing 0.1% formic acid to quench the reaction. Samples are stored at 4° C. until the end of the experiment and centrifuged at 10,000 rpm for 5 minutes. Aliquots of the supernatant were removed, diluted 1:1 into distilled water containing internal standard, and analyzed by LCMS/MS. Percent remaining at each timepoint was calculated based on the peak area response ratio of test to compound to internal standard. Time 0 was set to 100%, and all later timepoints were calculated relative to time 0. Half-lives were calculated by fitting to a first-order exponential decay equation using GraphPad.

### Redox Stability Assays

Studies were carried out under redox conditions to evaluate intestinal stability of the peptide molecules of the instant invention.

### Dithiothreitol (DTT) Redox Stability Assay

The DTT stability assay was prepared by adding 5 μl of a 10 mM peptide stock solution in DMSO to 1 ml of 100 mM Tris-Cl, pH 7.5 (final peptide concentration is 50 μM). At time 0 min, 5 ul of a freshly thawed 100 mM DTT solution was added such that the final DTT concentration is 0.5 mM. The reactions were incubated at room temperature. At different time points up to 120 minutes, 50p aliquots were removed and the reaction was quenched by adding 10 μl of 5M acetic acid. To measure disappearance of the parent peptide, the quenched samples (30 μl) were analyzed by reverse phase HPLC and UV absorbance at 220 nm. Half-lives were calculated by fitting to a first-order exponential decay equation using Excel.

### Cysteine/Cystine Redox Stability Assay

Peptides were diluted to 90 μM by adding 4.545 μl of a 10 mM peptide DMSO stock to 495.45 μl of 100 mM Tris-Cl, pH 7.5. Aliquots of 55 μl were removed and added to 20 μl of 2.5 mM Cystine in 100 mM Tris-Cl, pH 7.5. Cysteine stock solutions in 100 mM Tris-Cl, pH 7.5 were prepared fresh at the following concentrations: 400 mM, 200 mM, 80 mM, 44 mM, 22 mM, 11 mM, 5.5 mM and blank. At time 0, 25 μl of each cysteine stock solution was added to the 55 μl of cystine/peptide solution and the mixture was incubated at room temperature for 40 min. The samples were quenched by adding 20 μl of 5M acetic acid and analyzed by reverse phase HPLC. The fraction of oxidized peptide was calculated and plotted against the calculated oxidation reduction potential (OEP) as defined by the Nernst equation.

### α4β7-MAdCAM Competition ELISA

A nickel coated plate (Pierce #15442) was coated with rh integrin α4β7 (R&D Systems #5397-A30) at 800 ng/well and incubated at room temperature with shaking for hr. The solution was then removed by shaking and blocked with assay buffer (50 mM Tris-HCl pH7.6, 150 mM NaCl, 1 mM MnCl₂ or MgCl₂, 0.05% Tween-20 and 0.5% BSA) at 250 ul/well. The plate was then incubated at room temperature for 1 hr. Each well was washed 3 times with wash buffer (50 mM Tris-HCl pH7.6, 100 mM NaCl, 1 mM MnCl₂ or MgCl₂, 0.05% Tween-20). To each well was added 25 ul of a serial dilution (3-fold dilutions in assay buffer) of peptides starting at 20 μM. 25 ul of recombinant human MAdCAM-1 (R&D Systems #6056-MC) was then added to each well at a fixed concentration 20 nM. The final starting peptide concentration was 10 μM, and the final MAdCAM-1 concentration was 10 nM. The plates were then incubated at room temperature for 1 hr to reach binding equilibrium. The wells were then washed three times with wash buffer. 50 ul of mouse anti-human IgG1-HRP (Invitrogen #A10648) diluted in 1:2000 in assay buffer was then added to each well. The wells were incubated at room temperature for 45 min with shaking. The wells were then washed 3 times with wash buffer. 100 ul of TMB were then added to each well and closely observe during development time. The reaction was stopped with 2N H₂SO₄ and absorbance was read at 450 nm.

### α4β1-VCAM Competition ELISA

A Nunc MaxiSorp plate was coated with rh VCAM-1/CD106 Fc chimera (R&D #862-VC) at 400 ng/well in 50 ul per well in 1×PBS and incubated overnight at 4° C. The solution was removed by shaking and then blocked with 250 ul of 1% BSA in 1×PBS per well. The wells were then incubated at room temperature for 1 hr with shaking. Each well was then washed once with wash buffer (50 mM Tris-HCl pH7.6, 100 mM NaCL, 1 mM MnCl₂ or MgCl₂, 0.05% Tween-20). 25 ul of serial dilutions of peptides starting at 200 μM in assay buffer (Assay buffer: 50 mM Tris-HCl pH7.6, 100 mM NaCl, 1 mM MnCl₂ or MgCl₂, 0.05% Tween-20) was added to each well. Additionally, 25 ul of α4β1 (R&D Systems #5668-A4) was added to each well at a fixed concentration of 120 nM. The final peptide and α4β1 concentrations were 100 μM and 60 nM, respectively. The plates were then incubated at 37° C. for 2 hr. The solution was then removed by shaking and each well was washed three times with wash buffer. 50 ul of 9F10 antibody at 4 ug/ml (purified mouse anti-human CD49d, BD Bioscience Cat #555502) was then added to each well, and the plate was incubated at room temperature for 1 hr with shaking. The solution was again removed by shaking, and each well was washed three times with wash buffer. 50 ul of peroxidase-conjugated AffiniPure Goat anti-mouse IgG (Jackson immune research cat #115-035-003) diluted in 1:5000 in assay buffer was added to each well. The plate was incubated at room temperature for 30 min with shaking. Each well was then washed 3 times with wash buffer. 100 ul of TMB was then added to each well and closely observe during developing time. The reaction was stepped with 2N H₂SO₄ and absorbance was read at 450 nm.

### PBMC Memory T Cell Adhesion Assay

Fresh CD4+/CD45RO+ memory T cells were isolated from human peripheral blood mononuclear cell (PBMC) donors by Aragen Bioscience Inc. (Morgan Hill, CA). The assay plate was prepared using IgG Fc capture antibody (donkey anti human) immobilized at 500 ng/well in 50 mM sodium bicarbonate buffer, pH 9.5, ON, 4 C onto a Greiner Fluotrac plate (100 ul per well). The plate was rinsed two time with Blocking Buffer (25 mM Tris HCl, pH7.5, 150 mM NaCl, 1.5% BSA, 0.05% Tween), and blocked with Blocking Buffer for 2 hours at 37 C or 5 hours at RT using 200 ul per well. The Blocking Buffer was removed and either MAdCAM-1 or VCAM-1 at 400 ng/well in Blocking Buffer was added and the plate incubated overnight at 4 C (100 ul per well). The plate was washed two times with Blocking Buffer, and rinsed once with 200 ul Binding Media (DMEM phenol red free, 10 mM HEPES, 1× Na pyruvate, 1× Glutamine, and supplemented with 1 mM MnCl₂ prior to use). To prepare cells, approximately 25 million CD4+/CD45RO+ memory T cells were counted by trypan blue exclusion using a haemocytometer to determine viability and cell count. The cells were transferred to a 50 ml conical tube, and centrifuged at 1200 rpm for 10 minute. The media was aspirated and the cell pellet resuspended in 15 ml Binding Media. The cells were centrifuged again and resuspended in the appropriate amount of Binding Media to be used for assays (50 ul of cells per well at 2× the final density). To each well, and equal volume (50 ul) of test compound was added and the plate was incubated for 1.5 hours at 37 C, 5% CO2. Each well was rinsed 3× with 150 ul per well of Binding Media. CyQuant NF reagent was prepared as suggested by manufacturer), and 100 ul of CyQuant NF reagent was added per well. The plate was incubated at 37 C, 5% CO2, for 45 minutes. The plate was protected from light by using black adhesive seals. Fluorescence intensity was measured using a Molecular Devices Gemini EM Fluorescent Plate Reader (Ex 485/Em530, Bottom Read, Reading Sensitivity=20). IC50 curves are generated using Graph Pad Prism and the curves analyzed using analyzed using a non-linear regression (four parameters) algorithm. The log (concentration) versus RFU (Ex485/Em530) was plotted to determine IC50 values.

### α4β7-MAdCAM Cell Adhesion Assay

RPMI 8866 cells (Sigma #95041316) were cultured in RPMI 1640 HEPES medium (Invitrogen #22400-089) supplemented with 10% serum (Fetal Bovine Serum, Invitrogen #16140-071), 1 mM sodium pyruvate (Invitrogen #11360-070), 2 mM L-glutamine (Invitrogen #25030-081) and Penicillin-Streptomycin (Invitrogen #15140-122) at 100 units of penicillin and 100 pg of streptomycin per ml. The cells were washed two times in DMEM medium (ATCC #30-2002) supplemented with 0.1% BSA, 10 mM HEPES pH 7 and 1 mM MnCl₂. The cells were re-suspended in supplemented DMEM medium at a density of 4×10⁶ cells/ml.

A Nunc MaxiSorp plate was coated with rh MAdCAM-1/Fc Chimera (R&D #6065-MC) at 200 ng per well in 50 ul per well in 1×PBS and incubated at 4° C. overnight. The solution was then removed by shaking, blocked with 250 ul per well PBS containing 1% BSA, and incubated at 37° C. for 1 hr. The solution was removed by shaking. Peptides were diluted by serial dilution in a final volume of 50 ul per well (2× concentration). To each well, 50 ul of cells (200,000 cells) were added and the plate was incubated at 37° C., 5% CO2 for 30-45 min to allow cell adhesion. The wells were washed manually three times (100 ul per wash) with supplemented DMEM. After the final wash, 100 ul/well of supplemented DMEM and 10 ul/well of MTT reagent (ATTC cat #30-1010K) were added. The plate was incubated at 37° C., 5% CO2 for 2-3 hrs until a purple precipitate is visible. 100 ul of Detergent Reagent (ATTC cat #30-1010K) was added to each well. The plate was covered from the light, wrapped in Parafilm to prevent evaporation, and left overnight at room temperature in the dark. The plate was shaken for 5 min and the absorbance at 570 nm is measured. To calculate the dose response, the absorbance value of control wells not containing cells was subtracted from each test well.

### α4β1-VCAM Cell Adhesion Assay

Jurkat E6.1 cells (Sigma #88042803) were cultured in RPMI 1640 HEPES medium (Invitrogen #22400-089) supplemented with 10% serum (Fetal Bovine Serum, Invitrogen #16140-071), 1 mM sodium pyruvate (Invitrogen #11360-070), 2 mM L-glutamine (Invitrogen #25030-081) and Penicillin-Streptomycin (Invitrogen #15140-122) at 100 units of penicillin and 100 pg of streptomycin per ml. The cells were washed two times in DMEM medium (ATCC #30-2002) supplemented with 0.1% BSA, 10 mM HEPES pH 7 and 1 mM MnCl₂. The cells were re-suspended in supplemented DMEM medium at a density of 4×10⁶ cells/ml.

A Nunc MaxiSorp plate was coated with rh VCAM-1/CD106 Fc chimera (R&D #862-VC) at 400 ng per well in 50 ul per well in 1×PBS and incubated at 4° C. overnight. The solution was then removed by shaking, blocked with 250 ul per well PBS containing 1% BSA, and incubated at 37° C. for 1 hr. The solution was removed by shaking. Peptides were diluted by serial dilution in a final volume of 50 ul per well (2× concentration). To each well, 50 ul of cells (200,000 cells) were added and the plate was incubated at 37° C., 5% C02 for 30-45 min to allow cell adhesion. The wells were washed manually three times (100 ul per wash) with supplemented DMEM. After the final wash, 100 ul/well of supplemented DMEM and 10 ul/well of MTT reagent (ATTC cat #30-1010K) were added. The plate was incubated at 37° C., 5% C02 for 2-3 hrs until a purple precipitate is visible. 100 ul of Detergent Reagent (ATTC cat #30-1010K) is added to each well. The plate was covered from the light, wrapped in Parafilm to prevent evaporation, and left overnight at room temperature in the dark. The plate was shaken for 5 min and the absorbance at 570 nm is measured. To calculate the dose response, the absorbance value of control wells not containing cells was subtracted from each test well.

The potency, selectivity and stability data for certain illustrative peptide monomers and dimers of the present invention are provided in Tables 6 and 7. These peptides have the structures shown in Tables 4 and 5, which may be identified by their SEQ ID NOs. Table 6 provides potency, selectivity and stability data for representative peptide monomers. Table 7 provides potency, selectivity and stability data for representative peptide dimers. For potency, IC50 values are shown as * <25 nM **=25-100 nM, ***=100-1000 nM. Where data not shown, data was not determined, but is is expected that these peptides have an IC50<100 nM in α4β7 ELISA and/or cell assays.

TABLE 6
Characterization of Illustrative Thioether Monomer Peptides

| SEQ | ELISA | ELISA | Cell-Adhesion | PBMC | SIF (Porcine) | SGF (Porcine) |
|---|---|---|---|---|---|---|
| ID NO | A4B7(nM) | A4B1(nM) | A4B7(nM) | IC50(nM) | (half-life, min) | (Half -life, Min) |
| 49 | ** | | >1000 | | | |
| 50 | *** | | | | | |
| 51 | ** | | | | 6 | |
| 52 | >1000 | | | | | |
| 53 | *** | | | | >180 | |
| 54 | >1000 | | | | second | |
| 55 | * | *** | *** | | 25 | |
| 56 | * | *** | *** | | 186 | |
| 57 | *** | | | | <20 | |
| 58 | >1000 | | | | | |
| 59 | *** | | | | <20 | |
| 60 | * | *** | | | >180 | |
| 61 | * | | | | >180 | |
| 62 | * | | | | >180 | |
| 63 | * | | | | >180 | |
| 64 | * | | | | 179 | |
| 65 | * | | ** | | >180 | |
| 66 | ** | | | | >180 | |
| 67 | * | | | | <20 | |
| 68 | * | | | | >180 | |
| 69 | * | | *** | | >180 | |
| 70 | * | | | | >180 | |
| 71 | ** | | | | >180 | |
| 72 | * | | | | >180 | |
| 73 | * | | | | >180 | |
| 74 | * | | | | >180 | |
| 75 | * | | | | >180 | |
| 76 | * | | | | >180 | |
| 77 | * | | | | 88 | |
| 78 | * | | | | 78 | |
| 79 | * | | | | | |
| 80 | * | | ** | | | |
| 81 | * | *** | ** | | | |
| 82 | * | | *** | | | |
| 83 | * | | | | | |
| 84 | * | | | | | |
| 85 | * | | | | | |
| 86 | * | | | | | |
| 87 | * | | | | | |
| 88 | * | | ** | | | |
| 89 | * | | *** | | | |
| 90 | * | | | | | |
| 91 | * | | *** | | | |
| 92 | * | | ** | | | |
| 93 | * | | ** | | | |
| 94 | * | | *** | | >180 | |
| 95 | * | | *** | | >180 | |
| 96 | * | | *** | | 26 | |
| 97 | * | *** | ** | | >180, >180 | >180 |
| 98 | * | *** | ** | *** | >300 | >180 |
| 99 | * | | | | | |
| 100 | * | | ** | | | |
| 101 | * | | *** | *** | | |
| 102 | * | | ** | | | |
| 103 | * | | | | | |
| 104 | * | | ** | | | |
| 105 | * | | | | | |
| 106 | * | | ** | | | |
| 107 | * | | ** | | | |
| 108 | * | | ** | | | |
| 109 | * | | | | | |
| 110 | * | | ** | | | |
| 111 | | | >1000 | | | |
| 112 | | | >1000 | | | |
| 113 | * | | ** | | | |
| 114 | * | ** | ** | | >180 | |
| 115 | * | | | | | |
| 116 | * | | ** | | | |
| 117 | | | *** | | | |
| 118 | | | *** | | | |
| 119 | | | *** | | | |
| 120 | | | *** | | | |
| 121 | | | *** | | | |
| 122 | | | *** | | | |
| 123 | | | ** | | | |
| 124 | | | ** | | | |
| 125 | | | * | *** | >180 | |
| 126 | | | ** | | >180 | |
| 127 | | | ** | | | |
| 128 | | | ** | | | |
| 129 | | | ** | | | |
| 130 | | | *** | | | |
| 131 | | | ** | | | |
| 132 | | | * | *** | >180 | |
| 133 | | | * | *** | >180 | |
| 134 | | | * | ** | >180(428) | |
| 135 | | | * | *** | | |
| 136 | | | ** | | | |
| 137 | | | ** | | | |
| 138 | | | *** | | | |
| 139 | | | ** | | | |
| 140 | | | >1000 | | | |
| 141 | | | >1000 | | | |
| 142 | | | * | | | |

TABLE 7
Characterization of Illustrative Thioether Peptide Dimers

| SEQ | ELISA | ELISA | Cell-Adhesion | Cell Adhesion | PBMC | SIF (Porcine) | SGF (Porcine) |
|---|---|---|---|---|---|---|---|
| ID NO | A4B7(nM) | A4B1 (nM) | A4B7 (nM) | A4B1 (nM) | IC50 (nM) | (half-life, min) | (Half-life, Min) |
| 143 | * | >1000 | *** | | | | |
| 144 | * | >1000 | >1000 | | | <20 | |
| 145 | | *** | ** | | | | |
| 146 | * | ** | * | | | <20 | |
| 147 | | | >1000 | | | | |
| 148 | | | >1000 | | | | |
| 149 | | | >1000 | | | <20 | |
| 150 | * | ** | * | >100,000 | | >180, >180, | >180 |
| | | | | | | >300 | |
| 151 | * | ** | * | | | >180 | |
| 152 | * | ** | * | >100,000 | >180 | | >60 |
| 153 | | *** | * | | | >180 (275) | |
| 154 | * | ** | * | >100,000 | | <20 | |
| 155 | * | *** | * | | ** | >180, >300 | >180 |
| 156 | | ** | ** | >100,000 | | >180 | |
| 157 | | *** | ** | | | <20 | |
| 158 | * | *** | * | >100,000 | | >180 | |
| 159 | * | ** | * | >100,000 | | >180 | >180 |
| 160 | * | | * | | | >180 | >60 |
| 161 | * | ** | * | >100,000 | | >180 | |
| 162 | | | *** | | | >180 | |
| 163 | | | * | | | >180 | |
| 164 | * | ** | * | >100,000 | | >180 | >60 |
| 165 | * | ** | * | >100,000 | | >180 | >60 |
| 166 | * | ** | * | | | 30 | |
| 167 | * | ** | * | >100,000 | | <20 | |
| 168 | * | ** | * | >100,000 | | | |
| 169 | * | *** | * | >100,000 | | >180, >180 | >180 |
| 170 | | | * | | | | |
| 171 | | | * | | | | |
| 172 | | | >1000 | | | | |
| 173 | | | ** | | | | |
| 174 | | | * | | | | |
| 175 | | | * | | | | |
| 176 | | | * | | | | |
| 177 | | | * | | | | |
| 178 | * | *** | * | >1000 | | >180 (375), | >180 |
| | | | | | | >180 (266), >180 | |
| 179 | | | *** | | | | |
| 180 | | | *** | | | | |
| 181 | | | >1000 | | | | |
| 182 | | | ** | | | | |
| 183 | | | ** | | | | |
| 184 | * | *** | * | >100,000 | ** | >180, >180, | >180 |
| | | | | | | >180 | |
| 185 | | | ** | | | | |
| 186 | | | ** | | | | |
| 187 | | | ** | | | | |
| 188 | | | >1000 | | | | |
| 189 | | | >1000 | | | | |
| 190 | | | * | | | | |
| 191 | | | * | >100,000 | | | |
| 192 | | | * | | | | |
| 193 | | | * | | | | |
| 194 | | *** | * | | | | |
| 195 | | | * | | | | |
| 196 | | | * | | | | |
| 197 | | | * | | | | |
| 198 | | | * | | | | |
| 199 | | | * | | | | |
| 200 | | | * | | | | |
| 201 | | | * | | | | |
| 202 | | | *** | | | | |
| 203 | | | *** | | | | |
| 204 | * | *** | * | >100,000 | | >180 | >180 |
| 205 | | | *** | | | | |
| 206 | | | >1000 | | | | |
| 207 | * | *** | * | >100,000 | | >180 | >180 |
| 208 | * | ** | * | >100,000 | | >180 (312) | >180 |
| 209 | * | *** | * | >100,000 | | >180 | |
| 210 | | | *** | | | | |
| 211 | | | * | | | 7 | |
| 212 | | | ** | | | | |
| 213 | | | * | | | >180 (419) | |
| 214 | | | ** | | | | |
| 215 | | | * | | | | |
| 216 | | | * | | | >180 | |
| 217 | | | ** | | | | |
| 218 | | | * | | | | |
| 219 | | | * | | | >180, 407 | >360 |
| 220 | | | * | | | >180 | |
| 221 | | | ** | | | | |
| 222 | | | * | | * | | |
| 223 | | | * | | * | | |

TABLE 8
Characterization of Illustrative Peptide Monomers

| | | | | | | | | | | | | | | | | Redox |
|---|---|---|---|---|---|---|---|---|---|---|---|---|---|---|---|---|
| SEQ | Peptide | | | | | | | | | | | ELISA | ELISA | Cell-Adhesion | SIF (Porcine) | stability |
| ID NO | sequence | 1 | 2 | 3 | 4 | 5 | 6 | 7 | 8 | 9 | 10 | A4B7 (nM) | A4B1 (nM) | A4B7 (nM) | (half-life, min) | (DTT) |
| 385 | Ac | C | R | S | D | T | L | C | G | E | NH2 | 97 | 2020 | 590 | <1 min | ~3 min |
| 386 | Ac | C | R | S | D | T | L | C | NH2 | | | 96.8 | 2880 | 1221 | <1 min | ~3 min |

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A method for treating or preventing inflammatory bowel disease (IBD), the method comprising administering to a subject in need thereof an effective amount of a peptide molecule comprising a structure of Formula (V):
(Formula (V))
(SEQ ID NO: 49)
Xaa¹-Xaa²-Xaa³-Xaa⁴-Xaa⁵-Xaa⁶-Xaa⁷-Xaa⁸-Xaa⁹-
Xaa¹⁰-Xaa¹¹-Xaa¹²-Xaa¹³-Xaa¹⁴
or a pharmaceutically acceptable salt thereof, wherein the peptide comprises a thioether bond between Xaa⁴ and Xaa¹⁰, and wherein:
Xaa¹ is absent, or Xaa¹ is any amino acid;
Xaa² is absent, or Xaa² is any amino acid;
Xaa³ is absent, or Xaa³ is any amino acid;
Xaa⁴ is an amino acid, aliphatic acid, alicyclic acid, or modified 2-methyl aromatic acid having a side chain with one or two carbons, and capable of forming a thioether bond with Xaa¹⁰;
Xaa⁵ is selected from the group consisting of N(alpha)-Me-Arg, Arg, HomoArg, Dap, Dab, Arg-Me-sym, Arg-Me-asym, 4-Guan, Cit, Cav, N-Me-Lys, Phe(4-quanidino), Phe(4-carbamoyl amino), Phe(4-NH₂), N-Me-HomoArg, Tyr, His, and suitable isostere replacements;
Xaa⁶ is selected from the group consisting of Ser, Gly, Thr, Ile, and suitable isostere replacements;
Xaa⁷ is selected from the group consisting of Asp, N-Me-Asp, Asp(OMe), D-Asp, and suitable isostere replacements;
Xaa⁸ is selected from the group consisting of Thr, Gln, Ser, Asp, Pro, Gly, His, Ala, Ile, Phe, Lys, Arg, Asn, Glu, Val, Tyr, Trp, Leu, Met, HomoLeu, Nle, and N-Methyl amino acids including N-Me-Thr;
Xaa⁹ is selected from the group consisting of Gln, Asn, Asp, Pro, Gly, Ala, Phe, Leu, Glu, Ile, Val, HLeu, n-Butyl Ala, n-Pentyl Ala, n-Hexyl Ala, Nle, cyclobutyl-Ala, Cpa, Aoc, N-Me-Leu, and suitable isostere replacements;
Xaa¹⁰ is selected from the group consisting of Cys, N-Me-Cys, D-Cys, HCys, Pen, D-Pen, and Pen(═O);
Xaa¹¹ is absent or is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Phe (2-carbomyl), Tyr(Me), HomoPhe, N-Me-Phe, N-Me-Tyr, Ser, Sar, Dihydro Trp, Ile, Leu, Ser, Arg, Thr, Sar, and Ser, aromatic amino acids, substituted aromatic amino acids, Gly, Gln, Asn, Asp, Ala, Ile, Leu, Val, Met, Thr, Lys, Trp, Tyr, His, Glu, Ser, Arg, Pro, Phe, Sar, 1-Nal, 2-Nal, D-1-Nal, D-2-Nal, HPhe, D-Phe, D-Tyr, Phe(4-F), O-Me-Tyr, dihydro-Trp, Dap, Dab, Dab(Ac), Orn, D-Orn, N-Me-Orn, N-Me-Dap, D-Dap, D-Dab, Bip, Ala(3,3diphenyl), Biphenyl-Ala, aromatic ring substituted Phe, aromatic ring substituted Trp, aromatic ring substituted His, hetero aromatic amino acids, N-Me-Lys, N-Me-Lys(Ac), 4-Me-Phe, Phe(4tBu), Phe(4-OMe), Phe(4-COOH), Phe(2-carbomyl), Phe(3-carbomyl), Phe(CF3), Phe(2,4-diCl), Phe(3,4-diCl), Aic, N-Me-Tyr, N-Me-Phe, Tic, Phe(4CF3), Bpa, Phe(3-Me), Phe(2-Me), Phe(2-CF3), β-Me-Phe, and corresponding D-amino acids and suitable isostere replacements;
Xaa¹² is absent or selected from the group consisting of aromatic amino acids, substituted aromatic amino acids, Glu, D-Glu, HomoGlu, Beta-Homo-Glu, Asp, D-HomoGlu, Amide, Lys, COOH, CONH₂, Gln, Pro, Gly, His, Ala, Ile, Phe, Arg, Leu, Val, Tyr, Trp, Met, Gla, Ser, Asn, D-Glu, β-HGlu, 2-Nal, 1-Nal, D-Asp, Bip, β-HPhe, β-Glu, D-Tyr, D-Phe, D-Lys, Dap, Dab, Orn, D-Orn, N-Me-Orn, N-Me-Dap, N-Me-Dab, N-Me Lys, D-Dap, D-Dab, D-His, F(4-COOH), Tic, D-Trp, D-Leu, D-Arg, D-Thr, N-Me-Glu, N-Me-Asp, alpha-H-Glu, isosteres, and corresponding D-amino acids;
Xaa¹³ is absent or any amino acid; and
Xaa¹⁴ is absent or any amino acid;
wherein if the peptide molecule is a peptide dimer or subunit thereof, then Xaa¹⁴ is absent or selected from the group consisting of: any amino acid with an amine side chain, Lys, D-Lys, N-Me-Lys, D-N-Me-Lys, Orn, N-Me-Orn, Dab, N-Me-Dab, Dap, N-Me-Dap, Homo-Lys, D-Dap, D-Dab, D-Orn, Gln, Pro, Gly, His, Ala, Ile, Phe, Lys, Arg, Leu, Val, Tyr, Trp, Met, Glu, Ser, Asn, Gla, Cys, HomoCys, COOH, CONH₂, suitable isosteres, corresponding D-amino acids, and corresponding N-Methyl amino acids, and
wherein the peptide molecule comprises a thioether bond between Xaa⁴ and Xaa¹⁰.

2. The method of claim 1, wherein
Xaa¹ is absent or any amino acid;
Xaa² is absent or any amino acid;
Xaa³ is absent or any amino acid;
Xaa⁴ is a 2-methyl-benzoyl moiety or a modified HomoSer, optionally Homo-Ser-Cl;
Xaa⁵ is selected from the group consisting of: N-Me-Arg, Arg, N-Me-Lys, Phe (4-quanidino), Phe(4-carbonylamino), Cit, Phe(4-NH₂), N-Me-Homo-Arg, Homo-Arg, Tyr and His;
Xaa⁶ is Ser, Gly, Thr or Ile;
Xaa⁷ is Asp or D-Asp;
Xaa⁸ is selected from the group consisting of: Thr, Val, Ile, Leu, hLeu, Nle, and Val;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys; and
Xaa¹¹ is absent or selected from the group consisting of: Trp, Phe, 2-Nal, 1-Nal, Tyr, His, Phe(4-F), Phe(4-CF3), Phe (4-CH3), Phe (4-tfu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Phe(4-carbomyl), Phe(3-Carbomyl), Phe (2-carbomyl), Tyr(Me), HomoPhe, N-Me-Phe, N-Me-Tyr, Ser, Sar, Dihydro Trp, Ile, Leu, Arg, Thr, Sar, Bpa, Phe(3-Me), Phe(2-Me), Phe(2-CF3), β-Me-Phe, and Ser;
Xaa¹² is absent or selected from the group consisting of: Glu, D-Glu, homoGlu, Asp, D-Asp, D-homoGlu, D-Asp, Gla, beta-homo-Glu, N-Me-Glu, N-Me-Asp, alpha-H-Glu, corresponding D-amino acid, and isosteres;
Xaa¹³ is absent or any amino acid; and
Xaa¹⁴ is any amino acid.

3. The method of claim 1, wherein
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser, Gly, Thr, or Ile;
Xaa⁷ is Asp or D-Asp;
Xaa⁸ is selected from the group consisting of: Thr, Val, Ile, Leu, hLeu and Nle;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Pge(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Bpa, Phe(3-Me), Phe(2-Me), Phe(2-CF3), β-Me-Phe, Ser and any substituted aromatic amino acid and corresponding D-amino acids;
Xaa¹² is selected from the group consisting of: any aromatic amino acid, Glu, D-Glu, homoGlu, Asp, D-Asp, D-homoGlu, Gla, beta-homo-Glu, N-Me-Glu, N-Me-Asp, alpha-H-Glu corresponding D-amino acid and isosteres;
Xaa¹³ is absent; and
Xaa¹⁴ is any amino acid.

4. The method of claim 1, wherein Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser, Gly, Thr, or Ile;
Xaa⁷ is Asp or D-Asp;
Xaa⁸ is selected from the group consisting of: Thr, Val, Ile, Leu, hLeu and Nle;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Pge(4-carbomyl), Phe(3-Carbomyl), Tyr(Me), HomoPhe, N-Me-Phe, N-Me-Tyr, Sar, Dihydro Trp, Ile, Leu, Ser, Arg, Thr, Sar, Bpa, Phe(3-Me), Phe(2-Me), Phe(2-CF3), β-Me-Phe, and Ser;
Xaa¹² is selected from the group consisting of: any aromatic amino acid, Glu, D-Glu, homoGlu, Asp, D-Asp, D-homoGlu, Gla, beta-homo-Glu, N-Me-Glu, N-Me-Asp, alpha-H-Glu, corresponding D-amino acid and isosteres;
Xaa¹³ is absent or any amino acid; and
Xaa¹⁴ is any amino acid.

5. The method of claim 1, wherein:
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Pge(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Bpa, Phe(3-Me), Phe(2-Me), Phe(2-CF3), β-Me-Phe, and Ser;
Xaa¹² is absent or selected from the group consisting of: any aromatic amino acid, Glu, D-Glu, homoGlu, Asp, D-Asp, D-homoGlu, Gla, beta-homo-Glu, N-Me-Glu, N-Me-Asp, alpha-H-Glu, corresponding D-amino acid and isosteres;
Xaa¹³ is absent; and
Xaa¹⁴ is any amino acid.

6. The method of claim 1, wherein:
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is selected from the group consisting of: Leu, Nle, Cpa, Cba, HomoLeu, Aoc, and N-Me-Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Pge(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Bpa, Phe(3-Me), Phe(2-Me), Phe(2-CF3), β-Me-Phe, and Ser;
Xaa¹² is absent or selected from the group consisting of: any aromatic amino acid, Glu, D-Glu, beta-homo-Glu, N-Me-Glu, N-Me-Asp, alpha-H-Glu;
Xaa¹³ is absent; and,
Xaa¹⁴ is any amino acid.

7. The method of claim 1, wherein:
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Pge(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Bpa, Phe(3-Me), Phe(2-Me), Phe(2-CF3), β-Me-Phe, and Ser;
Xaa¹² is selected from the group consisting of: any aromatic amino acid, Glu, D-Glu, beta-homo-Glu, N-Me-Glu, N-Me-Asp, alpha-H-Glu, corresponding D-amino acid and isosteres;
Xaa¹³ is absent; and
Xaa¹⁴ is any amino acid.

8. The method of claim 1, wherein:
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp or D-Asp;
Xaa⁸ is Thr or Val;
Xaa⁹ is Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Pge(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Bpa, Phe(3-Me), Phe(2-Me), Phe(2-CF3), β-Me-Phe, and Ser;
Xaa¹² is selected from the group consisting of: any aromatic amino acid, Glu, D-Glu, N-Me-Glu, N-Me-Asp, alpha-H-Glu, and beta-homo-Glu;
Xaa¹³ is absent; and
Xaa¹⁴ is any amino acid.

9. The method of claim 1, wherein:
Xaa¹ is absent or any amino acid;
Xaa² is absent or any amino acid;
Xaa³ is absent or any amino acid;
Xaa⁴ is a 2-methylbenzoyl moiety or a modified HomoSer, optionally Homo-Ser-Cl;
Xaa⁵ is N-Me-Arg;
Xaa⁶ is Ser;
Xaa⁷ is Asp;
Xaa⁸ is Thr or Val;
Xaa⁹ is Leu;
Xaa¹⁰ is Pen, Cys, D-Cys or HomoCys;
Xaa¹¹ is selected from the group consisting of: Trp, Phe, 2-Nal, 1-Nal, Tyr, His, Phe(4-F), Phe(4-CF3), Phe (4-CH3), Phe (4-tBu), Bip, Phe(4-COOH), Gly, 3,3-DiPhenylGly, 3,3 diPhenyl Ala, Tic, b-homo-Trp, D-1-Nal, D-2-Nal, Phe(2,4-diCl), Phe(3,4-diCl), Pge(4-carbomyl), Phe(3-Carbomyl), Tyr(Me), HomoPhe, N-Me-Phe, N-Me-Tyr, Ser, Sar, Dihydro Trp, Ile, Leu, Ser, Arg, Thr, Sar, Bpa, Phe(3-Me), Phe(2-Me), Phe(2-CF3), β-Me-Phe, and Ser;
Xaa¹² is selected from the group consisting of: any aromatic amino acid, Glu, D-Glu, N-Me-Glu, N-Me-Asp, alpha-H-Glu, and beta-homo-Glu;
Xaa¹³ is absent; and
Xaa¹⁴ is any amino acid.

10-36. (canceled)

37. The method of claim 1, wherein the inflammatory bowel disease is ulcerative colitis.

38. The method of claim 1, wherein the inflammatory bowel disease is Crohn's disease.

39. The method of claim 1, wherein the peptide molecule inhibits binding of α4β7 to MAdCAM.

40. The method of claim 1, wherein the peptide molecule or the pharmaceutical composition is provided to the subject in need thereof at an interval sufficient to ameliorate the condition.

41. The method of claim 40, wherein the interval is selected from the group consisting of around the clock, hourly, every four hours, once daily, twice daily, three times daily, four times daily, every other day, weekly, bi-weekly, and monthly.

42. The method of claim 1, wherein the peptide molecule or a pharmaceutical composition thereof is provided as an initial does followed by one or more subsequent doses, and the minimum interval between any two doses is a period of less than 1 day, and wherein each of the doses comprises an effective amount of the peptide molecule.

43. The method of claim 1, wherein the effective amount of the peptide molecule or the pharmaceutical composition is sufficient to achieve at least one of the following: a) about 50% or greater saturation of MAdCAM binding sites on α4β7 integrin molecules; b) about 50% or greater inhibition of α4β7 integrin expression on the cell surface; and c) about 50% or greater saturation of MAdCAM binding sites on α4β7 molecules and about 50% or greater inhibition of α4β7 integrin expression on the cell surface, wherein i) the saturation is maintained for a period consistent with a dosing frequency of no more than twice daily; ii) the inhibition is maintained for a period consistent with a dosing frequency of no more than twice daily; or iii) the saturation and the inhibition are each maintained for a period consistent with a dosing frequency of no more than twice daily.

44. The method of claim 1, wherein the peptide molecule is administered orally.

45. The method of claim 1, wherein the peptide molecule is administered parenterally.

46. The method of claim 1, wherein the peptide molecule is administered topically.

47. The method of claim 1, wherein the peptide molecule comprises the following sequence:
(SEQ ID NO: 270)
2-methylbenzoyl-(N-Me-Arg)-Ser-Asp-Thr-Leu-Pen-
Phe(4-tBu)-(β-homo-Glu)-(D-Lys);


SEQUENCE LISTING (394 sequences)

SEQ ID NO: 1-21 - skipped (21 entries)

SEQ ID NO: 22 - protein, 8 aa, synthetic construct
  REGION 1..8: note = Description of Artificial Sequence: Synthetic peptide
  REGION 1..6: note = CROSSLINK - N-term 2-Me-Benzoyl conjugation with a thioether bond between the N-term and penicillamine
  MOD_RES 1: note = N-Methyl Arginine
  SITE 6: note = Penicillamine
  SITE 8: note = D-Lysine
RSDTLXWK

SEQ ID NO: 23 - protein, 9 aa, synthetic construct
  REGION 1..9: note = Description of Artificial Sequence: Synthetic peptide
  REGION 1..6: note = CROSSLINK - N-term Aromatic acid group, 2-methylbenzoyl, propionyl, isobutyryl, acetyl or butyryl conjugation forming a thioether bond with Xaa at position 6
  MOD_RES 1: note = Optional N(alpha)methylation
  VARIANT 5: note = Leu or Nle
  VARIANT 6: note = Cys, Pen, HCys or D-Cys
  VARIANT 7: note = Trp, Phe, 1-Nal, 2-Nal, D-Phe, Tyr or Phe(CF3)
  VARIANT 8: note = Glu, D-Glu, Beta-HGlu, Tyr or 2-Nal
  VARIANT 9: note = D-Lysine, Lys, Dap, Dab, Leu, Ser, Phe, His, Gln, Tyr, D-Leu, D-Ser, D-Phe, D-Glu, D-Tyr, D-His or Glu
  MOD_RES 9: note = Optional N(alpha)methylation
  MOD_RES 9: note = Optional acetylation
RSDTXXXXX

SEQ ID NO: 24 - protein, 10 aa, synthetic construct
  REGION 1..10: note = Description of Artificial Sequence: Synthetic peptide
  REGION 1..6: note = CROSSLINK - N-term Aromatic acid group, 2-methylbenzoyl, propionyl, isobutyryl, acetyl or butyryl conjugation forming a thioether bond with Xaa at position 6
  MOD_RES 1: note = Optional N(alpha)methylation
  VARIANT 5: note = Any naturally occurring amino acid
  VARIANT 6: note = Cys, Pen, HCys or D-Cys
  VARIANT 7: note = Trp, Phe, 1-Nal, 2-Nal, D-Phe, Tyr or Phe(CF3)
  VARIANT 8: note = Glu, D-Glu, Beta-HGlu, Tyr or 2-Nal
  VARIANT 9: note = Leu, Ser, Phe, His, Gln, Tyr, D-Leu, D-Ser, D-Phe, D-Val, D-Glu, D-Tyr, D-His, D-Glu, Glu, Asp or D-Asp
  VARIANT 10: note = D-Lysine, Lys, Dap, Dab, Leu, Ser, Phe, His, Gln, Tyr, D-Leu, D-Ser, D-Phe, D-Glu, D-Tyr, D-His or Glu
  MOD_RES 10: note = Optional N(alpha)methylation
  MOD_RES 10: note = Optional acetylation
RSDTXXXXXX

SEQ ID NO: 25-46 - skipped (22 entries)

SEQ ID NO: 47 - protein, 9 aa, synthetic construct
  REGION 1..9: note = Description of Artificial Sequence: Synthetic peptide
  REGION 1..6: note = CROSSLINK - N-term 2-methylbenzoyl forming a thioether bond between the N-term and position 6
  MOD_RES 1: note = N-Methyl Arginine
  VARIANT 5: note = Leu or Nle
  VARIANT 6: note = Pen, Cys, homoCys, Pen(=O) or D-Cys
  VARIANT 7: note = Phe, D-Phe, Tyr, D-Tyr, His, Bip, Tic, 1-Nal, 2-Nal, Phe(CH3), Phe(2,4-diCl), Phe(3,4-diCl), Aic, N-Me-Tyr, N-Me-Phe, Phe(2-carbomyl), Phe(3-carbomyl), Phe(4-COOH), Phe(4OMe), Phe(4tBu), Phe-(4-F), Phe(4CF3) or Trp
  VARIANT 8: note = Absent, Glu, Beta-homo-Glu, Bip, O-Me-Glu, D-Lysine, D-Phe, Tyr, 2-Nal, D-Tyr, Pro, Tic, D-Glu, D-Thr, D-Arg, D-Leu, D-Trp, Phe(4-COOH), D-His, Pro, D-Pro or Glu(OMe)
  VARIANT 9: note = Absent or any amino acid residue
RSDTXXXXX

SEQ ID NO: 48-50 - skipped (3 entries)

SEQ ID NO: 51 - protein, 8 aa, synthetic construct
  REGION 1..8: note = Description of Artificial Sequence: Synthetic peptide
  SITE 1: note = N-term Propionyl conjugation
  REGION 1..6: note = CROSSLINK - Thioether linkage between N-term and residue at position 6
  MOD_RES 1: note = N-Methyl Arginine
  SITE 8: note = D-Lysine
  SITE 8: note = C-term NH2
RSDTLCWK

SEQ ID NO: 52 - protein, 8 aa, synthetic construct
  REGION 1..8: note = Description of Artificial Sequence: Synthetic peptide
  REGION 1..6: note = CROSSLINK - N-term Alpha-bromoispbutyryl with a thioether bond between the N-term and the residue at position 6
  MOD_RES 1: note = N-Methyl Arginine
  SITE 8: note = D-Lysine
  SITE 8: note = C-term NH2
RSDTLCWK

SEQ ID NO: 53 - protein, 8 aa, synthetic construct
  REGION 1..8: note = Description of Artificial Sequence: Synthetic peptide
  REGION 1..6: note = CROSSLINK - N-term Acetyl with a thioether bond between the N-term and the residue at position 6
  MOD_RES 1: note = N-Methyl Arginine
  SITE 6: note = Penicillamine
  SITE 8: note = D-Lysine
  SITE 8: note = C-term NH2
RSDTLXWK

SEQ ID NO: 54 - protein, 8 aa, synthetic construct
  REGION 1..8: note = Description of Artificial Sequence: Synthetic peptide
  REGION 1..6: note = CROSSLINK - N-term Propionyl with a thioether bond between the N-term and the residue at position 6
  MOD_RES 1: note = N-Methyl Arginine
  SITE 6: note = Penicillamine
  SITE 8: note = D-Lysine
  SITE 8: note = C-term NH2
RSDTLXWK

SEQ ID NO: 55 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  MOD_RES 9: note = D-Lysine
  SITE 9: note = C-term NH2
RSDTLCWEK

SEQ ID NO: 56 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = D-Lysine
  SITE 9: note = C-term NH2
RSDTLXWEK

SEQ ID NO: 57 - protein, 8 aa, synthetic construct
  REGION 1..8: note = Description of Artificial Sequence: Synthetic peptide
  REGION 1..6: note = CROSSLINK - N-term Propionyl with a thioether bond between the N-term and the residue at position 6
  MOD_RES 1: note = N-Methyl Arginine
  MOD_RES 6: note = Homo-Cysteine
  SITE 8: note = D-Lysine
  SITE 8: note = C-term NH2
RSDTLCWK

SEQ ID NO: 58 - protein, 8 aa, synthetic construct
  REGION 1..8: note = Description of Artificial Sequence: Synthetic peptide
  REGION 1..6: note = CROSSLINK - N-term Butyryl with a thioether bond between the N-term and the residue at position 6
  MOD_RES 1: note = N-Methyl Arginine
  SITE 8: note = D-Lysine
  SITE 8: note = C-term NH2
RSDTLCWK

SEQ ID NO: 59 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  SITE 8: note = D-Lysine
  SITE 8: note = C-term NH2
RSDTLCWK

SEQ ID NO: 60 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 8: note = D-Glu
  MOD_RES 9: note = D-Lysine
  SITE 9: note = C-term NH2
RSDTLXWEK

SEQ ID NO: 61 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 8: note = Beta-Homo-Glu
  SITE 9: note = D-Lysine
  SITE 9: note = C-term NH2
RSDTLXWEK

SEQ ID NO: 62 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 9: note = D Form N-Methyl Lysine
  SITE 9: note = C-term NH2
RSDTLXWEK

SEQ ID NO: 63 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 9: note = N-Methyl Lysine
  SITE 9: note = C-term NH2
RSDTLXWYK

SEQ ID NO: 64 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 5: note = Nle
  SITE 6: note = Penicillamine
  SITE 9: note = D-Lysine
  SITE 9: note = C-term NH2
RSDTXXWEK

SEQ ID NO: 65 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lysine
  SITE 9: note = C-term NH2
RSDTLXFEK

SEQ ID NO: 66 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = D-Cys
  SITE 8: note = Beta-Homo-Glutamic acid
  SITE 9: note = D-Lysine
  SITE 9: note = C-term NH2
RSDTLCWEK

SEQ ID NO: 67 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Homo-Cysteine
  SITE 9: note = D-Lysine
  SITE 9: note = C-term NH2
RSDTLCWEK

SEQ ID NO: 68 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = 1-Napthylalanine
  SITE 8: note = D-Glu
  SITE 9: note = D-Lysine
  SITE 9: note = C-term NH2
RSDTLXXEK

SEQ ID NO: 69 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = D-Glu
  MOD_RES 9: note = N-Methyl Lysine
  SITE 9: note = C-term NH2
RSDTLXXEK

SEQ ID NO: 70 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = Beta-Homo-Glutamic acid
  MOD_RES 9: note = D-Lysine
  SITE 9: note = C-term NH2
RSDTLXXXK

SEQ ID NO: 71 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = D-Phe
  SITE 8: note = 2-Napthylalanine
  SITE 9: note = D-Lysine
  SITE 9: note = C-term NH2
RSDTLXFXK

SEQ ID NO: 72 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = D-Phe
  SITE 9: note = D-Lysine
  SITE 9: note = C-term NH2
RSDTLXFEK

SEQ ID NO: 73 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = Beta-Homo-Glutamic acid
  SITE 9: note = D-Lysine
  SITE 9: note = C-term NH2
RSDTLXFXK

SEQ ID NO: 74 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = Beta-Homo-Glutamic acid
  MOD_RES 9: note = D-Lysine
  SITE 9: note = C-term NH2
RSDTLXYXK

SEQ ID NO: 75 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Glu
  SITE 9: note = D-Lysine
  SITE 9: note = C-term NH2
RSDTLXXEK

SEQ ID NO: 76 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(CF3)
  SITE 9: note = D-Lysine
  SITE 9: note = C-term NH2
RSDTLXFEK

SEQ ID NO: 77 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 9: note = D-Lysine
  SITE 9: note = C-term NH2
RSDTLXXEK

SEQ ID NO: 78 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = D-Lysine
  SITE 9: note = C-term NH2
RSDTLXYEK

SEQ ID NO: 79 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lysine
  SITE 9: note = C-term NH2
RSDTLXYEK

SEQ ID NO: 80 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = Acetylated D-Lysine
  SITE 9: note = C-term NH2
RSDTLXWEK

SEQ ID NO: 81 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = Acetylated D-Lysine
  SITE 9: note = C-term NH2
RSDTLXWEK

SEQ ID NO: 82 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lysine(PEG8)
  SITE 9: note = PEG8 conjugation
  SITE 9: note = C-term NH2
RSDTLXWEK

SEQ ID NO: 83 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Pen
  SITE 8: note = Beta-Homo-Glutamic acid
  SITE 9: note = Acetylated D-Lysine
  SITE 9: note = C-term NH2
RSDTLXWXK

SEQ ID NO: 84 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 9: note = Acetylated D Form N-Methyl Lysine
  SITE 9: note = C-term NH2
RSDTLXWEK

SEQ ID NO: 85 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 9: note = Acetylated N-Methyl Lysine
  SITE 9: note = C-term NH2
RSDTLXWYK

SEQ ID NO: 86 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 5: note = Nle
  SITE 6: note = Penicillamine
  MOD_RES 9: note = Acetylated D-Form N-Methyl Lysine
  SITE 9: note = C-term NH2
RSDTXXWEK

SEQ ID NO: 87 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  MOD_RES 9: note = Acetylated D-Form N-Methyl Lysine
  SITE 9: note = C-term NH2
RSDTLXFEK

SEQ ID NO: 88 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Trifluoromethyl Phenylalanine
  SITE 9: note = Acetylated D-Form N-Methyl Lysine
  SITE 9: note = C-term NH2
RSDTLXFEK

SEQ ID NO: 89 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 9: note = Acetylated D-Form Lysine
  SITE 9: note = C-term NH2
RSDTLXXEK

SEQ ID NO: 90 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = Acetylated D-Form Lysine
  SITE 9: note = C-term NH2
RSDTLXYEK

SEQ ID NO: 91 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = Acetylated D-Form Lysine
  SITE 9: note = C-term NH2
RSDTLXYEK

SEQ ID NO: 92 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = Diaminopropionic acid
  SITE 9: note = C-term NH2
RSDTLXWEX

SEQ ID NO: 93 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = Diaminobutyric acid
  SITE 9: note = C-term NH2
RSDTLXWEX

SEQ ID NO: 94 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = Diaminopropionic acid
  SITE 9: note = C-term NH2
RSDTLXWEX

SEQ ID NO: 95 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = Diaminobutyric acid
  SITE 9: note = C-term NH2
RSDTLXWEX

SEQ ID NO: 96 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = C-term NH2
RSDTLXWE

SEQ ID NO: 97 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 8: note = C-term NH2
RSDTLXWE

SEQ ID NO: 98 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 8: note = C-term NH2
RSDTLXWE

SEQ ID NO: 99 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = C-term NH2
RSDTLXWEL

SEQ ID NO: 100 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = C-term NH2
RSDTLXWES

SEQ ID NO: 101 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = C-term NH2
RSDTLXWEF

SEQ ID NO: 102 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = C-term NH2
RSDTLXWEH

SEQ ID NO: 103 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = C-term NH2
RSDTLXWEQ

SEQ ID NO: 104 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = C-term NH2
RSDTLXWEY

SEQ ID NO: 105 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Leu
  SITE 9: note = C-term NH2
RSDTLXWEL

SEQ ID NO: 106 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Ser
  SITE 9: note = C-term NH2
RSDTLXWES

SEQ ID NO: 107 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Phe
  SITE 9: note = C-term NH2
RSDTLXWEF

SEQ ID NO: 108 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Glu
  SITE 9: note = C-term NH2
RSDTLXWEE

SEQ ID NO: 109 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-His
  SITE 9: note = C-term NH2
RSDTLXWEH

SEQ ID NO: 110 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Tyr
  SITE 9: note = C-term NH2
RSDTLXWEY

SEQ ID NO: 111 - protein, 9 aa, synthetic construct
  REGION 1..9: note = Description of Artificial Sequence: Synthetic peptide
  REGION 1..6: note = CROSSLINK - N-term 3-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 112 - protein, 9 aa, synthetic construct
  REGION 1..9: note = Description of Artificial Sequence: Synthetic peptide
  REGION 1..6: note = CROSSLINK - N-term 4-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 113 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = C-term NH2
RSDTLXWEE

SEQ ID NO: 114 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Glu
  SITE 8: note = C-term NH2
RSDTLXXE

SEQ ID NO: 115 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 8: note = L-glutamic acid g-methyl ester
  SITE 9: note = D-Lys
  SITE 9: note = C-term NH2
RSDTLXWEK

SEQ ID NO: 116 - protein, 7 aa, synthetic construct
  REGION 1..7: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 7: note = C-term NH2
RSDTLXX

SEQ ID NO: 117 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 9: note = D-Lys
  SITE 9: note = C-term NH2
RSDTLCXEK

SEQ ID NO: 118 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 8: note = D-Lys
  SITE 8: note = C-term OH
RSDTLCXK

SEQ ID NO: 119 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Atc
  SITE 8: note = Beta-Homo-Glutamic acid
  SITE 8: note = C-term NH2
RSDTLXXE

SEQ ID NO: 120 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = erythro-Beta-phenyl-Ser
  SITE 8: note = Beta-Homo-Glutamic acid
  SITE 8: note = C-term NH2
RSDTLXSE

SEQ ID NO: 121 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = erythro-Beta-phenyl-Ser
  SITE 8: note = Beta-Homo-Glutamic acid
  SITE 8: note = C-term NH2
RSDTLXSE

SEQ ID NO: 122 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = threo-Beta-phenyl-Ser
  SITE 8: note = Beta-Homo-Glutamic acid
  SITE 8: note = C-term NH2
RSDTLXSE

SEQ ID NO: 123 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = threo-Beta-phenyl-Ser
  SITE 8: note = Beta-Homo-Glutamic acid
  SITE 8: note = C-term NH2
RSDTLXSE

SEQ ID NO: 124 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = benzoyl-L-phenylalanine
  SITE 8: note = Beta-Homo-Glutamic acid
  SITE 8: note = C-term NH2
RSDTLXXE

SEQ ID NO: 125 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = 3-Methyl Phenylalanine
  SITE 8: note = Beta-Homo-Glutamic acid
  SITE 8: note = C-term NH2
RSDTLXFE

SEQ ID NO: 126 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = 2-Methyl Phenylalanine
  SITE 8: note = Beta-Homo-Glutamic acid
  SITE 8: note = C-term NH2
RSDTLXFE

SEQ ID NO: 127 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Trifluoromethyl Phenylalanine
  SITE 8: note = Beta-Homo-Glutamic acid
  SITE 8: note = C-term NH2
RSDTLXFE

SEQ ID NO: 128 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Beta Methyl Phenylalanine
  SITE 8: note = Beta-Homo-Glutamic acid
  SITE 8: note = C-term NH2
RSDTLXFE

SEQ ID NO: 129 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Beta Methyl Phenylalanine
  SITE 8: note = Beta-Homo-Glutamic acid
  SITE 8: note = C-term NH2
RSDTLXFE

SEQ ID NO: 130 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Beta Dimethyl Phenylalanine
  SITE 8: note = Beta-Homo-Glutamic acid
  SITE 8: note = C-term NH2
RSDTLXFE

SEQ ID NO: 131 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Beta Dimethyl Phenylalanine
  SITE 8: note = Beta-Homo-Glutamic acid
  SITE 8: note = C-term NH2
RSDTLXFE

SEQ ID NO: 132 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = 4-methyl Phenylalanine
  SITE 8: note = Beta-Homo-Glutamic acid
  SITE 8: note = C-term NH2
RSDTLXFE

SEQ ID NO: 133 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 8: note = Beta-Homo-Glutamic acid
  SITE 8: note = C-term NH2
RSDTLXXE

SEQ ID NO: 134 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = Beta-Homo-Glutamic acid
  SITE 8: note = C-term NH2
RSDTLXFE

SEQ ID NO: 135 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  MOD_RES 8: note = N-Methyl Glutamic Acid
  SITE 8: note = C-term NH2
RSDTLXFE

SEQ ID NO: 136 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  MOD_RES 8: note = N-Methyl Aspartic Acid
  SITE 8: note = C-term NH2
RSDTLXFD

SEQ ID NO: 137 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = alpha-Homo-Glutamic acid
  SITE 8: note = C-term NH2
RSDTLXXX

SEQ ID NO: 138 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  SITE 1: note = Citroline
  SITE 6: note = Penicillamine
  SITE 7: note = (S) -2-amino-3-(4-tert-butyl-phenyl)propionic acid
  SITE 8: note = Beta-Homo-Glutamic acid
  SITE 8: note = C-term NH2
XSDTLXXX

SEQ ID NO: 139 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = Beta-Homo-Glutamic acid
  SITE 8: note = C-term NH2
RADTLXXX

SEQ ID NO: 140 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 2: note = Aminobutyric acid
  SITE 6: note = Penicillamine
  SITE 7: note = (S) -2-amino-3-(4-tert-butyl-phenyl)propionic acid
  SITE 8: note = Beta-Homo-Glutamic acid
  SITE 8: note = C-term NH2
RXDTLXXX

SEQ ID NO: 141 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 2: note = Tbu
  SITE 6: note = Penicillamine
  SITE 7: note = (S) -2-amino-3-(4-tert-butyl-phenyl)propionic acid
  SITE 8: note = Beta-Homo-Glutamic acid
  SITE 8: note = C-term NH2
RXDTLXXX

SEQ ID NO: 142 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  MOD_RES 8: note = N-Methyl Glutamic Acid
  SITE 8: note = C-term NH2
RSDTLXXE

SEQ ID NO: 143 - protein, 8 aa, synthetic construct
  REGION 1..8: note = Description of Artificial Sequence: Synthetic peptide
  REGION 1..6: note = CROSSLINK - N-term Acetyl with a thioether bond between the N-term and the residue at position 6
  MOD_RES 1: note = N-Methyl Arginine
  SITE 8: note = D-Lys
  SITE 8: note = C-term NH2
RSDTLCWK

SEQ ID NO: 144 - protein, 8 aa, synthetic construct
  REGION 1..8: note = Description of Artificial Sequence: Synthetic peptide
  REGION 1..6: note = CROSSLINK - N-term Propionyl with a thioether bond between the N-term and the residue at position 6
  MOD_RES 1: note = N-Methyl Arginine
  SITE 8: note = D-Lys
  SITE 8: note = C-term NH2
RSDTLCWK

SEQ ID NO: 145 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 9: note = D-Lys
  SITE 9: note = C-term NH2
RSDTLCWEK

SEQ ID NO: 146 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = D-Lys
  SITE 9: note = C-term NH2
RSDTLXWEK

SEQ ID NO: 147 - protein, 8 aa, synthetic construct
  REGION 1..8: note = Description of Artificial Sequence: Synthetic peptide
  REGION 1..6: note = CROSSLINK - N-term Acetyl with a thioether bond between the N-term and the residue at position 6
  MOD_RES 1: note = N-Methyl Arginine
  SITE 6: note = Penicillamine
  SITE 8: note = D-Lys
  SITE 8: note = C-term NH2
RSDTLXWK

SEQ ID NO: 148 - protein, 8 aa, synthetic construct
  REGION 1..8: note = Description of Artificial Sequence: Synthetic peptide
  REGION 1..6: note = CROSSLINK - N-term Propionyl with a thioether bond between the N-term and the residue at position 6
  MOD_RES 1: note = N-Methyl Arginine
  SITE 6: note = Penicillamine
  SITE 8: note = D-Lys
  SITE 8: note = C-term NH2
RSDTLXWK

SEQ ID NO: 149 - protein, 8 aa, synthetic construct
  REGION 1..8: note = Description of Artificial Sequence: Synthetic peptide
  REGION 1..6: note = CROSSLINK - N-term Propionyl with a thioether bond between the N-term and the residue at position 6
  MOD_RES 1: note = N-Methyl Arginine
  SITE 8: note = homo-Cysteine
  SITE 8: note = D-Lys
  SITE 8: note = C-term NH2
RSDTLXWK

SEQ ID NO: 150 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 151 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = Beta-Homo-Glutamic acid
  SITE 9: note = D-Lys
  SITE 9: note = C-term NH2
RSDTLXWXK

SEQ ID NO: 152 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = D-Form N-Methyl Lysine
  SITE 9: note = C-term NH2
RSDTLXWEK

SEQ ID NO: 153 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 9: note = N-Methyl Lysine
  SITE 9: note = C-term NH2
RSDTLXWYK

SEQ ID NO: 154 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 5: note = Nle
  SITE 6: note = Penicillamine
  SITE 9: note = D-Lys
  SITE 9: note = C-term NH2
RSDTXXWEK

SEQ ID NO: 155 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXFEK

SEQ ID NO: 156 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = D-Cys
  SITE 8: note = Beta-Homo-Glutamic acid
  SITE 9: note = D-Lys
  SITE 9: note = C-term NH2
RSDTLCWEK

SEQ ID NO: 157 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 8: note = homo-Cysteine
  SITE 9: note = D-Lys
  SITE 9: note = C-term NH2
RSDTLXWEK

SEQ ID NO: 158 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = D-Glu
  SITE 9: note = D-Lys
  SITE 9: note = C-term NH2
RSDTLXXEK

SEQ ID NO: 159 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Glu
  SITE 9: note = D-Lys
  SITE 9: note = C-term NH2
RSDTLXXEK

SEQ ID NO: 160 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Glu
  MOD_RES 9: note = N-Methyl Lysine
  SITE 9: note = C-term NH2
RSDTLXXEK

SEQ ID NO: 161 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = Beta-Homo-Glutamic acid
  SITE 9: note = D-Lys
  SITE 9: note = C-term NH2
RSDTLXXXK

SEQ ID NO: 162 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = D-Phe
  SITE 8: note = 2-Naphthylalanine
  SITE 9: note = D-Lys
  SITE 9: note = C-term NH2
RSDTLXFXK

SEQ ID NO: 163 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = D-Phe
  SITE 9: note = D-Lys
  SITE 9: note = C-term NH2
RSDTLXFEK

SEQ ID NO: 164 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = Beta-Homo-Glutamic acid
  MOD_RES 9: note = D-Lysine
  SITE 9: note = C-term NH2
RSDTLXFXK

SEQ ID NO: 165 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = Beta-Homo-Glutamic acid
  SITE 9: note = D-Lys
  SITE 9: note = C-term NH2
RSDTLXYXK

SEQ ID NO: 166 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(CF3)
  SITE 9: note = D-Lys
  SITE 9: note = C-term NH2
RSDTLXFEK

SEQ ID NO: 167 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 9: note = D-Lys
  SITE 9: note = C-term NH2
RSDTLXXEK

SEQ ID NO: 168 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = D-Lys
  SITE 9: note = C-term NH2
RSDTLXYEK

SEQ ID NO: 169 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXYEK

SEQ ID NO: 170 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 171 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 172 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine(=O)
  SITE 7: note = 2-Napthylalanine
  SITE 8: note = D-Glu
  SITE 9: note = D-Lys
  SITE 9: note = C-term NH2
RSDTLXXEK

SEQ ID NO: 173 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine(=O)
  SITE 7: note = 2-Napthylalanine
  SITE 8: note = D-Glu
  SITE 9: note = D-Lys
  SITE 9: note = C-term NH2
RSDTLXXEK

SEQ ID NO: 174 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 175 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 176 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S)- 2-amino-3-(2,4-dichlorophenyl)propionic acid
  SITE 8: note = D-Glu
  SITE 9: note = D-Lys
  SITE 9: note = C-term NH2
RSDTLXXEK

SEQ ID NO: 177 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S)- 2-amino-3-(3,4-dichlorophenyl)propionic acid
  SITE 8: note = D-Glu
  SITE 9: note = D-Lys
  SITE 9: note = C-term NH2
RSDTLXXEK

SEQ ID NO: 178 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 8: note = D-Glu
  SITE 9: note = D-Lys
  SITE 9: note = C-term NH2
RSDTLXXEK

SEQ ID NO: 179 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = D-Cys
  SITE 7: note = aminoindan-2-carboxylic acid
  SITE 8: note = D-Glu
  SITE 9: note = D-Lys
  SITE 9: note = C-term NH2
RSDTLCXEK

SEQ ID NO: 180 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = aminoindan-2-carboxylic acid
  SITE 8: note = D-Glu
  SITE 9: note = D-Lys
  SITE 9: note = C-term NH2
RSDTLCXEK

SEQ ID NO: 181 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = D-Penicillamine
  SITE 9: note = D-Lys
  SITE 9: note = C-term NH2
RSDTLXWEK

SEQ ID NO: 182 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  MOD_RES 7: note = N-Methyl Tyrosine
  SITE 9: note = D-Lys
  SITE 9: note = C-term NH2
RSDTLCYEK

SEQ ID NO: 183 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  MOD_RES 7: note = N-Methyl Phenylalanine
  SITE 9: note = D-Lys
  SITE 9: note = C-term NH2
RSDTLCFEK

SEQ ID NO: 184 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 8: note = D-Glu
  SITE 9: note = D-Lys
  SITE 9: note = C-term NH2
RSDTLCXEK

SEQ ID NO: 185 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = D-Cys
  SITE 7: note = (3S-)-1,2,3,4-tetrahydroisoquinoline-3-carboxylic acid
  SITE 8: note = D-Glu
  SITE 9: note = D-Lys
  SITE 9: note = C-term NH2
RSDTLCXEK

SEQ ID NO: 186 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = D-Phe
  SITE 9: note = D-Lys
  SITE 9: note = C-term NH2
RSDTLCFEK

SEQ ID NO: 187 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = D-Phe
  SITE 8: note = D-Glu
  SITE 9: note = D-Lys
  SITE 9: note = C-term NH2
RSDTLCFEK

SEQ ID NO: 188 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = D-Penicillamine
  SITE 8: note = D-Glu
  SITE 9: note = D-Lys
  SITE 9: note = C-term NH2
RSDTLXYEK

SEQ ID NO: 189 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSETLXFEK

SEQ ID NO: 190 - protein, 10 aa, synthetic construct
  REGION 1..10: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 10: note = D-Lysine
  SITE 10: note = C-term NH2
RSDTLXWELK

SEQ ID NO: 191 - protein, 10 aa, synthetic construct
  REGION 1..10: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 10: note = D-Lysine
  SITE 10: note = C-term NH2
RSDTLXWESK

SEQ ID NO: 192 - protein, 10 aa, synthetic construct
  REGION 1..10: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 10: note = D-Lysine
  SITE 10: note = C-term NH2
RSDTLXWEFK

SEQ ID NO: 193 - protein, 10 aa, synthetic construct
  REGION 1..10: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 10: note = D-Lysine
  SITE 10: note = C-term NH2
RSDTLXWEHK

SEQ ID NO: 194 - protein, 10 aa, synthetic construct
  REGION 1..10: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 10: note = D-Lysine
  SITE 10: note = C-term NH2
RSDTLXWEEK

SEQ ID NO: 195 - protein, 10 aa, synthetic construct
  REGION 1..10: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 10: note = D-Lysine
  SITE 10: note = C-term NH2
RSDTLXWEYK

SEQ ID NO: 196 - protein, 10 aa, synthetic construct
  REGION 1..10: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Leu
  SITE 10: note = D-Lysine
  SITE 10: note = C-term NH2
RSDTLXWELK

SEQ ID NO: 197 - protein, 10 aa, synthetic construct
  REGION 1..10: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Ser
  SITE 10: note = D-Lysine
  SITE 10: note = C-term NH2
RSDTLXWESK

SEQ ID NO: 198 - protein, 10 aa, synthetic construct
  REGION 1..10: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Phe
  SITE 10: note = D-Lysine
  SITE 10: note = C-term NH2
RSDTLXWEFK

SEQ ID NO: 199 - protein, 10 aa, synthetic construct
  REGION 1..10: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-His
  SITE 10: note = D-Lysine
  SITE 10: note = C-term NH2
RSDTLXWEHK

SEQ ID NO: 200 - protein, 10 aa, synthetic construct
  REGION 1..10: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Glu
  SITE 10: note = D-Lysine
  SITE 10: note = C-term NH2
RSDTLXWEEK

SEQ ID NO: 201 - protein, 10 aa, synthetic construct
  REGION 1..10: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Tyr
  SITE 10: note = D-Lysine
  SITE 10: note = C-term NH2
RSDTLXWEYK

SEQ ID NO: 202 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = Biphenylalanine
  SITE 9: note = D-Lys
  SITE 9: note = C-term NH2
RSDTLXWXK

SEQ ID NO: 203 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = Biphenylalanine
  SITE 9: note = D-Lys
  SITE 9: note = C-term NH2
RSDTLXFXK

SEQ ID NO: 204 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXFEK

SEQ ID NO: 205 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 8: note = Biphenylalanine
  SITE 9: note = D-Lys
  SITE 9: note = C-term NH2
RSDTLCXXK

SEQ ID NO: 206 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = Biphenylalanine
  SITE 9: note = D-Lys
  SITE 9: note = C-term NH2
RSDTLXXXK

SEQ ID NO: 207 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 8: note = D-Glu
  SITE 9: note = D-Lys
  SITE 9: note = C-term NH2
RSDTLCXEK

SEQ ID NO: 208 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 8: note = D-Glu
  SITE 9: note = D-Lys
  SITE 9: note = C-term NH2
RSDTLXXEK

SEQ ID NO: 209 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 210 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 8: note = L-glutamic acid g-methyl ester
  SITE 9: note = D-Lys
  SITE 9: note = C-term NH2
RSDTLXWEK

SEQ ID NO: 211 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  MOD_RES 8: note = L-glutamic acid g-methyl ester
  SITE 9: note = D-Lys
  SITE 9: note = C-term NH2
RSDTLCXEK

SEQ ID NO: 212 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 213 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 214 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 215 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 216 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 8: note = D-Lys
  SITE 8: note = C-term NH2
RSDTLCXK

SEQ ID NO: 217 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 218 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 219 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 9: note = D-Lys
  SITE 9: note = C-term NH2
RSDTLCXEK

SEQ ID NO: 220 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  MOD_RES 8: note = L-glutamic acid g-methyl ester
  SITE 9: note = D-Lys
  SITE 9: note = C-term NH2
RSDTLCXEK

SEQ ID NO: 221 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 8: note = D-Lys
  SITE 8: note = C-term NH2
RSDTLCXK

SEQ ID NO: 222 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = Beta-Homo-Glutamic acid
  SITE 9: note = D-Lys
  SITE 9: note = C-term NH2
RSDTLXXXK

SEQ ID NO: 223 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = Beta-Homo-Glutamic acid
  SITE 9: note = D-Lys
  SITE 9: note = C-term NH2
RSDTLXXXK

SEQ ID NO: 224 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = Diaminopropionic acid
  SITE 9: note = C-term Ac
RSDTLXWEX

SEQ ID NO: 225 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 5: note = Nle
  SITE 6: note = Penicillamine
  SITE 8: note = D-Glu
  SITE 9: note = D-Form N-Methyl Lysine
  SITE 9: note = C-term NH2
RSDTXXFEK

SEQ ID NO: 226 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 5: note = Nle
  SITE 6: note = Penicillamine
  MOD_RES 9: note = N-Methyl Lysine
  SITE 9: note = C-term NH2
RSDTXXWEK

SEQ ID NO: 227 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 5: note = Nle
  SITE 6: note = Penicillamine
  SITE 8: note = D-Glu
  SITE 9: note = D-Form N-Methyl Lysine
  SITE 9: note = C-term NH2
RSDTXXFEK

SEQ ID NO: 228 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 5: note = Nle
  SITE 6: note = Penicillamine
  SITE 9: note = D-Form N-Methyl Lysine
  SITE 9: note = C-term NH2
RSDTXXWEK

SEQ ID NO: 229 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 5: note = Nle
  SITE 6: note = Penicillamine
  SITE 8: note = D-Glu
  SITE 9: note = D-Form N-Methyl Lysine
  SITE 9: note = C-term NH2
RSDTXXFEK

SEQ ID NO: 230 - protein, 5 aa, synthetic construct
  REGION 1..5: note = Description of Artificial Sequence: Synthetic peptide
  REGION 1..5: note = CROSSLINK - N-term C(thioether propane) with a thioether bond between the N-term and the C-term
  MOD_RES 1: note = N-Methyl Arginine
  SITE 5: note = C-term C(thioether propane)
RSDTL

SEQ ID NO: 231 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = Diaminobutyric acid
  SITE 9: note = C-term Ac
RSDTLXWEX

SEQ ID NO: 232 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = Diaminobutyric acid
  SITE 9: note = C-term Ac
RSDTLXWEX

SEQ ID NO: 233 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = Diaminobutyric acid
  SITE 9: note = C-term NH2
RSDTLXWEX

SEQ ID NO: 234 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = Diaminobutyric acid
  REGION 1..9: note = C-term Ac
RSDTLXWEX

SEQ ID NO: 235 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 8: note = D-Glu
  SITE 9: note = D-Lys
  SITE 9: note = C-term NH2
RSDTLCXEK

SEQ ID NO: 236 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Phe
  SITE 9: note = D-Lys
  SITE 9: note = C-term NH2
RSDTLXWFK

SEQ ID NO: 237 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Tyr
  SITE 9: note = D-Lys
  SITE 9: note = C-term NH2
RSDTLXWYK

SEQ ID NO: 238 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 8: note = D-Glu
  SITE 9: note = D-Lys
  SITE 9: note = C-term NH2
RSDTLCXEK

SEQ ID NO: 239 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = D-Lys
  SITE 9: note = C-term NH2
RSDTLXWPK

SEQ ID NO: 240 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = C-term NH2
RSDTLXWPK

SEQ ID NO: 241 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Pro
  SITE 9: note = C-term NH2
RSDTLXWPK

SEQ ID NO: 242 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = L-2-carbamoylphenylalanine
  SITE 8: note = D-Glu
  SITE 9: note = D-Lys
  SITE 9: note = C-term NH2
RSDTLXXEK

SEQ ID NO: 243 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = L-3-carbamoylphenylalanine
  SITE 8: note = D-Glu
  SITE 9: note = D-Lys
  SITE 9: note = C-term NH2
RSDTLXXEK

SEQ ID NO: 244 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 8: note = D-Glu
  SITE 9: note = D-Lys
  SITE 9: note = C-term NH2
RSDTLXFEK

SEQ ID NO: 245 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S)- 2-amino-3-(2,4-dichlorophenyl)propionic acid
  SITE 8: note = D-Glu
  SITE 9: note = D-Lys
  SITE 9: note = C-term NH2
RSDTLXXEK

SEQ ID NO: 246 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S)- 2-amino-3-(3,4-dichlorophenyl)propionic acid
  SITE 8: note = D-Glu
  SITE 9: note = D-Lys
  SITE 9: note = C-term NH2
RSDTLXXEK

SEQ ID NO: 247 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-OMe)
  SITE 8: note = D-Glu
  SITE 9: note = D-Lys
  SITE 9: note = C-term NH2
RSDTLXFEK

SEQ ID NO: 248 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-His
  SITE 9: note = D-Lys
  SITE 9: note = C-term NH2
RSDTLXWHK

SEQ ID NO: 249 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 8: note = Phe(4-COOH)
  SITE 9: note = D-Lys
  SITE 9: note = C-term NH2
RSDTLXWFK

SEQ ID NO: 250 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = D-Glu
  SITE 9: note = D-Lys
  SITE 9: note = C-term NH2
RSDTLXXEK

SEQ ID NO: 251 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = 4-fluoro-L-phenylalanine
  SITE 8: note = D-Glu
  SITE 9: note = D-Lys
  SITE 9: note = C-term NH2
RSDTLXFEK

SEQ ID NO: 252 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 8: note = D-Glu
  SITE 9: note = D-Lys
  SITE 9: note = C-term NH2
RSDTLXXEK

SEQ ID NO: 253 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = (3S-)-1,2,3,4-tetrahydroisoquinoline-3-carboxylic acid
  SITE 9: note = D-Lys
  SITE 9: note = C-term NH2
RSDTLXWXK

SEQ ID NO: 254 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Trp
  SITE 9: note = D-Lys
  SITE 9: note = C-term NH2
RSDTLXWWK

SEQ ID NO: 255 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = D-Phe
  SITE 9: note = D-Lys
  SITE 9: note = C-term NH2
RSDTLXXFK

SEQ ID NO: 256 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = D-His
  SITE 9: note = D-Lys
  SITE 9: note = C-term NH2
RSDTLXXHK

SEQ ID NO: 257 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = D-Leu
  SITE 9: note = D-Lys
  SITE 9: note = C-term NH2
RSDTLXXLK

SEQ ID NO: 258 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = D-Arg
  SITE 9: note = D-Lys
  SITE 9: note = C-term NH2
RSDTLXXRK

SEQ ID NO: 259 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = (3S-)-1,2,3,4-tetrahydroisoquinoline-3-carboxylic acid
  SITE 9: note = D-Lys
  SITE 9: note = C-term NH2
RSDTLXXXK

SEQ ID NO: 260 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = D-Thr
  SITE 9: note = D-Lys
  SITE 9: note = C-term NH2
RSDTLXXTK

SEQ ID NO: 261 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Phe
  SITE 9: note = D-Lys
  SITE 9: note = C-term NH2
RSDTLXXFK

SEQ ID NO: 262 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-His
  SITE 9: note = D-Lys
  SITE 9: note = C-term NH2
RSDTLXXHK

SEQ ID NO: 263 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Leu
  SITE 9: note = D-Lys
  SITE 9: note = C-term NH2
RSDTLXXLK

SEQ ID NO: 264 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Arg
  SITE 9: note = D-Lys
  SITE 9: note = C-term NH2
RSDTLXXRK

SEQ ID NO: 265 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = (3S-)-1,2,3,4-tetrahydroisoquinoline-3-carboxylic acid
  SITE 9: note = D-Lys
  SITE 9: note = C-term NH2
RSDTLXXXK

SEQ ID NO: 266 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 4-Trifluoro-Phenylalanine
  SITE 8: note = D-Glu
  SITE 9: note = D-Lys
  SITE 9: note = C-term NH2
RSDTLXXEK

SEQ ID NO: 267 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXYEK

SEQ ID NO: 268 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXHEK

SEQ ID NO: 269 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 9: note = D-Lys
  SITE 9: note = C-term NH2
RSDTLXXEK

SEQ ID NO: 270 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = Beta-Homo-Glutamic acid
  SITE 9: note = D-Lys
  SITE 9: note = C-term NH2
RSDTLXXXK

SEQ ID NO: 271 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 9: note = D-Lys
  SITE 9: note = C-term NH2
RSDTLXFEK

SEQ ID NO: 272 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 9: note = D-Lys
  SITE 9: note = C-term NH2
RSDTLXFEK

SEQ ID NO: 273 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 9: note = D-Lys
  SITE 9: note = C-term NH2
RSDTLXFEK

SEQ ID NO: 274 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 8: note = Beta-Homo-Glutamic acid
  SITE 9: note = D-Lys
  SITE 9: note = C-term NH2
RSDTLXFXK

SEQ ID NO: 275 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 8: note = Beta-Homo-Glutamic acid
  SITE 9: note = D-Lys
  SITE 9: note = C-term NH2
RSDTLXFXK

SEQ ID NO: 276 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 8: note = Beta-Homo-Glutamic acid
  SITE 9: note = D-Lys
  SITE 9: note = C-term NH2
RSDTLXFXK

SEQ ID NO: 277 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 9: note = D-Lys
  SITE 9: note = C-term NH2
RSDTLXXEK

SEQ ID NO: 278 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 8: note = Beta-Homo-Glutamic acid
  SITE 9: note = D-Lys
  SITE 9: note = C-term NH2
RSDTLXXXK

SEQ ID NO: 279 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 9: note = D-Lys
  SITE 9: note = C-term NH2
RSDTLXXEK

SEQ ID NO: 280 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = Beta-Homo-Glutamic acid
  SITE 9: note = D-Lys
  SITE 9: note = C-term NH2
RSDTLXXXK

SEQ ID NO: 281 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Naphthylalanine
  SITE 9: note = D-Lys
  SITE 9: note = C-term NH2
RSDTLXXEK

SEQ ID NO: 282 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = Beta-Homo-Glutamic acid
  SITE 9: note = D-Lys
  SITE 9: note = C-term NH2
RSDTLXXXK

SEQ ID NO: 283 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Lys
  SITE 8: note = C-term NH2
RSDTLXXK

SEQ ID NO: 284 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Lys
  SITE 8: note = C-term NH2
RSDTLXXK

SEQ ID NO: 285 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  MOD_RES 9: note = N-Methyl Lysine
  SITE 9: note = C-term NH2
RSDTLXXEK

SEQ ID NO: 286 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  MOD_RES 9: note = D-Form N-Methyl Lysine
  SITE 9: note = C-term NH2
RSDTLXXEK

SEQ ID NO: 287 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = Beta-Homo-Glutamic acid
  MOD_RES 9: note = N-Methyl Lysine
  SITE 9: note = C-term NH2
RSDTLXXXK

SEQ ID NO: 288 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = Beta-Homo-Glutamic acid
  MOD_RES 9: note = D-Form N-Methyl Lysine
  SITE 9: note = C-term NH2
RSDTLXXXK

SEQ ID NO: 289 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  MOD_RES 9: note = N-Methyl Lysine
  SITE 9: note = C-term NH2
RSDTLXXEK

SEQ ID NO: 290 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  MOD_RES 9: note = D-Form N-Methyl Lysine
  SITE 9: note = C-term NH2
RSDTLXXEK

SEQ ID NO: 291 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 8: note = Beta-Homo-Glutamic acid
  MOD_RES 9: note = N-Methyl Lysine
  SITE 9: note = C-term NH2
RSDTLXXXK

SEQ ID NO: 292 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 8: note = Beta-Homo-Glutamic acid
  MOD_RES 9: note = D-Form N-Methyl Lysine
  SITE 9: note = C-term NH2
RSDTLXXXK

SEQ ID NO: 293 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  MOD_RES 9: note = N-Methyl Lysine
  SITE 9: note = C-term NH2
RSDTLXXEK

SEQ ID NO: 294 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  MOD_RES 9: note = D-Form N-Methyl Lysine
  SITE 9: note = C-term NH2
RSDTLXXEK

SEQ ID NO: 295 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = Beta-Homo-Glutamic acid
  MOD_RES 9: note = N-Methyl Lysine
  SITE 9: note = C-term NH2
RSDTLXXXK

SEQ ID NO: 296 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = Beta-Homo-Glutamic acid
  MOD_RES 9: note = D-Form N-Methyl Lysine
  SITE 9: note = C-term NH2
RSDTLXXXK

SEQ ID NO: 297 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  MOD_RES 9: note = N-Methyl Lysine
  SITE 9: note = C-term NH2
RSDTLXXEK

SEQ ID NO: 298 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  MOD_RES 9: note = D-Form N-Methyl Lysine
  SITE 9: note = C-term NH2
RSDTLXXEK

SEQ ID NO: 299 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = Beta-Homo-Glutamic acid
  MOD_RES 9: note = N-Methyl Lysine
  SITE 9: note = C-term NH2
RSDTLXXXK

SEQ ID NO: 300 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = Beta-Homo-Glutamic acid
  MOD_RES 9: note = D-Form N-Methyl Lysine
  SITE 9: note = C-term NH2
RSDTLXXXK

SEQ ID NO: 301 - protein, 8 aa, synthetic construct
  REGION 1..8: note = Description of Artificial Sequence: Synthetic peptide
  REGION 1..6: note = CROSSLINK - N-term Butyryl with a thioether bond between the N-term and the residue at position 6
  MOD_RES 1: note = N-Methyl Arginine
  SITE 8: note = D-Lys
  SITE 8: note = C-term NH2
RSDTLCWK

SEQ ID NO: 302 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = D-Cys
  SITE 8: note = Beta-Homo-Glutamic acid
  SITE 9: note = D-Lys
  SITE 9: note = C-term NH2
RSDTLCWXK

SEQ ID NO: 303 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = homo-Cysteine
  SITE 9: note = D-Lys
  SITE 9: note = C-term NH2
RSDTLXWEK

SEQ ID NO: 304 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = Diaminopropionic acid
  SITE 9: note = C-term Ac
RSDTLXWEX

SEQ ID NO: 305 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = Diaminobutyric acid
  SITE 9: note = C-term Ac
RSDTLXWEX

SEQ ID NO: 306 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = Diaminopropionic acid
  SITE 9: note = C-term Ac
RSDTLXWEX

SEQ ID NO: 307 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = Diaminobutyric acid
  SITE 9: note = C-term Ac
RSDTLXWEX

SEQ ID NO: 308 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 309 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 8: note = C-term NH2
RSDTLXWE

SEQ ID NO: 310 - protein, 9 aa, synthetic construct
  REGION 1..9: note = Description of Artificial Sequence: Synthetic peptide
  REGION 1..6: note = CROSSLINK - N-term 3-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 311 - protein, 9 aa, synthetic construct
  REGION 1..9: note = Description of Artificial Sequence: Synthetic peptide
  REGION 1..6: note = CROSSLINK - N-term 4-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 312 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXWEK

SEQ ID NO: 313 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 8: note = D-Lys
  SITE 8: note = C-term OH
RSDTLCXK

SEQ ID NO: 314 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 8: note = L-glutamic acid g-methyl ester
  SITE 9: note = D-Lys
  SITE 9: note = C-term NH2
RSDTLXWEK

SEQ ID NO: 315 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = D-Phe
  SITE 9: note = D-Lys
  SITE 9: note = C-term NH2
RSDTLXXFK

SEQ ID NO: 316 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = D-His
  SITE 9: note = D-Lys
  SITE 9: note = C-term NH2
RSDTLXXHK

SEQ ID NO: 317 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = D-Leu
  SITE 9: note = D-Lys
  SITE 9: note = C-term NH2
RSDTLXXLK

SEQ ID NO: 318 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = D-Arg
  SITE 9: note = D-Lys
  SITE 9: note = C-term NH2
RSDTLXXRK

SEQ ID NO: 319 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = (3S-)-1,2,3,4-tetrahydroisoquinoline-3-carboxylic acid
  SITE 9: note = D-Lys
  SITE 9: note = C-term NH2
RSDTLXXXK

SEQ ID NO: 320 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = D-Thr
  SITE 9: note = D-Lys
  SITE 9: note = C-term NH2
RSDTLXXTK

SEQ ID NO: 321 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 9: note = D-Lys
  SITE 9: note = C-term NH2
RSDTLXXEK

SEQ ID NO: 322 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = Beta-Homo-Glutamic acid
  SITE 9: note = D-Lys
  SITE 9: note = C-term NH2
RSDTLXXXK

SEQ ID NO: 323 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  MOD_RES 9: note = N-Methyl Lysine
  SITE 9: note = C-term NH2
RSDTLXXEK

SEQ ID NO: 324 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  MOD_RES 9: note = D-Form N-Methyl Lysine
  SITE 9: note = C-term NH2
RSDTLXXEK

SEQ ID NO: 325 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = Beta-Homo-Glutamic acid
  MOD_RES 9: note = N-Methyl Lysine
  SITE 9: note = C-term NH2
RSDTLXXXK

SEQ ID NO: 326 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 1-Napthylalanine
  SITE 8: note = Beta-Homo-Glutamic acid
  MOD_RES 9: note = D-Form N-Methyl Lysine
  SITE 9: note = C-term NH2
RSDTLXXXK

SEQ ID NO: 327 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Phe
  SITE 9: note = D-Lys
  SITE 9: note = C-term NH2
RSDTLXXFK

SEQ ID NO: 328 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-His
  SITE 9: note = D-Lys
  SITE 9: note = C-term NH2
RSDTLXXHK

SEQ ID NO: 329 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Leu
  SITE 9: note = D-Lys
  SITE 9: note = C-term NH2
RSDTLXXLK

SEQ ID NO: 330 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Arg
  SITE 9: note = D-Lys
  SITE 9: note = C-term NH2
RSDTLXXRK

SEQ ID NO: 331 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = (3S-)-1,2,3,4-tetrahydroisoquinoline-3-carboxylic acid
  SITE 9: note = D-Lys
  SITE 9: note = C-term NH2
RSDTLXXXK

SEQ ID NO: 332 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 9: note = D-Lys
  SITE 9: note = C-term NH2
RSDTLXXEK

SEQ ID NO: 333 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = Beta-Homo-Glutamic acid
  SITE 9: note = D-Lys
  SITE 9: note = C-term NH2
RSDTLXXXK

SEQ ID NO: 334 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Lys
  SITE 8: note = C-term NH2
RSDTLXXK

SEQ ID NO: 335 - protein, 8 aa, synthetic construct
  REGION 1..8: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = D-Lys
  SITE 8: note = C-term NH2
RSDTLXXK

SEQ ID NO: 336 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  MOD_RES 9: note = N-Methyl Lysine
  SITE 9: note = C-term NH2
RSDTLXXEK

SEQ ID NO: 337 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  MOD_RES 9: note = D-Form N-Methyl Lysine
  SITE 9: note = C-term NH2
RSDTLXXEK

SEQ ID NO: 338 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = Beta-Homo-Glutamic acid
  MOD_RES 9: note = N-Methyl Lysine
  SITE 9: note = C-term NH2
RSDTLXXXK

SEQ ID NO: 339 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 2-Napthylalanine
  SITE 8: note = Beta-Homo-Glutamic acid
  MOD_RES 9: note = D-Form N-Methyl Lysine
  SITE 9: note = C-term NH2
RSDTLXXXK

SEQ ID NO: 340 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 8: note = D-Glu
  SITE 9: note = D-Lys
  SITE 9: note = C-term NH2
RSDTLXXEK

SEQ ID NO: 341 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 9: note = D-Lys
  SITE 9: note = C-term NH2
RSDTLXXEK

SEQ ID NO: 342 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 8: note = Beta-Homo-Glutamic acid
  SITE 9: note = D-Lys
  SITE 9: note = C-term NH2
RSDTLXXXK

SEQ ID NO: 343 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  MOD_RES 9: note = N-Methyl Lysine
  SITE 9: note = C-term NH2
RSDTLXXEK

SEQ ID NO: 344 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 9: note = NOD_RES - D-Form N-Methyl Lysine
  SITE 9: note = C-term NH2
RSDTLXXEK

SEQ ID NO: 345 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 8: note = Beta-Homo-Glutamic acid
  MOD_RES 9: note = N-Methyl Lysine
  SITE 9: note = C-term NH2
RSDTLXXXK

SEQ ID NO: 346 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = Biphenylalanine
  SITE 8: note = Beta-Homo-Glutamic acid
  MOD_RES 9: note = D-Form N-Methyl Lysine
  SITE 9: note = C-term NH2
RSDTLXXXK

SEQ ID NO: 347 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S)- 2-amino-3-(2,4-dichlorophenyl)propionic acid
  SITE 8: note = D-Glu
  SITE 9: note = D-Lys
  SITE 9: note = C-term NH2
RSDTLXXEK

SEQ ID NO: 348 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = L-2-carbamoylphenylalanine
  SITE 8: note = D-Glu
  SITE 9: note = D-Lys
  SITE 9: note = C-term NH2
RSDTLXXEK

SEQ ID NO: 349 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S)- 2-amino-3-(3,4-dichlorophenyl)propionic acid
  SITE 8: note = D-Glu
  SITE 9: note = D-Lys
  SITE 9: note = C-term NH2
RSDTLXXEK

SEQ ID NO: 350 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = L-3-carbamoylphenylalanine
  SITE 8: note = D-Glu
  SITE 9: note = D-Lys
  SITE 9: note = C-term NH2
RSDTLXXEK

SEQ ID NO: 351 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = 4-Trifluoro-Phenylalanine
  SITE 8: note = D-Glu
  SITE 9: note = D-Lys
  SITE 9: note = C-term NH2
RSDTLXXEK

SEQ ID NO: 352 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 8: note = D-Glu
  SITE 9: note = D-Lys
  SITE 9: note = C-term NH2
RSDTLXFEK

SEQ ID NO: 353 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 9: note = D-Lys
  SITE 9: note = C-term NH2
RSDTLXFEK

SEQ ID NO: 354 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 9: note = D-Lys
  SITE 9: note = C-term NH2
RSDTLXFEK

SEQ ID NO: 355 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 9: note = D-Lys
  SITE 9: note = C-term NH2
RSDTLXFEK

SEQ ID NO: 356 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 8: note = Beta-Homo-Glutamic acid
  SITE 9: note = D-Lys
  SITE 9: note = C-term NH2
RSDTLXFXK

SEQ ID NO: 357 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 8: note = Beta-Homo-Glutamic acid
  SITE 9: note = D-Lys
  SITE 9: note = C-term NH2
RSDTLXFXK

SEQ ID NO: 358 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-COOH)
  SITE 8: note = Beta-Homo-Glutamic acid
  MOD_RES 9: note = D-Lysine
  SITE 9: note = C-term NH2
RSDTLXFXK

SEQ ID NO: 359 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = 4-fluoro-phenylalanine
  SITE 8: note = D-Glu
  SITE 9: note = D-Lys
  SITE 9: note = C-term NH2
RSDTLXFEK

SEQ ID NO: 360 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 7: note = Phe(4-OMe)
  SITE 8: note = D-Glu
  SITE 9: note = D-Lys
  SITE 9: note = C-term NH2
RSDTLXFEK

SEQ ID NO: 361 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = D-Glu
  SITE 9: note = D-Lys
  SITE 9: note = C-term NH2
RSDTLXXEK

SEQ ID NO: 362 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  MOD_RES 9: note = N-Methyl Lysine
  SITE 9: note = C-term NH2
RSDTLXXEK

SEQ ID NO: 363 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = Beta-Homo-Glutamic acid
  SITE 9: note = D-Lys
  SITE 9: note = C-term NH2
RSDTLXXXK

SEQ ID NO: 364 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  MOD_RES 9: note = N-Methyl Lysine
  SITE 9: note = C-term NH2
RSDTLXXEK

SEQ ID NO: 365 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  MOD_RES 9: note = D-Form N-Methyl Lysine
  SITE 9: note = C-term NH2
RSDTLXXEK

SEQ ID NO: 366 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = Beta-Homo-Glutamic acid
  MOD_RES 9: note = N-Methyl Lysine
  SITE 9: note = C-term NH2
RSDTLXXXK

SEQ ID NO: 367 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 7: note = (S) -2-amino-3-(4-tert-butyl-phenyl)propionic acid
  SITE 8: note = Beta-Homo-Glutamic acid
  MOD_RES 9: note = D-Form N-Methyl Lysine
  SITE 9: note = C-term NH2
RSDTLXXXK

SEQ ID NO: 368 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXHEK

SEQ ID NO: 369 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 8: note = D-Glu
  SITE 9: note = D-Lys
  SITE 9: note = C-term NH2
RSDTLCXEK

SEQ ID NO: 370 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 7: note = (3S-)-1,2,3,4-tetrahydroisoquinoline-3-carboxylic acid
  SITE 8: note = D-Glu
  SITE 9: note = D-Lys
  SITE 9: note = C-term NH2
RSDTLCXEK

SEQ ID NO: 371 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = Diaminobutyric acid
  SITE 9: note = C-term NH2
RSDTLXWEX

SEQ ID NO: 372 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Phe
  SITE 9: note = D-Lys
  SITE 9: note = C-term NH2
RSDTLXWFK

SEQ ID NO: 373 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Tyr
  SITE 9: note = D-Lys
  SITE 9: note = C-term NH2
RSDTLXWYK

SEQ ID NO: 374 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = D-Lys
  SITE 9: note = C-term NH2
RSDTLXWPK

SEQ ID NO: 375 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 9: note = C-term NH2
RSDTLXWPK

SEQ ID NO: 376 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Pro
  SITE 9: note = C-term NH2
RSDTLXWPK

SEQ ID NO: 377 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-His
  SITE 9: note = D-Lys
  SITE 9: note = C-term NH2
RSDTLXWHK

SEQ ID NO: 378 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  MOD_RES 8: note = Phe(4-COOH)
  SITE 9: note = D-Lys
  SITE 9: note = C-term NH2
RSDTLXWFK

SEQ ID NO: 379 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = (3S-)-1,2,3,4-tetrahydroisoquinoline-3-carboxylic acid
  SITE 9: note = D-Lys
  SITE 9: note = C-term NH2
RSDTLXWXK

SEQ ID NO: 380 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Trp
  SITE 9: note = D-Lys
  SITE 9: note = C-term NH2
RSDTLXWWK

SEQ ID NO: 381 - protein, 8 aa, synthetic construct
  REGION 1..8: note = Description of Artificial Sequence: Synthetic peptide
  REGION 1..6: note = CROSSLINK - N-term Acetyl with a thioether bond between the N-term and the residue at position 6
  MOD_RES 1: note = N-Methyl Arginine
  SITE 6: note = Penicillamine
  SITE 8: note = D-Lys
  SITE 8: note = C-term NH2
RSDTLXWK

SEQ ID NO: 382 - protein, 8 aa, synthetic construct
  REGION 1..8: note = Description of Artificial Sequence: Synthetic peptide
  REGION 1..6: note = CROSSLINK - N-term Propionyl with a thioether bond between the N-term and the residue at position 6
  MOD_RES 1: note = N-Methyl Arginine
  SITE 6: note = Penicillamine
  SITE 8: note = D-Lys
  SITE 8: note = C-term NH2
RSDTLXWK

SEQ ID NO: 383 - protein, 8 aa, synthetic construct
  REGION 1..8: note = Description of Artificial Sequence: Synthetic peptide
  REGION 1..6: note = CROSSLINK - N-term Propionyl with a thioether bond between the N-term and the residue at position 6
  MOD_RES 1: note = N-Methyl Arginine
  SITE 6: note = homo-Cysteine
  SITE 8: note = D-Lys
  SITE 8: note = C-term NH2
RSDTLXWK

SEQ ID NO: 384 - protein, 9 aa, synthetic construct
  REGION 1..9: note = Description of Artificial Sequence: Synthetic peptide
  REGION 1..6: note = CROSSLINK - N-term 2-Benzyl with a thioether bond between the N-term and the residue at position 6
  MOD_RES 1: note = N-Methyl Arginine
  SITE 6: note = Penicillamine
  SITE 8: note = D-Glu
  SITE 9: note = D-Lys
  SITE 9: note = C-term NH2
RSDTLXYEK

SEQ ID NO: 385 - protein, 9 aa, synthetic construct
  REGION 1..9: note = Description of Artificial Sequence: Synthetic peptide
  SITE 1: note = N-term Ac
  SITE 9: note = C-term NH2
CRSDTLCGE

SEQ ID NO: 386 - protein, 7 aa, synthetic construct
  REGION 1..7: note = Description of Artificial Sequence: Synthetic peptide
  SITE 1: note = N-term Ac
  SITE 7: note = C-term NH2
CRSDTLC

SEQ ID NO: 387-389 - skipped (3 entries)

SEQ ID NO: 390 - protein, 6 aa, synthetic construct
  REGION 1..6: note = Description of Artificial Sequence: Synthetic peptide
  REGION 1..6: note = CROSSLINK - N-term 2-methyl benzoyl with a thioether bond between the N-term and the residue at position 6
  MOD_RES 1: note = N-Methyl Arginine
  VARIANT 6: note = Penicillamine, Cys, D-Cys or HomoCys
RSETLX

SEQ ID NO: 391 - protein, 8 aa, synthetic construct
  REGION 1..8: note = Description of Artificial Sequence: Synthetic peptide
  REGION 1..6: note = CROSSLINK - N-term Acetyl with a thioether bond between the N-term and the residue at position 6
  MOD_RES 1: note = N-Methyl Arginine
  SITE 8: note = D-Lys
  SITE 8: note = C-term NH2
RSDTLCWK

SEQ ID NO: 392 - protein, 8 aa, synthetic construct
  REGION 1..8: note = Description of Artificial Sequence: Synthetic peptide
  REGION 1..6: note = CROSSLINK - N-term Acetyl with a thioether bond between the N-term and the residue at position 6
  MOD_RES 1: note = N-Methyl Arginine
  SITE 6: note = homo-Cysteine
  SITE 8: note = D-Lys
  SITE 8: note = C-term NH2
RSDTLXWK

SEQ ID NO: 393 - skipped

SEQ ID NO: 394 - protein, 8 aa, synthetic construct
  REGION 1..8: note = Description of Artificial Sequence: Synthetic peptide
  REGION 1..6: note = CROSSLINK - N-term Aromatic acid group, 2-methylbenzoyl, propionyl, isobutyryl, acetyl or butyryl forming a thioether bond with Xaa at position 6
  MOD_RES 1: note = Optional N(alpha)methylation
  VARIANT 6: note = Cys, Pen, HCys, D-Cys
  VARIANT 8: note = Glu, D-Glu, D-Lysine
RSDTLXWX